(19) United States
(12) Patent Application Publication
(10) Pub. No.: US 2023/0364255 A1
(43) Pub. Date: Nov. 16, 2023

(54) TRANSGLUTAMINASE-MEDIATED CONJUGATION

(71) Applicant: Tallac Therapeutics, Inc., Burlingame, CA (US)

(72) Inventors: Curt W. BRADSHAW, San Francisco, CA (US); Son LAM, San Francisco, CA (US); Edward HyungSuk HA, Cambridge, MA (US); Sukumar SAKAMURI, San Diego, CA (US)

(21) Appl. No.: 18/021,372

(22) PCT Filed: Aug. 17, 2021
(86) PCT No.: PCT/US2021/046287

§ 371 Date: Feb. 14, 2023

Related U.S. Application Data
(60) Provisional application No. 63/067,113, filed on Aug. 18, 2020.

(51) Int. Cl. A61K 47/68

(52) U.S. Cl. CPC A61K 47/6807; A61K 47/6835

(57) ABSTRACT

The present disclosure provides methods for conjugating an oligonucleotide and a polypeptide via a transglutaminase-mediated reaction. The conjugates of the present disclosure comprise a linker moiety that provides better stability of the conjugate. Also provided are related compounds, compositions and kits.

### CROSS REFERENCE TO RELATED APPLICATION

This application claims priority to and the benefit of U.S. Provisional Pat. Application No. 63/067,113, filed Aug. 18, 2020, the disclosure of which is herein incorporated by reference in its entirety.

### FIELD OF INVENTION

The present disclosure relates generally to methods for conjugating an oligonucleotide and a polypeptide and related compounds, compositions and kits.

### BACKGROUND

Pathogen-associated molecular patterns (PAMPs) are molecules associated with various pathogens and are recognized by toll-like receptors (TLRs) and other pattern recognition receptors (PRRs) activating innate immune responses. The ability of PAMPs to recruit immune system in the absence of pathogens provides a strategy for treating a variety of diseases involving cell destruction (e.g., anticancer therapy) through the use of innate immune system response. One class of PAMPs that has been investigated for a variety of therapeutic applications is immunostimulating polynucleotides, such as unmethylated cytosine-guanine dinucleotide (CpG)-containing oligodeoxynucleotides (CpG ODNs) (e.g., agatolimod). It is thought that CpG ODNs mediate TLR9 dimerization in immune cells (e.g., B cells, monocytes and plasmacytoid dendritic cells (pDCs)) to upregulate cytokines (e.g., type I interferon and interleukins), thereby activating natural killer cells.

Toll-like receptor 9 (TLR9), also designated as CD289, is an important receptor expressed in immune system cells including dendritic cells (DCs), B lymphocytes, macrophages, natural killer cells, and other antigen presenting cells. TLR9 activation triggers intracellular signaling cascades, leading to activation, maturation, proliferation and cytokine productions in these immune cells, thus bridges the innate and adaptive immunity. Martinez-Campos et al., Viral Immunol. 2016, 30, 98-105; Notley et al., Sci. Rep. 2017, 7, 42204. Natural TLR-9 agonists include unmethylated cytosine-guanine dinucleotide (CpG)-containing oligodeoxynucleotides (CpG ODNs).

CpG ODNs may include, for example, oligodeoxynucleotides having poly-G tails with phosphorothioate backbones at 3′- and 5′-termini and a central palindromic sequence including a phosphate backbone and a CpG within its central palindrome sequence, or oligodeoxynucleotides having a fully phosphorothioate backbone, and a sequence at the 5′ end for TLR9 activation, or oligodeoxynucleotides having a fully phosphorothioate backbone with a 3′-end sequence enabling formation of a duplex. However, CpG ODNs are often susceptible to degradation in serum and thus pharmacokinetics of CpG ODNs may be one of the limiting factors in their development as therapeutics. Also CpG ODNs often exhibit uneven tissue distribution in vivo, with primary sites of accumulation being in liver, kidney, and spleen. Such distribution can elicit off-target activity and local toxicity associated with PAMPs.

One solution is to conjugate the immunomodulating polynucleotides (e.g., CpG ODNs) with a targeting moiety for specifically targeted tissues or cells to overcome the uneven distribution of the polynucleotide. See US 2018/0312536. Particularly, transglutaminase-mediated reaction can be used to conjugate a polypeptide targeting moiety containing a glutamine residue with a CpG ODN containing a primary amine group. Microbial transglutaminase (mTG) is from the species Streptomyces mobaraensis. The mTG catalyzes under pH-controlled aqueous conditions (including physiological conditions) a transamidation reaction between a ‘reactive’ glutamine of a protein and a ‘reactive’ lysine residue whereas the latter can also be a simple, low molecular weight primary amine such as a 5-aminopentyl group. For an endogenous glutamine on a protein to be recognized as an mTG-substrate two criteria seem important: 1) the presence of hydrophobic amino acids in the peptide sequence adjacent to the glutamine; and 2) the positioning of the glutamine on a loop with local chain flexibility enhancing reactivity toward mTG.

However, drawbacks still exist. For example, transglutaminase (Tgase) deamidation leads to the formation of glutamic acid which does not conjugate. To achieve a high degree of conjugation, the ratio of amine to glutamine residue typically needs to be >50:1. Deconjugation occurs even at low temperatures (e.g., 4° C.). Benchmark Q-tag (LLQGG) deconjugates and can be proteolyzed by Tgase giving LLEGG and LLE. Removal of Tgase is inefficient using commercially available methods (e.g., size exclusion chromatography).

Although conjugation of these immunomodulating polynucleotides may lead to improved stability and distribution, there remains a need for immunomodulating polynucleotides with improved stability and selectivity and methods for preparing them.

### BRIEF SUMMARY

Provided herein is a conjugate of Formula (A1):

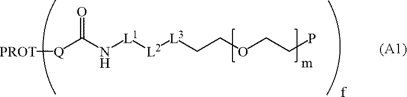

or a stereoisomer, a mixture of two or more diastereomers, a tautomer, or a mixture of two or more tautomers thereof; or a pharmaceutically acceptable salt, solvate, or hydrate thereof; wherein:
Q is a glutamine residue, wherein each glutamine residue independently is part of the sequence of PROT or is part of a Q-tag peptide sequence attached to PROT;
PROT is a protein connected to the rest of the conjugate via one or more glutamine residues Q;
each L¹ is independently unsubstituted or substituted alkyl,
each L² is unsubstituted or substituted aryl, or unsubstituted or substituted heteroaryl,
each L³ is independently absent or a linker moiety,
f is an integer selected from the group consisting of 1-20,
m is an integer selected from the group consisting of 0-50, and
P is an immunomodulating oligonucleotide.

In some embodiments, P is

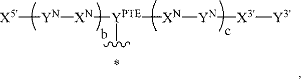

wherein
b and c are each independently an integer from 1 to 25; with the proviso that the sum of b and c is at least 5;
〰 * indicates the point of attachment of the immunomodulating oligonucleotide P to the rest of the conjugate;
X⁵’ is a 5′ terminal nucleoside having the structure

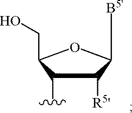

X³’ is a 3′ terminal nucleoside having the structure

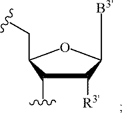

Y^{PTE} is an internucleoside phosphotriester having the structure

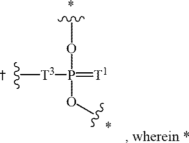

indicates the points of attachment to the rest of the oligonucleotide and indicates the point of attachment to the rest of the conjugate;
Y^{3′} is a terminal phosphotriester having the structure

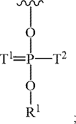

each X^{N} is independently a nucleoside having the structure

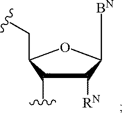

each Y^{N} is independently an internucleoside linker having the structure

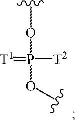

wherein each B^{N} is independently a modified or unmodified nucleobase;
each R^{N} is independently —H or —O—C₁₋₄-alkyl, wherein the C₁₋₄-alkyl of the —O—C₁₋₄-alkyl is further optionally substituted by —O—C₁-C₄-alkyl;
B^{5′} and B³’ are independently a modified or unmodified nucleobase;
R⁵’ and R³’ are independently —H or —O—C₁-C₄-alkyl, wherein the C₁₋₄-alkyl of the —O—C₁₋₄alkyl is further optionally substituted by —O—C₁-C₄-alkyl;
each T₁ is independently O or S;
each T₂ is independently O⁻ or S⁻; and
T₃ is a group comprising an oligoethylene glycol moiety; and
R¹ is C₁₋₄-alkylene-hydroxy.

In some embodiments, (i) P comprises at least one modified nucleoside X^{N}; (ii) P has at least one modified internucleoside linker Y^{N}, wherein at least one of T¹ or T² is S; or (iii) both (i) and (ii). In some embodiments, at least one Y^{N} is a phosphorodithioate or phosphorothioate linker. In certain embodiments, P comprises 0, 1, 2 or 3 phosphorodithioate linkers. In some embodiments, P comprises one or more CpG sites. In some embodiments, the modified nucleoside is selected from the group consisting of 2′-O-alkyl nucleotide, 2′-O-alkoxyalkyl nucleoside, 2′-deoxy nucleoside and ribonucleoside. In some embodiments, the modified nucleoside is selected from the group consisting of 5-bromo-2′-O-methyluridine, 5-bromo-2′-O-methyl-deoxyuridine, 5-bromo-2′-deoxyuridine, 2′-O-methylthymidine, 2′-O-methylcytidine, 2′-O-(2-methoxyethyl)thymidine and 8-oxo-7,8-dihydro-2′-deoxyguanosine. In yet other embodiments, Y^{3′} or the Y^{N} at the 3′ position of X^{5′} comprises an unsubstituted or substituted phosphorothioate. In some embodiments, P comprises an oligonucleotide sequence selected from the group consisting of SEQ ID NOS: 1-5. In some embodiments, m is 15-30. In some embodiments, Y^{PTE} is:

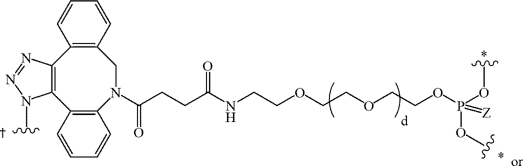

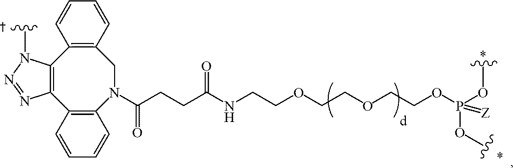

wherein Z is O or S; d is an integer from 0 to 95; the two 〰 * on the right side of the structure indicate the points of attachment to the adjacent nucleosides X^{N} in the oligonucleotide P, and the one on the left side of the structure indicates the point of attachment to the rest of the conjugate. In some embodiments, Y^{PTE} is:

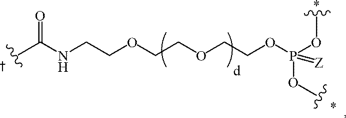

wherein Z is O or S; d is an integer from 0 to 95; the two 〰 * on the right side of the structure indicate the points of attachment to the adjacent nucleosides X^{N} in the oligonucleotide P, and the on the left side of the structure indicates the point of attachment to the rest of the conjugate. In some embodiments, Z is S.

In some embodiments, each L¹ is independently methyl or ethyl. In some embodiments, each L¹ is independently substituted by an unsubstituted or substituted aryl. In some embodiments, each L¹ is substituted by phenyl. In other embodiments, each L² is independently a 6-10 membered aryl. In some embodiments, each L² is phenyl. In some embodiments, each L² is independently a 6-10 membered heteroaryl. In some embodiments, each L² is pyridinyl. In some embodiments, each L³ is a linker moiety. In some embodiments, the linker moiety is an unsubstituted or substituted alkyl. In some embodiments, the linker is methyl. In some embodiments, the linker moiety is —R¹C(O)R²NHR³—, wherein R¹ and R³ are independently absent or unsubstituted or substituted alkyl and R² is an amino acid residue. In some embodiments, the amino acid is selected from the group consisting of glycine, alanine, glutamic acid and proline. In some embodiments, the linker moiety is —R⁴C(O)NHR⁵— or —R⁴NHC(O)R⁵—, wherein R⁴ and R⁵ are independently absent or unsubstituted or substituted alkyl. In some embodiments, R⁴ is methylene and R⁵ is —(CH₂)₄—. In some embodiments, R⁴ is methylene and R⁵ is absent.

In some embodiments, the Q-tag comprises a sequence selected from the group consisting of SEQ ID NOS: 6-18. In some embodiments, the protein is an antibody. In some embodiments, the protein is an antibody, e.g., a monoclonal antibody. In some embodiments, the antibody is an antibody fragment selected from the group consisting of Fab, F(ab′)2, Fab′-SH, Fv, scFv, single domain, single heavy chain, and single light chain antibody fragments. In some embodiments, the antibody is a human, humanized, or chimeric antibody. In some embodiments, the antibody comprises an Fc region. In some embodiments, the antibody comprises a human Fc region, e.g., a human IgG1, human IgG2, human IgG3, or human IgG4 antibody. In some embodiments, the antibody binds to human CD22. In some embodiments, the antibody is an anti-CD22 antibody. In some embodiments, the antibody is a human or humanized anti-CD22 antibody. In some embodiments, the antibody comprises a light chain variable domain (VL) and a heavy chain variable domain (VH). In some embodiments, the glutamine residue Q is within the C-terminus of the heavy chain of the antibody, or the Q-tag peptide sequence comprising the glutamine residue Q is attached to the C-terminus of the heavy chain of the antibody. In some embodiments, the glutamine residue Q is within the light chain of the antibody or the Q-tag peptide sequence comprising the glutamine residue Q is attached to the light chain of the antibody. In some embodiments, the glutamine residue or the Q-tag peptide sequence comprising the glutamine residue Q is naturally occurring. For example, mutation of N297 to N297A exposes Q295 of the antibody, where the conjugation could occur. In some embodiments, the glutamine residue Q or the Q-tag peptide sequence comprising the glutamine residue Q is within the Fc region of the antibody. In some embodiments, which may be combined with any of the preceding embodiments, f is 1 or 2. In some embodiments, m is 23.

In some embodiments, the conjugate of Formula (A1) is a conjugate of Formula (A2):

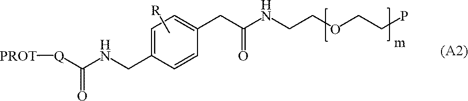

wherein R is one or more substituents selected from the group consisting of H, halo, hydroxyl, substituted or unsubstituted alkyl, substituted or unsubstituted alkenyl, substituted or unsubstituted alkynyl, substituted or unsubstituted hydroxyalkyl, substituted or unsubstituted cycloalkyl, substituted or unsubstituted cycloalkenyl, substituted or unsubstituted heteroaryl, substituted or unsubstituted alkoxy, or substituted or unsubstituted aryl.

Also provided herein is a conjugate of Formula (B1):

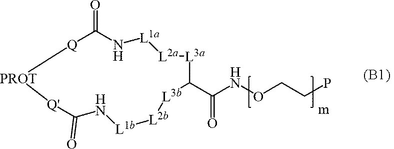

or a stereoisomer, a mixture of two or more diastereomers, a tautomer, or a mixture of two or more tautomers thereof; or a pharmaceutically acceptable salt, solvate, or hydrate thereof; wherein:
Q and Q′ are each a glutamine residue, wherein each glutamine residue independently is part of the sequence of PROT or is part of a Q-tag peptide sequence attached to PROT;
PROT is a protein connected to the rest of the conjugate via Q and Q′;
L^{1a} and L^{1b} are independently unsubstituted or substituted alkyl,
L^{2a} and L^{2b} are independently absent, unsubstituted or substituted alkyl, unsubstituted or substituted aryl, or unsubstituted or substituted heteroaryl,
L^{3a} and L^{3b} are independently absent or a linker moiety,
m is an integer selected from the group consisting of 0-50, and
P is an immunomodulating oligonucleotide.

In some embodiments, P is

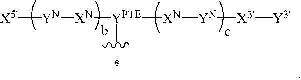

wherein
b and c are each independently an integer from 1 to 25; with the proviso that the sum of b and c is at least 5;
〰 * indicates the point of attachment of the immunomodulating oligonucleotide P to the rest of the conjugate;
X^{5′} is a 5′ terminal nucleoside having the structure

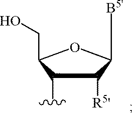

X^{3′} is a 3′ terminal nucleoside having the structure

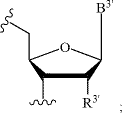

Y^{PTE} is an internucleoside phosphotriester having the structure

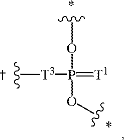

wherein * indicates the points of attachment to the rest of the oligonucleotide and indicates the point of attachment to the rest of the conjugate;
Y^{3′} is a terminal phosphotriester having the structure

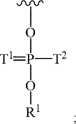

each X^{N} is independently a nucleoside having the structure

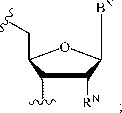

each Y^{N} is independently an internucleoside linker having the structure

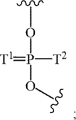

wherein each B^{N} is independently a modified or unmodified nucleobase;
each R^{N} is independently —H or —O—C₁₋₄-alkyl, wherein the C₁₋₄-alkyl of the —O—C₁₋₄-alkyl is further optionally substituted by —O—C₁-C₄-alkyl;
B^{5′} and B^{3′} are independently a modified or unmodified nucleobase;
R^{5′} and R^{3′} are independently —H or —O—C₁-C₄-alkyl, wherein the C₁₋₄-alkyl of the —O—C₁₋₄alkyl is further optionally substituted by —O—C₁-C₄-alkyl;
each T₁ is independently O or S;
each T₂ is independently O⁻ or S⁻; and
T₃ is a group comprising an oligoethylene glycol moiety; and
R¹ is C₁₋₄-alkylene-hydroxy.

In some embodiments, (i) P comprises at least one modified nucleoside X^{N}; (ii) P has at least one modified internucleoside linker Y^{N}, wherein at least one of T¹ or T² is S; or (iii) both (i) and (ii). In some embodiments, at least one Y^{N} is a phosphorodithioate or phosphorothioate linker. In certain embodiments, P comprises 0, 1, 2 or 3 phosphorodithioate linkers. In some embodiments, P comprises one or more CpG sites. In some embodiments, the modified nucleoside is selected from the group consisting of 2′-O-alkyl nucleoside, 2′-O-alkoxyalkyl nucleoside, 2′-deoxy nucleoside and ribonucleoside. In some embodiments, the modified nucleoside is selected from the group consisting of 5-bromo-2′-O-methyluridine, 5-bromo-2′-O-methyl-deoxyuridine, 5-bromo-2′-deoxyuridine, 2′-O-methylthymidine, 2′-O-methylcytidine, 2′-O-(2-methoxyethyl)thymidine and 8-oxo-7,8-dihydro-2′-deoxyguanosine. In yet other embodiments, Y^{3′} or the Y^{N} at the 3′ position of X^{5′} comprises an unsubstituted or substituted phosphorothioate. In some embodiments, P comprises an oligonucleotide sequence selected from the group consisting of SEQ ID NOS: 1-5. In some embodiments, m is 15-30. In some embodiments, Y^{PTE} is:

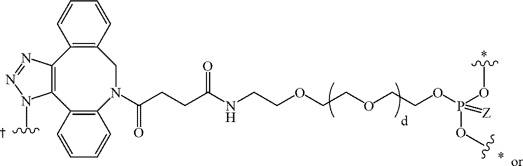

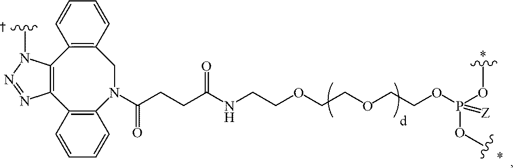

wherein Z is O or S; d is an integer from 0 to 95; the two 〰 * on the right side of the structure indicate the points of attachment to the adjacent nucleosides X^{N} in the oligonucleotide P, and the one on the left side of the structure indicates the point of attachment to the rest of the conjugate. In some embodiments, Y^{PTE} is:

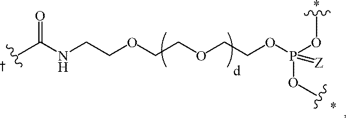

wherein Z is O or S; d is an integer from 0 to 95; the two 〰 * on the right side of the structure indicate the points of attachment to the adjacent nucleosides X^{N} in the oligonucleotide P, and the on the left side of the structure indicates the point of attachment to the rest of the conjugate. In some embodiments, Z is S.

In some embodiments, one or both of L^{1a} and L^{1b} are methyl or ethyl. In some embodiments, one or both of L^{1a} and L^{1b} are substituted by an unsubstituted or substituted aryl. In some embodiments, one or both of L^{1a} and L^{1b} are absent. In some embodiments, one or both of L^{3a} and L^{3b} are linker moieties. In some embodiments, the linker moiety is an unsubstituted or substituted alkyl. In some embodiments, the linker is methyl. In some embodiments, the linker moiety is —R¹C(O)R²NHR³—, wherein R¹ and R³ are independently absent or unsubstituted or substituted alkyl and R² is an amino acid residue. In some embodiments, the amino acid is selected from the group consisting of glycine, alanine, glutamic acid and proline. In some embodiments, the linker moiety is —R⁴C(O)NHR⁵— or —R⁴NHC(O)R⁵—, wherein R⁴ and R⁵ are independently absent or unsubstituted or substituted alkyl. In some embodiments, R⁴ is methylene and R⁵ is —(CH₂)₄—. In some embodiments, R⁴ is methylene and R⁵ is absent.

In some embodiments, the Q-tag peptide sequences comprising Q and Q′ are the same. In some embodiments, the Q-tag peptide sequences comprising Q and Q′ independently comprise a peptide sequence selected from the group consisting of SEQ ID NOs: 6-18. In some embodiments, the protein is an antibody. In some embodiments, the protein is an antibody, e.g., a monoclonal antibody. In some embodiments, the antibody is an antibody fragment selected from the group consisting of Fab, F(ab′)2, Fab′-SH, Fv, scFv, single domain, single heavy chain, and single light chain antibody fragments. In some embodiments, the antibody is a human, humanized, or chimeric antibody. In some embodiments, the antibody comprises an Fc region. In some embodiments, the antibody comprises a human Fc region, e.g., a human IgG1, human IgG2, human IgG3, or human IgG4 antibody. In some embodiments, the antibody binds to human CD22. In some embodiments, the antibody is an anti-CD22 antibody. In some embodiments, the antibody is a human or humanized anti-CD22 antibody. In some embodiments, the antibody comprises a light chain variable domain (VL) and a heavy chain variable domain (VH). In some embodiments, the glutamine residues Q and Q′ or the Q-tag peptide sequences comprising Q and Q′ are attached to the antibody at different points.

In some embodiments, the glutamine residue Q or Q′ is independently within the C terminus of the heavy chain of the antibody or within the Fc domain of the antibody. In some embodiments, the glutamine residue Q or Q′ is independently within the light chain of the antibody. In some embodiments, the glutamine residue Q or Q′ is naturally occurring. In some embodiments, the Q-tag peptide sequence comprising Q or Q′ is independently attached to the C terminus of the heavy chain of the antibody or within the Fc domain of the antibody. In some embodiments, the Q-tag peptide sequence comprising Q or Q′ is attached to the light chain of the antibody. In some embodiments, the Q-tag peptide sequence comprising Q or Q′ is naturally occurring. For example, mutation of N297 to N297A exposes Q295 of the antibody, where the conjugation could occur. In some embodiments, m is 23.

Also provided herein is a method of preparing of a conjugate of Formula (A1):

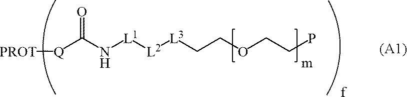

or a stereoisomer, a mixture of two or more diastereomers, a tautomer, or a mixture of two or more tautomers thereof; or a pharmaceutically acceptable salt, solvate, or hydrate thereof; comprising reacting (1) a compound of Formula (I):

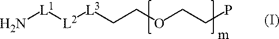

or a stereoisomer, a mixture of two or more diastereomers, a tautomer, or a mixture of two or more tautomers thereof; or a pharmaceutically acceptable salt, solvate, or hydrate thereof, and (2) a protein comprising one or more glutamine residues in the presence of a transglutaminase, wherein:
Q is a glutamine residue, wherein each glutamine residue independently is part of the sequence of PROT or is part of a Q-tag peptide sequence attached to PROT;
PROT is a protein connected to the rest of the conjugate via one or more glutamine residues Q;
each L¹ is independently unsubstituted or substituted alkyl,
each L² is unsubstituted or substituted aryl, or unsubstituted or substituted heteroaryl,
each L³ is independently absent or a linker moiety,
f is an integer selected from the group consisting of 1-20,
m is an integer selected from the group consisting of 0-50, and
P is an immunomodulating oligonucleotide.

In some embodiments, the ratio of compound (I) and the protein is in the range of 1:1 to 1:10 by molarity. In certain embodiments wherein the protein is an antibody, the ratio of compound (I) and the antibody is in the range of 1:1 to 1:10 by molarity.

In some embodiments, the molar ratio of the protein-to-transglutaminase is in the range of 1:5 to 1:20. In certain embodiments, the molar ratio of the protein-to-transglutaminase is in the range of 1:10 to 1:18. In other embodiments, the molar ratio of the protein-to-transglutaminase is at least 1:5, at least 1:6, at least 1:7, at least 1:8, at least 1:9, at least 1:10, at least 1:11, at least 1:12, at least 1:13, at least 1:14, at least 1:15, at least 1:16, or at least 1:17. In yet other embodiments, the molar ratio of the protein-to-transglutaminase is less than or equal to 1:20, less than or equal to 1:19, less than or equal to 1:18, less than or equal to 1:17, less than or equal to 1:17, less than or equal to 1:16, less than or equal to 1:15, less than or equal to 1:14, less than or equal to 1:13, less than or equal to 1:12, or less than or equal to 1:11.

Also provided herein is a method of preparing of a conjugate of Formula (B1):

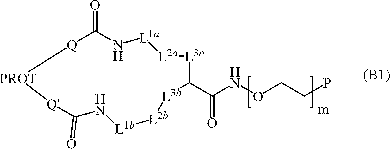

or a stereoisomer, a mixture of two or more diastereomers, a tautomer, or a mixture of two or more tautomers thereof; or a pharmaceutically acceptable salt, solvate, or hydrate thereof; comprising reacting (1) a compound of Formula (II):

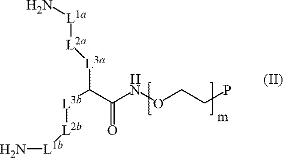

or a stereoisomer, a mixture of two or more diastereomers, a tautomer, or a mixture of two or more tautomers thereof; or a pharmaceutically acceptable salt, solvate, or hydrate thereof; and (2) a protein comprising two or more glutamine residues in the presence of a transglutaminase,wherein:
Q and Q′ are each a glutamine residue, wherein each glutamine residue independently is part of the sequence of PROT or is part of a Q-tag peptide sequence attached to PROT;
PROT is a protein connected to the rest of the conjugate via Q and Q′;
L^{1a} and L^{1b} are independently unsubstituted or substituted alkyl,
L^{2a} and L^{2b} are independently absent, unsubstituted or substituted alkyl, unsubstituted or substituted aryl, or unsubstituted or substituted heteroaryl,
L^{3a} and L^{3b} are independently absent or a linker moiety,
m is an integer selected from the group consisting of 0-50, and
P is an immunomodulating oligonucleotide.

In some embodiments, the ratio of compound (II) and the protein is in the range of 1:1 to 1:10 by molarity. In certain embodiments wherein the protein is an antibody, the ratio of compound (II) and the antibody is in the range of 1:1 to 1:10 by molarity.

In some embodiments, the molar ratio of the protein-to-transglutaminase is in the range of 1:5 to 1:20. In certain embodiments, the molar ratio of the protein-to-transglutaminase is in the range of 1:10 to 1:18. In other embodiments, the molar ratio of the protein-to-transglutaminase is at least 1:5, at least 1:6, at least 1:7, at least 1:8, at least 1:9, at least 1:10, at least 1:11, at least 1:12, at least 1:13, at least 1:14, at least 1:15, at least 1:16, or at least 1:17. In yet other embodiments, the molar ratio of the protein-to-transglutaminase is less than or equal to 1:20, less than or equal to 1:19, less than or equal to 1:18, less than or equal to 1:17, less than or equal to 1:17, less than or equal to 1:16, less than or equal to 1:15, less than or equal to 1:14, less than or equal to 1:13, less than or equal to 1:12, or less than or equal to 1:11.

Also provided herein is a compound of Formula (III):

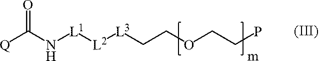

or a stereoisomer, a mixture of two or more diastereomers, a tautomer, or a mixture of two or more tautomers thereof; or a pharmaceutically acceptable salt, solvate, or hydrate thereof; wherein:
Q is a glutamine residue, wherein the glutamine residue is part of a Q-tag peptide sequence;
L¹ is unsubstituted or substituted alkyl,
L² is unsubstituted or substituted aryl, or unsubstituted or substituted heteroaryl,
L³ is absent or a linker moiety,
m is an integer selected from the group consisting of 0-50, and
P is an immunomodulating oligonucleotide.

Also provided herein is a pharmaceutical composition comprising a conjugate of Formula (A), (A1), (A2), (B) or (B1), an immunomodulating polynucleotide of Formula (I) or (II), or a compound of Formula (III) or a salt, solvate or stereoisomer of any of the foregoing and a pharmaceutically acceptable diluent, carrier or excipient. Also provided herein is a kit comprising a conjugate of Formula (A), (A1), (A2), (B) or (B1), or a salt, solvate or stereoisomer thereof. In some embodiments, the kit comprises a package insert containing instructions regarding indications, usage, dosage, administration, contraindications, other medicaments to be combined with the packaged product, and/or warnings concerning the use of such medicaments.

Also provided herein is a method for treating, preventing, or ameliorating one or more symptoms of a proliferative disease in a subject, comprising administering to the subject any conjugates or pharmaceutical compositions described herein. In some embodiments, the proliferative disease is cancer. Also provided herein is a method for modulating a natural killer cell in a subject, comprising administering to the subject any conjugates or pharmaceutical compositions described herein.

It is to be understood that one, some, or all of the properties of the various embodiments described herein may be combined to form other embodiments of the present invention. These and other embodiments of the invention are further described by the detailed description that follows.

### BRIEF DESCRIPTION OF THE DRAWINGS

FIGS. 1A-1E show the chromatograms of the conjugation reactions with Q-tag sequence WALQRPHYSYPD (SEQ ID NO: 16) and five linkers (L1, L2, L3, L4 and L5).

FIG. 2 shows percentage yields of transglutaminase-mediated conjugations (at a 500:1 ratio of NH₂:mTgase) of various Q-tags (SEQ ID NO: 6, 16, and 18) with linkers L2, L6, L7, L8, and L9.

FIG. 3 shows percentage yields of transglutaminase-mediated conjugations (at a 5000:1 ratio of NH₂:mTgase) of various Q-tags (SEQ ID NO: 6, 16, 17, and 18) with linkers L2, L6, and L10.

FIG. 4 shows percentage yields of transglutaminase-mediated conjugations of various Q-tags (SEQ ID NO: 6, 16, and 18) with linkers L2, L7, L8, L9, L11, and L12.

FIG. 5 shows percentage yields of transglutaminase-mediated conjugations of Q-tag WALQGPYTLTES (SEQ ID NO: 18) with various linkers (L2, L8-L9, L12-L28).

FIG. 6 shows percentage yields of transglutaminase-mediated conjugations of Q-tag WALQGPYTLTES (SEQ ID NO: 18) with various linkers (L2, L22-L24, L27, L29-L34).

FIG. 7 shows percentage yields of transglutaminase-mediated conjugations of Q-tag WALQGPYTLTES (SEQ ID NO: 18) with various linkers (L2, L12, L15-L16, L19, L22-L25, L27, L29, L35-L36).

FIG. 8 shows the percentage of conjugates remaining intact as a function of time (i.e., relative deconjugation), after initial conjugation (100%). The conjugates containing linkers with or without benzylamine moiety (linkers L13 and L37) and Q-tag peptide sequences LSLSPGLLQGG or WPAQGPT (SEQ ID NO: 7 or 8).

FIG. 9 shows the percentage of conjugates remaining intact as a function of time (i.e., relative deconjugation), after initial conjugation (100%), for Q-tag sequence LSLSPGLLQGG (SEQ ID NO: 7) conjugated to benzylamine linker L38 and L-amino acid-benzylamine linkers (L41, L42, and L44).

FIG. 10 shows the percentage of conjugates remaining intact as a function of time (i.e., relative deconjugation), after initial conjugation (100%), for Q-tag sequence WPAQGPT (SEQ ID NO: 8) conjugated to benzylamine linker L38 and L-amino acid-benzylamine linkers (L41, L42, and L44).

FIGS. 11A and 11B show the time-dependent yields of conjugates having one or two linkers (L38, L44, L46, and L49) attached to an anti-CD22 antibody comprising two Q-tag sequences. FIG. 11A shows the yield for one linker attached to the one antibody (DAR 1); FIG. 11B shows the yield for two linkers attached to the one antibody (DAR 2) (FIG. 11B).

FIGS. 12A and 12B show conjugation a linker containing two nucleophile sites (i.e., —NH₂) with the heavy chain of an antibody, and the production of cross-linked and non-cross linked conjugates.

FIG. 13 shows the percentage yields of transglutaminase-mediated conjugations with various linkers (L39, L42, and L44) and Q-tags (SEQ ID NO: 7-15).

FIGS. 14A-14B show the percentage of oligonucleotide-Q-tag conjugate observed over time in transglutaminase for oligonucleotide (tsuscsgstscsgstsgsascsgstst-c3, SEQ ID NO: 5), Q-tag sequence LSLSPGLLQGG (SEQ ID NO: 7) and linkers L51 (in FIG. 14A) and L39 (in FIG. 14B).

FIGS. 15A-15B show conjugation of linker L49 containing two nucleophile sites (i.e., —NH₂) with the heavy chain of an antibody, and the production of cross-linked and non-cross linked conjugates.

### DETAILED DESCRIPTION

The present description is based on the discovery that certain polypeptide-oligonucleotide conjugates provide enhanced stability and delivery selectivity. The description also provides the methods for preparing these conjugates. Particularly, the conjugation can be performed by a transglutaminase (Tgase)-mediated reaction. The description also provides intermediate compounds that can be used to prepare these conjugates as well as compositions and kits that contain these polypeptide-oligonucleotide conjugates.

### I. Definitions

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Throughout this application, unless the context indicates otherwise, references to a conjugate of Formula (A), (A1), (A2), (B) or (B1),or a compound of Formula (I), (II), or (III), include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. In some embodiments, references to a conjugate of Formula (A), (A1), (A2), (B) or (B1), or a compound of Formula (I), (II), or (III), include polymorphs, solvates, co-crystals, isomers, tautomers and/or oxides thereof. In some embodiments, references to a conjugate of Formula (A), (A1), (A2), (B) or (B1), or a compound of Formula (I), (II), or (III), include polymorphs, solvates, and/or co-crystals thereof. In some embodiments, references to references to a conjugate of Formula (A), (A1), (A2), (B) or (B1), or a compound of Formula (I), (II), or (III), include isomers, tautomers and/or oxides thereof. In some embodiments, references to references to a conjugate of Formula (A), (A1), (A2), (B) or (B1), or a compound of Formula (I), (II), or (III), include solvates thereof.

“Alkyl” encompasses straight and branched carbon chains having the indicated number of carbon atoms, for example, from 1 to 20 carbon atoms, or 1 to 8 carbon atoms, or 1 to 6 carbon atoms. For example, C₁₋₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “propyl” includes n-propyl and isopropyl; and “butyl” includes n-butyl, sec-butyl, isobutyl and t-butyl. Examples of alkyl groups include, but are not limited to, methyl, ethyl, propyl, isopropyl, n-butyl, sec-butyl, tert-butyl, pentyl, 2-pentyl, 3-pentyl, isopentyl, neopentyl, hexyl, 2-hexyl, 3-hexyl, and 3-methylpentyl.

When a range of values is given (e.g., C₁₋₆ alkyl), each value within the range as well as all intervening ranges are included. For example, “C₁₋₆ alkyl” includes C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

The term “amino,” as used herein, represents —N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —COOR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., —NHR^{N1}), where R^{N1} is independently —OH, —SO₂OR^{N2}, —SO₂R^{N2}, —SOR^{N2} —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In certain embodiments, an amino group is —NHR^{N1}, in which R^{N1} is optionally substituted alkyl. Non-limiting examples of —NHR^{N1}, in which R^{N1} is optionally substituted alkyl, include: optionally substituted alkylamino, a proteinogenic amino acid, a non-proteinogenic amino acid, a C₁₋₆ alkyl ester of a proteinogenic amino acid, and a C₁₋₆ alkyl ester of a non-proteinogenic amino acid. The amino acid employed is optionally in the L-form.

The term “immunomodulating polynucleotide” as used herein, represents a polynucleotide construct containing a total of from 6 to 50 contiguous nucleosides covalently bound together by internucleoside bridging groups independently selected from the group consisting of internucleoside phosphoesters and optionally internucleoside abasic spacers. The immunomodulating polynucleotides are capped at 5′- and 3′- termini with 5′- and 3′-capping groups, respectively. The immunomodulating polynucleotides are capable of modulating an innate immune response, as determined by, e.g., a change in the activation of intracellular signaling pathway(s) including but not limited to NFκB, a change in the expression of an activation marker or a change in the secretion of at least one inflammatory cytokine or at least one type I interferon in an immune cell (e.g., antigen-presenting cell) to which an immunomodulating polynucleotide was delivered (e.g., in comparison to another immune cell (e.g., antigen-presenting cell) to which an immunomodulating polynucleotide was not delivered) or in an immune cell that interacts with an immune cell (e.g., antigen-presenting cell) to which an immunomodulating polynucleotide was delivered (including direct cell-to-cell interactions as well as indirect stimulation, e.g., from one or more cytokines secreted by the cell to which an immunomodulating polynucleotide was delivered). The immunomodulating polynucleotide may contain a conjugating group or, if the immunomodulating polynucleotide is part of a conjugate, a linker bonded to a targeting moiety and optionally to one or more (e.g., 1 to 6) auxiliary moieties (e.g., polyethylene glycols). The conjugating group or the linker may be part of the phosphotriester or the terminal capping group.

The term “immunostimulating polynucleotide” as used herein, represents an immunomodulating polynucleotide capable of activating an immune response, as determined by, e.g., an increase in the activation of intracellular signaling pathway(s) such asNFκB or an increase in levels of cell surface markers) of activation or function or an increase in the secretion of at least one inflammatory cytokine or at least one type I interferon in an immune cell (e.g., antigen-presenting cell) to which an immunostimulating polynucleotide was delivered (e.g., in comparison to another immune cell (e.g., antigen-presenting cell) to which an immunostimulating polynucleotide was not delivered) or in an immune cell that interacts with an immune cell (e.g., antigen-presenting cell) to which an immunomodulating polynucleotide was delivered (including direct cell-to-cell interactions as well as indirect stimulation, e.g., from one or more cytokines secreted by the cell to which an immunomodulating polynucleotide was delivered). In some embodiments, the immunostimulating polynucleotide contains at least one cytidine-p-guanosine (CpG) sequence, in which p is an internucleoside phosphodiester (e.g., phosphate or phosphorothioate) or an internucleoside phosphotriester or phosphothiotriester. As used herein, the CpG-containing immunostimulating polynucleotide can be naturally existing, such as CpG ODNs of bacterial or viral origins, or synthetic. For example, in some embodiments, the CpG sequence in the immunostimulating polynucleotide contains 2′-deoxyribose. In some embodiments, the CpG sequence in the immunostimulating polynucleotide is unmethylated.

The term “immunosuppressive polynucleotide” as used herein, represents an immunomodulating polynucleotide capable of antagonizing an immune response, as determined by e.g., a reduction in the activation or lack of activation of NFκB or lack of increase in the levels of cell surface marker(s) of activation of function or a reduction or lack of increase in the secretion of at least one inflammatory cytokine or at least one type I interferon in an immune cell (e.g., antigen-presenting cell) to which an immunosuppressive polynucleotide was delivered (e.g., in comparison to another immune cell (e.g., antigen-presenting cell) to which an immunosuppressive polynucleotide was not delivered) or in an immune cell that interacts with an immune cell (e.g., antigen-presenting cell) to which an immunomodulating polynucleotide was delivered (including direct cell-to-cell interactions as well as indirect stimulation, e.g., from one or more cytokines secreted by the cell to which an immunomodulating polynucleotide was delivered).

It is to be understood that the terms “immunomodulating polynucleotide,” “immunostimulating polynucleotide,” “immunosuppressive polynucleotide,” and “conjugate” encompass salts of the immunomodulating polynucleotide, immunostimulating polynucleotide, immunosuppressive polynucleotide and conjugate, respectively. For example, the terms “immunomodulating polynucleotide,” “immunostimulating polynucleotide,” “immunosuppressive polynucleotide,” and “conjugate” encompasses both the protonated, neutral form (P-XH moiety, where X is O or S) of a phosphate, phosphorothioate, or phosphorodithioate and the deprotonated, ionic form (P-X⁻ moiety, where X is O or S) of a phosphate, phosphorothioate, or phosphorodithioate. Accordingly, it is to be understood that the phosphoesters and phosphodiesters described as having one or more of R^{E1,} R^{E2,} and R^{E3} as hydrogen encompass salts, in which the phosphate, phosphorothioate, or phosphorodithioate is present in a deprotonated, ionic form.

The term “phosphotriester,” as used herein, refers to a phosphoester, in which all three valences are substituted with non-hydrogen substituents. The phosphotriester consists of phosphate, phosphorothioate, or phosphorodithioate; one or two bonds to nucleoside(s), or abasic spacer(s), and/or phosphoryl group(s); and one or two groups independently selected from the group consisting of a bioreversible group; a non-bioreversible group; an auxiliary moiety; a conjugating group; and a linker bonded to a targeting moiety and optionally to one or more (e.g., 1 to 6) auxiliary moieties. A terminal phosphotriester includes one bond to a group containing a nucleoside and two groups independently selected from the group consisting of a bioreversible group; a non-bioreversible group; an auxiliary moiety; a conjugating group; a phosphoryl group; and a linker bonded to a targeting moiety and optionally to one or more (e.g., 1 to 6) auxiliary moieties. In some embodiments, a terminal phosphotriester contains 1 or 0 linkers bonded to a targeting moiety and optionally to one or more (e.g., 1 to 6) auxiliary moieties. An internucleoside phosphotriester includes two bonds to nucleoside-containing groups. A phosphotriester may be a group of the following structure:

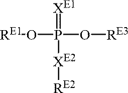

wherein:
each of X^{E1} and X^{E2} is independently O or S;
each or R^{E1} and R^{E3} is independently a bond to a nucleoside; a sugar analogue of an abasic spacer; a bioreversible group; a non-bioreversible group; an auxiliary moiety; a conjugating group; a linker bonded to a targeting moiety; a linker bonded to a targeting moiety and one or more (e.g., 1 to 6) auxiliary moieties; or the phosphorus atom in a group of formula —P(═X^{E1})(—X^{E2}—R^{E2}A)—O—,
   where R^{E2}A is hydrogen; a bioreversible group; a non-bioreversible group; an auxiliary moiety; a conjugating group; a linker bonded to a targeting moiety; or a linker bonded to a targeting moiety and one or more (e.g., 1 to 6) auxiliary moieties; and
R^{E2} is a bioreversible group; a non-bioreversible group; an auxiliary moiety; a conjugating group; a linker bonded to a targeting moiety; or a linker bonded to a targeting moiety and one or more (e.g., 1 to 6) auxiliary moieties;
provided that at least one of R^{E1} and R^{E3} is a bond to a group containing at least one nucleoside.
If both R^{E1} and R^{E3} are bonds to groups containing at least one nucleoside, the phosphotriester is an internucleoside phosphotriester. If one and only one of R^{E1} and R^{E3} is a bond to a group containing a nucleoside, the phosphotriester is a terminal phosphotriester.

As used herein, the term “amino acid” refers to any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, omithine, hypusine, 2-aminoisobutyric acid, dehydroalanine, gamma-aminobutyric acid, citrulline, beta-alanine, alpha-ethyl-glycine, alpha-propyl-glycine and norleucine.

The terms “antibody,” “immunoglobulin,” and “Ig” are used interchangeably herein, and are used in the broadest sense and specifically cover, for example, individual monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies. An antibody can be human, humanized, chimeric and/or affinity matured as well as an antibody from other species, for example, mouse and rabbit.

The term “antibody” is intended to include a polypeptide product of B cells within the immunoglobulin class of polypeptides that is able to bind to a specific antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxyl-terminal portion of each chain includes a constant region. See Borrebaeck (ed.) (1995) Antibody Engineering, Second Ed., Oxford University Press.; Kuby (1997) Immunology, Third Ed., W.H. Freeman and Company, New York. Antibodies also include, but are not limited to, synthetic antibodies, monoclonal antibodies, recombinant antibodies, multispecific antibodies (including bi-specific antibodies), human antibodies, humanized antibodies, camelized antibodies, chimeric antibodies, intrabodies, anti-idiotypic (anti-Id) antibodies, and functional fragments thereof, which refers a portion of an antibody heavy or light chain polypeptide that retains some or all of the binding activity of the antibody from which the fragment is derived. Non-limiting examples of functional fragments of an antibody include single-chain Fvs (scFv) (e.g., including monospecific or bispecific), Fab fragments, F(ab′) fragments, F(ab)₂ fragments, F(ab′)₂ fragments, disulfide-linked Fvs (sdFv), Fd fragments, Fv fragments, scRv-Fc, nanobody, diabody, triabody, tetrabody, and minibody. In some embodiments, the antibody comprises an Fc variant that has reduced or ablated effector function. In particular, antibodies provided herein include immunoglobulin molecules and immunologically active portions of immunoglobulin molecules, for example, antigen-binding domains or molecules that contain an antigen-binding site that binds to the antigen (e.g., one or more complementarity determining regions (CDRs) of an anti-CD56 antibody or an anti-SIRP-α antibody). Such antibody fragments are described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics 1993, 22, 189-224; Plückthun and Skerra, Meth. Enzymol. 1989, 178, 497-515; and Day, Advanced Immunochemistry, Second Ed., Wiley-Liss, Inc., New York, NY (1990). The antibodies provided herein can be of any type (e.g., IgG, IgE, IgM, IgD, IgA, and IgY), any class (e.g., IgG1, IgG2, IgG3, IgG4, IgA1, and IgA2), or any subclass (e.g., IgG2a and IgG2b) of an immunoglobulin molecule.

The term “antigen” refers to a predetermined target to which an antibody can selectively bind. A target antigen can be a polypeptide, carbohydrate, nucleic acid, lipid, hapten, or fragment thereof, or other naturally occurring or synthetic compound. In one embodiment, the target antigen is a polypeptide.

The terms “antigen-binding fragment,” “antigen-binding domain,” and “antigen-binding region” refer to a portion of an antibody that comprises the amino acid residues that interact with an antigen (e.g., a polypeptide, carbohydrate, nucleic acid, lipid, hapten, or fragment thereof, or other naturally-occurring or synthetic compound) and confer on the binding agent its specificity and affinity for the antigen (e.g., complementarity determining regions (CDRs)).

The term “specific binding,” “specifically binds to,” or “specific for” a particular polypeptide or an epitope on a particular polypeptide target can be exhibited, for example, by a molecule (e.g., an antibody) having a dissociation constant (K_{d}) for the target of at least about 10⁻⁴ M, at least about 10⁻⁵ M, at least about 10⁻⁶ M, at least about 10⁻⁷ M, at least about 10⁻⁸ M, at least about 10⁻⁹ M, at least about 10⁻¹⁰ M, at least about 10⁻¹¹ M, or at least about 10⁻¹² M. In one embodiment, the term “specific binding” refers to binding where a molecule binds to a particular polypeptide or epitope on a particular polypeptide without substantially binding to any other polypeptide or polypeptide epitope.

A 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for µ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8^{th} edition, Stites et al. (eds.), Appleton & Lange, Norwalk, CT, 1994, page 71 and Chapter 6.

The term “variable region” or “variable domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen-binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5^{th} Ed. Public Health Service, National Institutes of Health, Bethesda, MD, 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “variable region residue numbering as in Kabat” or “amino acid position numbering as in Kabat”, and variations thereof, refers to the numbering system used for heavy chain variable regions or light chain variable regions of the compilation of antibodies in Kabat et al., Sequences of Proteins of Immunological Interest, 5^{th} Ed. Public Health Service, National Institutes of Health, Bethesda, MD. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc, according to Kabat) after heavy chain FR residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., Sequences of Immunological Interest. 5^{th} Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, including, for example, by AbM, Chothia, Contact, IMGT and Ahon.

An “intact” antibody is one comprising an antigen-binding site as well as a CL and at least heavy chain constant regions, CH1, CH2 and CH3. The constant regions may include human constant regions or amino acid sequence variants thereof. Preferably, an intact antibody has one or more effector functions.

The term “antibody fragment” refers to a portion of an intact antibody, preferably the antigen-binding or variable region of the intact antibody. Examples of antibody fragments include, without limitation, Fab, Fab′, F(ab′)2, and Fv fragments; diabodies and di-diabodies (see, e.g., Holliger et al., Proc. Natl. Acad. Sci. U.S.A.1993, 90, 6444-8; Lu et al., J. Biol. Chem. 2005, 280, 19665-72; Hudson et al., Nat. Med. 2003, 9, 129-134; WO 93/11161; and U.S. Pat. Nos. 5,837,242 and 6,492,123); single-chain antibody molecules (see, e.g., U.S. Pat. Nos. 4,946,778; 5,260,203; 5,482,858 and 5,476,786); dual variable domain antibodies (see, e.g., U.S. Pat. No. 7,612,181); single variable domain antibodies (SdAbs) (see, e.g., Woolven et al., Immunogenetics 1999, 50, 98-101 Streltsov et al., Proc. Natl. Acad. Sci. U.S.A.2004, 101, 12444-12449); and multispecific antibodies formed from antibody fragments.

The term “functionalfragment,” “binding fragment,” or “antigen-binding fragment” of an antibody refers to a molecule that exhibits at least one of the biological functions attributed to the intact antibody, the function comprising at least binding to the target antigen.

The term “heavy chain” when used in reference to an antibody refers to a polypeptide chain of about 50-70 kDa, wherein the amino-terminal portion includes a variable region of about 120 to 130 or more amino acids and a carboxyl-terminal portion that includes a constant region. The constant region can be one of five distinct types, (e.g., isotypes) referred to as alpha (α), delta (δ), epsilon (ε), gamma (γ) and mu (µ), based on the amino acid sequence of the heavy chain constant region. The distinct heavy chains differ in size: α, δ and γ contain approximately 450 amino acids, while µ and ε contain approximately 550 amino acids. When combined with a light chain, these distinct types of heavy chains give rise to five well known classes (e.g., isotypes) of antibodies, IgA, IgD, IgE, IgG and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxyl-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only a beta klotho epitope as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., Nature 1975, 256, 495; or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature 1991, 352, 624-628 and Marks et al., J. Mol. Biol. 1991, 222, 581-597, for example. Other methods for the preparation of clonal cell lines and of monoclonal antibodies expressed thereby are well known in the art (see, for example, Chapter 11 in: Short Protocols in Molecular Biology, (2002) 5^{th} Ed., Ausubel et al., eds., John Wiley and Sons, New York). Exemplary methods of producing monoclonal antibodies are provided in the Examples herein.

“Humanized” forms of nonhuman (e.g., murine) antibodies are chimeric antibodies that include human immunoglobulins (e.g., recipient antibody) in which the native CDR residues are replaced by residues from the corresponding CDR of a nonhuman species (e.g., donor antibody) such as mouse, rat, rabbit or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 1986, 321, 522-525; Riechmann et al., Nature 1988, 332, 323-329; Presta, Curr. Opin. Biotechnol. 1992, 3, 394-398; Carter et al., Proc. Natl. Acad. Sci. U.S.A.1992, 89, 4285-4289; and U.S. Pat. Nos: 6,800,738, 6,719,971, 6,639,055, 6,407,213, and 6,054,297.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 1991, 227, 381; Marks et al., J. Mol. Biol. 1991, 222, 581) and yeast display libraries (Chao et al., Nature Protocols 2006, 1, 755-768). Also available for the preparation of human monoclonal antibodies are methods described in Cole et al., Monoclonal Antibodies and Cancer Therapy, Alan R. Liss, p. 77 (1985); Boerner et al., J. Immunol. 1991, 147, 86-95. See also van Dijk and van de Winkel, Curr. Opin. Pharmacol. 2001, 5, 368-374. Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 1995, 6, 561-566; Brüggemann and Taussing, Curr. Opin. Biotechnol. 1997, 8, 455-458; and U.S. Pat. Nos. 6,075,181 and 6,150,584 regarding XENOMOUSE™ technology). See also, for example, Li et al., Proc. Natl. Acad. Sci. U.S.A.2006, 103, 3557-3562 regarding human antibodies generated via a human B-cell hybridoma technology.

A “CDR” refers to one of three hypervariable regions (H1, H2, or H3) within the non-framework region of the immunoglobulin (Ig or antibody) VH β-sheet framework, or one of three hypervariable regions (L1, L2, or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains. Kabat et al., J. Biol. Chem. 1977, 252, 6609-6616; Kabat, Adv. Protein Chem. 1978, 32, 1-75. CDR region sequences also have been defined structurally by Chothia as those residues that are not part of the conserved β-sheet framework, and thus are able to adapt different conformations. Chothia and Lesk, J. Mol. Biol. 1987, 196, 901-917. Both terminologies are well recognized in the art. CDR region sequences have also been defined by AbM, Contact and IMGT. The positions of CDRs within a canonical antibody variable region have been determined by comparison of numerous structures. Al-Lazikani et al., J. Mol. Biol. 1997, 273, 927-948; Morea et al., Methods. 2000, 20, 267-279. Because the number of residues within a hypervariable region varies in different antibodies, additional residues relative to the canonical positions are conventionally numbered with a, b, c and so forth next to the residue number in the canonical variable region numbering scheme. Al-Lazikani et al., supra (1997). Such nomenclature is similarly well known to those skilled in the art.

The term “hypervariable region”, “HVR”, or “HV”, when used herein refers to the regions of an antibody variable region that are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (see, e.g., Kabat et al., Sequences of Proteins of Immunological Interest, 5^{th} Ed. Public Health Service, National Institutes of Health, Bethesda, MD. (1991)). Chothia refers instead to the location of the structural loops. See, e.g., Chothia and Lesk, J. Mol. Biol. 1987, 196, 901-917. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular’s AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

“Cycloalkyl” indicates a non-aromatic, fully saturated carbocyclic ring having the indicated number of carbon atoms, for example, 3 to 10, or 3 to 8, or 3 to 6 ring carbon atoms. Cycloalkyl groups may be monocyclic or polycyclic (e.g., bicyclic, tricyclic). Examples of cycloalkyl groups include cyclopropyl, cyclobutyl, cyclopentyl, and cyclohexyl, as well as bridged and caged ring groups (e.g., norbornane, bicyclo[2.2.2]octane). In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group. Examples of polycyclic cycloalkyl groups consisting of a cycloalkyl group fused to an aromatic ring are described below.

“Cycloalkenyl” indicates a non-aromatic carbocyclic ring, containing the indicated number of carbon atoms (e.g., 3 to 10, or 3 to 8, or 3 to 6 ring carbon atoms) and at least one carbon-carbon double bond. Cycloalkenyl groups may be monocyclic or polycyclic (e.g., bicyclic, tricyclic). Examples of cycloalkenyl groups include cyclopropenyl, cyclobutenyl, cyclopentenyl, cyclopentadienyl, and cyclohexenyl, as well as bridged and caged ring groups (e.g., bicyclo[2.2.2]octene). In addition, one ring of a polycyclic cycloalkenyl group may be aromatic, provided the polycyclic alkenyl group is bound to the parent structure via a non-aromatic carbon atom. For example, inden-1-yl (wherein the moiety is bound to the parent structure via a non-aromatic carbon atom) is considered a cycloalkenyl group, while inden-4-yl (wherein the moiety is bound to the parent structure via an aromatic carbon atom) is not considered a cycloalkenyl group. Examples of polycyclic cycloalkenyl groups consisting of a cycloalkenyl group fused to an aromatic ring are described below.

“Cycloalkynyl” refers to an unsaturated hydrocarbon group within a cycloalkyl having at least one site of acetylenic unsaturation (i.e., having at least one moiety of the formula C═C). Cycloalkynyl can consist of one ring, such as cyclooctyne, or multiple rings. One cycloalkynyl moiety is an unsaturated cyclic hydrocarbon having from 5 to 10 annular carbon atoms (a “C₅-C₁₀ cycloalkynyl”). Examples include cyclopentyne, cyclohexyne, cycloheptyne, cyclooctyne, cyclononyne, and the like.

“Aryl” indicates an aromatic carbocyclic ring having the indicated number of carbon atoms, for example, 6 to 12 or 6 to 10 carbon atoms. Aryl groups may be monocyclic or polycyclic (e.g., bicyclic, tricyclic). In some instances, both rings of a polycyclic aryl group are aromatic (e.g., naphthyl). In other instances, polycyclic aryl groups may include a non-aromatic ring fused to an aromatic ring, provided the polycyclic aryl group is bound to the parent structure via an atom in the aromatic ring. Thus, a 1,2,3,4-tetrahydronaphthalen-5-yl group (wherein the moiety is bound to the parent structure via an aromatic carbon atom) is considered an aryl group, while 1,2,3,4-tetrahydronaphthalen-1-yl (wherein the moiety is bound to the parent structure via a non-aromatic carbon atom) is not considered an aryl group. Similarly, a 1,2,3,4-tetrahydroquinolin-8-yl group (wherein the moiety is bound to the parent structure via an aromatic carbon atom) is considered an aryl group, while 1,2,3,4-tetrahydroquinolin-1-yl group (wherein the moiety is bound to the parent structure via a non-aromatic nitrogen atom) is not considered an aryl group. However, the term “aryl” does not encompass or overlap with “heteroaryl”, as defined herein, regardless of the point of attachment (e.g., both quinolin-5-yl and quinolin-2-yl are heteroaryl groups). In some instances, aryl is phenyl or naphthyl. In certain instances, aryl is phenyl. Additional examples of aryl groups comprising an aromatic carbon ring fused to a non-aromatic ring are described below.

The term “DAR” refers to a drug-antibody ratio of an oligonucleotide-antibody conjugate, more specifically an immunomodulating polynucleotide-antibody ratio.

“Heteroaryl” indicates an aromatic ring containing the indicated number of atoms (e.g., 5 to 12, or 5 to 10 membered heteroaryl) made up of one or more heteroatoms (e.g., 1, 2, 3 or 4 heteroatoms) selected from N, O and S and with the remaining ring atoms being carbon. Heteroaryl groups do not contain adjacent S and O atoms. In some embodiments, the total number of S and O atoms in the heteroaryl group is not more than 2. In some embodiments, the total number of S and O atoms in the heteroaryl group is not more than 1. Unless otherwise indicated, heteroaryl groups may be bound to the parent structure by a carbon or nitrogen atom, as valency permits. For example, “pyridyl” includes 2-pyridyl, 3-pyridyl and 4-pyridyl groups, and “pyrrolyl” includes 1-pyrrolyl, 2-pyrrolyl and 3-pyrrolyl groups.

In some instances, a heteroaryl group is monocyclic. Examples include pyrrole, pyrazole, imidazole, triazole (e.g., 1,2,3-triazole, 1,2,4-triazole, 1,2,4-triazole), tetrazole, furan, isoxazole, oxazole, oxadiazole (e.g., 1,2,3-oxadiazole, 1,2,4-oxadiazole, 1,3,4-oxadiazole), thiophene, isothiazole, thiazole, thiadiazole (e.g., 1,2,3-thiadiazole, 1,2,4-thiadiazole, 1,3,4-thiadiazole), pyridine, pyridazine, pyrimidine, pyrazine, triazine (e.g., 1,2,4-triazine, 1,3,5-triazine) and tetrazine.

In some instances, both rings of a polycyclic heteroaryl group are aromatic. Examples include indole, isoindole, indazole, benzoimidazole, benzotriazole, benzofuran, benzoxazole, benzoisoxazole, benzoxadiazole, benzothiophene, benzothiazole, benzoisothiazole, benzothiadiazole, 1H-pyrrolo[2,3-b]pyridine, 1H-pyrazolo[3,4-b]pyridine, 3H-imidazo[4,5-b]pyridine, 3H-[1,2,3]triazolo[4,5-b]pyridine, 1H-pyrrolo[3,2-b]pyridine, 1H-pyrazolo[4,3-b]pyridine, 1H-imidazo[4,5-b]pyridine, 1H-[1,2,3]triazolo[4,5-b]pyridine, 1H-pyrrolo[2,3-c]pyridine, 1H-pyrazolo[3,4-c]pyridine, 3H-imidazo[4,5-c]pyridine, 3H-[1,2,3]triazolo[4,5-c]pyridine, 1H-pyrrolo[3,2-c]pyridine, 1H-pyrazolo[4,3-c]pyridine, 1H-imidazo[4,5-c]pyridine, 1H-[1,2,3]triazolo[4,5-c]pyridine, furo[2,3-b]pyridine, oxazolo[5,4-b]pyridine, isoxazolo[5,4-b]pyridine, [1,2,3]oxadiazolo[5,4-b]pyridine, furo[3,2-b]pyridine, oxazolo[4,5-b]pyridine, isoxazolo[4,5-b]pyridine, [1,2,3]oxadiazolo[4,5-b]pyridine, furo[2,3-c]pyridine, oxazolo[5,4-c]pyridine, isoxazolo[5,4-c]pyridine, [1,2,3]oxadiazolo[5,4-c]pyridine, furo[3,2-c]pyridine, oxazolo[4,5-c]pyridine, isoxazolo[4,5-c]pyridine, [1,2,3]oxadiazolo[4,5-c]pyridine, thieno[2,3-b]pyridine, thiazolo[5,4-b]pyridine, isothiazolo[5,4-b]pyridine, [1,2,3]thiadiazolo[5,4-b]pyridine, thieno[3,2-b]pyridine, thiazolo[4,5-b]pyridine, isothiazolo[4,5-b]pyridine, [1,2,3]thiadiazolo[4,5-b]pyridine, thieno[2,3-c]pyridine, thiazolo[5,4-c]pyridine, isothiazolo[5,4-c]pyridine, [1,2,3]thiadiazolo[5,4-c]pyridine, thieno[3,2-c]pyridine, thiazolo[4,5-c]pyridine, isothiazolo[4,5-c]pyridine, [1,2,3]thiadiazolo[4,5-c]pyridine, quinoline, isoquinoline, cinnoline, quinazoline, quinoxaline, phthalazine, naphthyridine (e.g., 1,8-naphthyridine, 1,7-naphthyridine, 1,6-naphthyridine, 1,5-naphthyridine, 2,7-naphthyridine, 2,6-naphthyridine), imidazo[1,2-a]pyridine, 1H-pyrazolo[3,4-d]thiazole, 1H-pyrazolo[4,3-d]thiazole and imidazo[2,1-b]thiazole.

In other instances, polycyclic heteroaryl groups may include a non-aromatic ring (e.g., cycloalkyl, cycloalkenyl, heterocycloalkyl, heterocycloalkenyl) fused to a heteroaryl ring, provided the polycyclic heteroaryl group is bound to the parent structure via an atom in the aromatic ring. For example, a 4,5,6,7-tetrahydrobenzo[d]thiazol-2-yl group (wherein the moiety is bound to the parent structure via an aromatic carbon atom) is considered a heteroaryl group, while 4,5,6,7-tetrahydrobenzo[d]thiazol-5-yl (wherein the moiety is bound to the parent structure via a non-aromatic carbon atom) is not considered a heteroaryl group. Examples of polycyclic heteroaryl groups consisting of a heteroaryl ring fused to a non-aromatic ring are described below.

As used herein, the terms “including,” “containing,” and “comprising” are used in their open, non-limiting sense. It is also understood that aspects and embodiments of the invention described herein may include “consisting” and/or “consisting essentially of” aspects and embodiments.

It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

As used herein, a “carrier” includes pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “effective amount” or “therapeutically effective amount” of a substance is at least the minimum concentration required to bring about a measurable improvement or prevention of a particular disorder. An effective amount herein may vary according to factors such as the disease state, age, sex, and weight of the patient, and the ability of the substance to elicit a desired response in the individual. An effective amount is also one in which any toxic or detrimental effects of the treatment are outweighed by the therapeutically beneficial effects. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

The terms “protein”, “polypeptide” and “peptide” are used herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Typically, a protein for use herein will have a molecular weight of at least about 5-20 kDa, alternatively at least about 20-100 kDa, or at least about 100 kDa. Also included within the definition are, for example, proteins containing one or more analogs of an amino acid (including, for example, unnatural amino acids, etc.), as well as other modifications known in the art.

A “pharmaceutically acceptable salt” is a salt form that is non-toxic, biologically tolerable, or otherwise biologically suitable for administration to the subject. See generally Berge et al.(1977) J. Pharm. Sci. 66, 1. Particular pharmaceutically acceptable salts are those that are pharmacologically effective and suitable for contact with the tissues of subjects without undue toxicity, irritation, or allergic response. Pharmaceutically acceptable salts include, without limitation, acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like. These salts may be derived from inorganic or organic acids. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. In some embodiments, pharmaceutically acceptable salts are formed when an acidic proton present in the parent compound either is replaced by a metal ion, e.g., an alkali metal ion, an alkaline earth ion, or an aluminum ion; or coordinates with an organic base. Salts derived from pharmaceutically acceptable organic non-toxic bases include, without limitation, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, trimetharnine, dicyclohexylamine, caffeine, procaine, hydrabamine, choline, betaine, ethylenediamine, glucosamine, N- ethylglucamine, N-methylglucamine, theobromine, purines, piperazine, piperidine, N- ethylpiperidine, polyamine resins, amino acids such as lysine, arginine, histidine, and the like. Examples of pharmaceutically acceptable base addition salts include those derived from inorganic bases such as sodium, potassium, lithium, ammonium, calcium, magnesium, iron, zinc, copper, manganese, aluminum salts and the like. In some embodiments, the organic non-toxic bases are L-amino acids, such as L-lysine and L- arginine, tromethamine, N-ethylglucamine and N-methylglucamine. Acceptable inorganic bases include, without limitation, aluminum hydroxide, calcium hydroxide, potassium hydroxide, sodium carbonate, sodium hydroxide, and the like. Lists of other suitable pharmaceutically acceptable salts are found in Remington’s Pharmaceutical Sciences, 17^{th} Edition, Mack Publishing Company, Easton, Pa., 1985.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

A “subject,” “patient” or “individual” includes a mammal, such as a human or other animal, and typically is human. In some embodiments, the subject, e.g., patient, to whom the therapeutic agents and compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent, a dog, a cat, a farm animal, such as a cow or a horse, etc.

The term “cancer” or “tumor” refers to the presence of cells possessing characteristics typical of cancer-causing cells, such as uncontrolled proliferation, immortality, metastatic potential, rapid growth and proliferation rate, and certain characteristic morphological features. In some embodiments, such cells exhibit such characteristics in part or in full due to the expression and activity of immune checkpoint inhibitors, such as PD-1, PD-L1, and/or CTLA-4. Cancer cells are often in the form of a solid tumor, which is detectable on the basis of tumor mass, e.g., by procedures such as CAT scan, MR imaging, X-ray, ultrasound or palpation, and/or which is detectable because of the expression of one or more cancer-specific antigens in a sample obtainable from a patient. In some embodiments, a solid tumor does not need to have measurable dimensions. Cancer cells may also in the form of a liquid tumor, which cancer cells may exist alone or disseminated within an animal. As used herein, the terms “disseminated tumor” and “liquid tumor” are used interchangeably, and include, without limitation, leukemia and lymphoma and other blood cell cancers.

The term “leukemia” refers to a type of cancer of the blood or bone marrow characterized by an abnormal increase of immature white blood cells called “blasts.” Leukemia is a broad term covering a spectrum of diseases. In turn, it is part of the even broader group of diseases affecting the blood, bone marrow, and lymphoid system, which are all known as hematological neoplasms. Leukemias can be divided into four major classifications, acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia.

The term “lymphoma” refers to a group of blood cell tumors that develop from lymphatic cells. The two main categories of lymphomas are Hodgkin lymphomas (HL) and non-Hodgkin lymphomas (NHL) Lymphomas include any neoplasms of the lymphatic tissues. The main classes are cancers of the lymphocytes, a type of white blood cell that belongs to both the lymph and the blood and pervades both.

As used herein, the term “cancer” includes premalignant as well as malignant cancers, and also includes primary tumors (e.g., those whose cells have not migrated to sites in the subject’s body other than the site of the original tumor) and secondary tumors (e.g., those arising from metastasis, the migration of tumor cells to secondary sites that are different from the site of the original tumor), recurrent cancer and refractory cancer.

The terms “cancer recurrence” and “cancer relapse” are used interchangeably and refer to the return of a sign, symptom or disease after a remission. The recurrent cancer cells may re-appear in the same site of the primary tumor or in another location, such as in secondary cancer. The cancer cells may re-appear in the same diseased form as the primary cancer or a different diseased form. For example, in some embodiments, a primary cancer is a solid tumor, and the recurrent cancer is a liquid tumor. In other embodiments, a primary cancer is a liquid tumor, and the recurrent cancer is a solid tumor. In yet other embodiments, the primary cancer and the recurrent cancer are both solid tumors, or both liquid tumors. In some embodiments, the recurrent tumor expresses at least one tumor-associated antigen that is also expressed by the primary tumor.

The term “refractory cancer” as used herein refers to a cancer that does not respond to a treatment, for example, a cancer that is resistant at the beginning of treatment (e.g., treatment with an immunotherapy) or a cancer that may become resistant during treatment. The terms “respond,” “response” or “responsiveness” refer to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

As used herein, cancers include, but are not limited to, B cell cancer, e.g., multiple myeloma, Waldenström’s macroglobulinemia, the heavy chain diseases, such as, for example, alpha chain disease, gamma chain disease, and mu chain disease, benign monoclonal qammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms’ tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin’s disease and non-Hodgkin’s disease), multiple myeloma, Waldenstrom’s macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The term “cancer therapy” or “cancer therapeutic agent” as used herein, refers to those therapies or agents that can exert anti-tumor effect or have an anti-tumor activity. Such anti-tumor effect or anti-tumor activity can be exhibited as a reduction in the rate of tumor cell proliferation, viability, or metastatic activity. A possible way of showing anti-tumor activity is to show a decline in growth rate of abnormal cells that arises during therapy or tumor size stability or reduction. Such activity can be assessed using accepted in vitro or in vivo tumor models, including but not limited to xenograft models, allograft models, MMTV models, and other known models known in the art to investigate anti-tumor activity.

The terms “treat,” “treating,” and “treatment” are meant to include alleviating or abrogating a condition, disorder, or disease, or one or more of the symptoms associated with the condition, disorder, or disease; or alleviating or eradicating the cause(s) of the condition, disorder, or disease itself.

The terms “prevent,” “preventing,” and “prevention” are meant to include a method of delaying and/or precluding the onset of a condition, disorder, or disease, and/or its attendant symptoms; barring a subject from acquiring a condition, disorder, or disease; or reducing a subject’s risk of acquiring a condition, disorder, or disease.

The term “substituted” means that the specified group or moiety bears one or more substituents including, but not limited to, substituents such as alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, alkyl, alkenyl, alkynyl, heterocyclyl, aralkyl, aminosulfonyl, sulfonylamino, sulfonyl, oxo, and the like. The term “unsubstituted” means that the specified group bears no substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

The term “Q-tag,” as used herein, refers to a portion of a polypeptide containing glutamine residue that, upon transglutaminase-mediated reaction with a compound containing -NH₂ amine, provides a conjugate containing the portion of polypeptide, in which the glutamine residue includes a side chain modified to include the amide bonded to the compound. Q-tags are known in the art. Non-limiting examples of Q-tags are LLQGG and GGGLLQGG. In some embodiments, the Q-tag is attached to the C terminal of the heavy chain of the antibody. In some embodiments, the Q-tag is attached to the light chain of the antibody. In some embodiments, the Q-tag is naturally occurring (e.g., the glutamine residue is part of the sequence of the protein). For example, mutation of N297 to N297A exposes Q295 of the antibody, where the conjugation could occur (numbering according to EU index, e.g., as listed in Edelman, G.M. et al., Proc. Natl. Acad. USA, 63, 78-85 (1969) and Kabat, E.A. et al., Sequences of proteins of immunological interest. 5^{th} Edition - US Department of Health and Human Services, NIH publication n° 91-3242, pp 662,680,689 (1991)).. In some embodiments, the Q-tag is within the Fc domain of the antibody.

### II. Conjugates

Targeted delivery of therapeutic agents to diseased or disordered cells remains a topic of great interest for the development of new medical treatments and the supplementation of existing therapeutic modalities. The flexibility of the bioconjugates to provide tailored combinations of targeting biomolecules, such as proteins, peptides, antibodies, or antigen-binding fragments thereof, with specific therapeutics, including but not limited to small molecules and nucleic acids, offers innumerable possibilities for treating a wide array of disorders. Provided in the present disclosure are bioconjugates, and specifically antibody-oligonucleotide conjugates, for targeted treatment of hyperproliferative disorders, immune or inflammatory disorders, and/or infectious diseases.

In one aspect, provided herein is bioconjugate of formula (A):

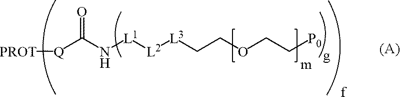

or a stereoisomer, a mixture of two or more diastereomers, a tautomer, or a mixture of two or more tautomers thereof; or a pharmaceutically acceptable salt, solvate, or hydrate thereof; wherein
Q is a glutamine residue, wherein each glutamine residue independently is part of the sequence of PROT or is part of a Q-tag peptide sequence attached to PROT;
PROT is a biomolecule (e.g., peptide, protein, antibody, antigen-binding fragment, etc.) connected to the rest of the conjugate via one or more glutamine residues Q;
each L¹ is independently unsubstituted or substituted alkyl,
each L² is unsubstituted or substituted aryl, or unsubstituted or substituted heteroaryl,
each L³ is independently absent or a linker moiety,
f is an integer selected from the group consisting of 1-20,
each g is independently 0 or 1, provided that at least one g of formula (A) is nonzero,
m is an integer selected from the group consisting of 0-50, and
P₀ is a small molecule (e.g., a small molecule agonist or antagonist), natural product, DNA molecule (e.g., immunomodulating oligonucleotide), RNA molecule (e.g., RNA, siRNA, antisense oligonucleotide, CRISPR guide RNA, etc.), other nucleic acid, CRISPR complex, or carbohydrate.

In some embodiments of the present aspect, P₀ is an immunomodulating polynucleotide or immunomodulating oligonucleotide. In certain embodiments, the immunomodulating polynucleotide or immunomodulating oligonucleotide is an immunostimulating polynucleotide or immunostimulating oligonucleotide.

Immunostimulating polynucleotides have been used in a variety of therapeutic applications. To improve targeting specificity and in vivo distribution, the immunomodulating polynucleotides (e.g., CpG ODNs) can be conjugated to a targeting moiety (e.g., polypeptides). Particularly, transglutaminase-mediated reaction can be used to conduct such a conjugation reaction due to its high reaction rates and suitable site specificity. However, there are still drawbacks remaining with existing transglutaminase-mediated conjugating methods. For example, transglutaminase (Tgase) deamidation leads to the formation of glutamic acid which does not conjugate. To achieve a high degree of conjugation, the ratio of amine to glutamine residue typically needs to be >50:1. Deconjugation occurs even at low temperatures (e.g., 4° C.). Benchmark Q-tag (LLQGG) deconjugates and can be proteolyzed by Tgase giving LLEGG and LLE. Removal of Tgase is inefficient using commercially available methods (e.g., size exclusion chromatography). Herein, the inventors have discovered that oligonucleotide-polypeptide conjugates containing certain linking moieties show improved stability and have favorable properties during preparation using transglutaminase-mediated reactions.

Provided herein is an oligonucleotide-antibody conjugate wherein the oligonucleotide and antibody are attached together via a linking moiety. In some embodiments, one antibody can be conjugated to one or more oligonucleotides. In some embodiments, the oligonucleotide-antibody conjugate is a conjugate of Formula (A1):

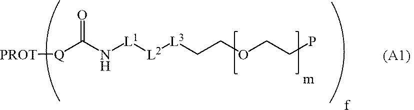

or a stereoisomer, a mixture of two or more diastereomers, a tautomer, or a mixture of two or more tautomers thereof; or a pharmaceutically acceptable salt, solvate, or hydrate thereof; wherein:
Q is a glutamine residue, wherein each glutamine residue independently is part of the sequence of PROT or is part of a Q-tag peptide sequence attached to PROT;
PROT is a protein connected to the rest of the conjugate via one or more glutamine residues Q;
each L¹ is independently unsubstituted or substituted alkyl,
each L² is unsubstituted or substituted aryl, or unsubstituted or substituted heteroaryl,
each L³ is independently absent or a linker moiety,
f is an integer selected from the group consisting of 1-20,
m is an integer selected from the group consisting of 0-50, and
P is an immunomodulating oligonucleotide.

In one embodiment, the oligonucleotide-antibody conjugate has a DAR ranging from about 1 to about of about 20, from about 1 to about 10, from about 1 to about 8, from about 1 to about 4, or from about 1 to about 2. In another embodiment, the oligonucleotide-antibody conjugate has a DAR of about 1, about 2, about 3, about 4, about 5, about 6, about 7, or about 8.

In another aspect, provided herein is a bioconjugate of formula (B):

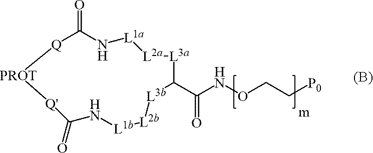

or a stereoisomer, a mixture of two or more diastereomers, a tautomer, or a mixture of two or more tautomers thereof; or a pharmaceutically acceptable salt, solvate, or hydrate thereof; wherein:
Q and Q′ are each a glutamine residue, wherein each glutamine residue independently is part of the sequence of PROT or is part of a Q-tag peptide sequence attached to PROT;
PROT is a biomolecule (e.g., peptide, protein, antibody, antigen-binding fragment, etc.) connected to the rest of the conjugate via Q and Q′;
L^{1a} and L^{1b} are independently unsubstituted or substituted alkyl,
L^{2a} and L^{2b} are independently absent, unsubstituted or substituted alkyl, unsubstituted or substituted aryl, or unsubstituted or substituted heteroaryl,
L^{3a} and L^{3b} are independently absent or a linker moiety,
m is an integer selected from the group consisting of 0-50, and
P₀ is a small molecule (e.g., a small molecule agonist or antagonist), natural product, DNA molecule (e.g., immunomodulating oligonucleotide), RNA molecule (e.g., RNA, siRNA, antisense oligonucleotide, CRISPR guide RNA, etc.), other nucleic acid, CRISPR complex, or carbohydrate.

In some embodiments of the present aspect, P₀ is an immunomodulating polynucleotide or immunomodulating oligonucleotide. In certain embodiments, the immunomodulating polynucleotide or immunomodulating oligonucleotide is an immunostimulating polynucleotide or immunostimulating oligonucleotide.

In some embodiments, one oligonucleotide can be conjugated to an antibody through more than one attachment sites. In some embodiments, the oligonucleotide-antibody conjugate is a conjugate of Formula (B1):

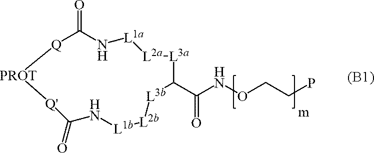

or a stereoisomer, a mixture of two or more diastereomers, a tautomer, or a mixture of two or more tautomers thereof; or a pharmaceutically acceptable salt, solvate, or hydrate thereof; wherein:
Q and Q′ are each a glutamine residue, wherein each glutamine residue independently is part of the sequence of PROT or is part of a Q-tag peptide sequence attached to PROT;
PROT is a protein connected to the rest of the conjugate via Q and Q′;
L^{1a} and L^{1b} are independently unsubstituted or substituted alkyl,
L^{2a} and L^{2b} are independently absent, unsubstituted or substituted alkyl, unsubstituted or substituted aryl, or unsubstituted or substituted heteroaryl,
L^{3a} and L^{3b} are independently absent or a linker moiety,
m is an integer selected from the group consisting of 0-50, and
P is an immunomodulating oligonucleotide.

### Oligonucleotide

In one aspect, the oligonucleotide in the oligonucleotide-antibody conjugate is an immunomodulating (e.g., immunostimulating) polynucleotide. In certain embodiments, the immunomodulating polynucleotide comprises a 5-modified uridine or 5-modified cytidine. In certain embodiments, the inclusion of 5-modified uridine (e.g., 5-ethynyl-uridine) at the 5′-terminus of the immunomodulating polynucleotide (e.g., among the two 5′-terminal nucleosides) enhances the immunomodulating properties of the polynucleotide. In certain embodiments, the immunomodulating polynucleotide is shorter (e.g., comprising a total of from about 6 to about 16 nucleotides or from about 12 to about 14 nucleotides) than a typical CpG ODN, which is from 18 to 28 nucleotides in length. In certain embodiments, the shorter immunomodulating polynucleotide (e.g., those comprising a total of from about 6 to about 16 nucleotides or from about 12 to about 14 nucleotides) retains the immunomodulating activity of a longer, typical CpG ODN; or exhibits higher immunomodulating activity (e.g., as measured by NFκB activation or by the changes in the expression levels of at least one cytokine (e.g., IL-6 or IL-10), as compared to the longer CpG ODN. In certain embodiments, the immunomodulating polynucleotide comprises an abasic spacer. In certain embodiments, the immunomodulating polynucleotide comprises an internucleoside phosphotriester.

In certain embodiments, the immunomodulating polynucleotide provided herein exhibits stability (e.g., stability against nucleases) that is superior to that of a CpG ODN containing mostly internucleoside phosphate (e.g., more than 50% of internucleoside phosphates) without substantially sacrificing its immunostimulating activity. This effect can be achieved, e.g., by incorporating at least 50% (e.g., at least 70%) internucleoside phosphorothioates or phosphorodithioates or through the inclusion of internucleoside phosphotriesters and/or internucleoside abasic spacers. Phosphotriesters and abasic spacers are also convenient for conjugation to a targeting moiety. Phosphate-based phosphotriesters and abasic spacers can also be used for reduction of off-target activity, relative to polynucleotides with fully phosphorothioate backbones. Without wishing to be bound by theory, this effect may be achieved by reducing self-delivery without disrupting targeting moiety-mediated delivery to target cells. Accordingly, a polynucleotide provided herein can include about 15 or fewer, about 14 or fewer, about 13 or fewer, about 12 or fewer, about 11 or fewer, or about 10 or fewer contiguous internucleoside phosphorothioates. For example, an immunostimulating polynucleotide comprising a total of from about 12 to about 16 nucleosides can contain about 10 or fewer contiguous internucleoside phosphorothioates.

The immunostimulating polynucleotide provided herein can contain a total of about 50 or fewer, about 30 or fewer, about 28 or fewer, or about 16 or fewer nucleosides. The immunostimulating polynucleotide can contain a total of at least 6, about 10 or more, or about 12 or more nucleosides. For example, the immunostimulating polynucleotide can contain a total of from about 6 to about 30, from about 6 to about 28, from about 6 to about 20, from about 6 to about 16, from about 10 to about 20, from about 10 to about 16, from about 12 to about 28, from about 12 to about 20, or from about 12 to about 16 nucleosides.

In certain embodiments, the immunostimulating polynucleotide comprises one or more phosphotriesters (e.g., internucleoside phosphotriesters) and/or phosphorothioates (e.g., from about 1 to about 6 or from about 1 to about 4), e.g., at one or both termini (e.g., within the six 5′-terminal nucleosides or the six 3′-terminal nucleosides). The inclusion of one or more internucleoside phosphotriesters and/or phosphorothioates can enhance the stability of the polynucleotide by reducing the rate of exonuclease-mediated degradation.

In certain embodiments, the immunostimulating polynucleotide comprises a phosphotriester or a terminal phosphodiester, where the phosphotriester or the terminal phosphodiester comprises a linker bonded to a targeting moiety or a conjugating group and optionally to one or more (e.g., from about 1 to about 6) auxiliary moieties. In certain embodiments, the immunostimulating polynucleotide comprises only one linker. In certain embodiments, the immunostimulating polynucleotide comprises only one conjugating group.

The polynucleotide provided herein can be a hybridized polynucleotide including a strand and its partial or whole complement. The hybridized polynucleotides can have at least 6 complementary base pairings (e.g., about 6, about 7, about 8, about 9, about 10, about 11, about 12, about 13, about 14, about 15, about 16, about 17, about 18, about 19, about 20, about 21, about 22, or about 23), up to the total number of the nucleotides present in the included shorter strand. For example, the hybridized portion of the hybridized polynucleotide can contain about 6, about 7, about 8, about 9, about 10, about 11, about 12, about 13, about 14, about 15, about 16, about 17, about 18, about 19, about 20, about 21, about 22, or about 23 base pairs.

In one aspect, the oligonucleotide in the oligonucleotide-antibody conjugate comprises one or more CpG sites. In some embodiments, the oligonucleotide comprises at least 1, at least 2, or at least 3 CpG sites. In some embodiments, the oligonucleotide is an antisense oligonucleotide As used herein, a “modified nucleotide” is a nucleotide other than a ribonucleotide (2′-hydroxyl nucleotide). In some embodiments, at least 50%, at least 55%, at least 60%, at least 65%, at least 70%, at least 75%, at least 80%, at least 85%, at least 90%, at least 95%, at least 97%, at least 98%, at least 99%, or 100% of the nucleotides are modified nucleotides. As used herein, modified nucleotides include, but are not limited to, deoxyribonucleotides, nucleotide mimics, abasic nucleotides, 2′-modified nucleotides, 3′ to 3′ linkages (inverted) nucleotides, non-natural base-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides (cPrpN). The 2′-modified nucleotides (i.e. a nucleotide with a group other than a hydroxyl group at the 2′ position of the five-membered sugar ring) include, but are not limited to, 2′-O-alkyl nucleotides, 2′-deoxy-2′-halo nucleotides, 2′-deoxy nucleotides, 2′-methoxyethyl (2′-O-2-methoxylethyl) nucleotides, 2′-amino nucleotides, 2′-aminoalkyl nucleotides, and 2′-alkyl nucleotides. In some embodiments, modified nucleotide is selected from the group consisting of 5-bromo-2′-O-methyluridine, 5-bromo-2′-deoxyuridine, 2′-O-methylthymidine, 2′-O-methylcytidine, 2′-O-(2-methoxyethyl)thymidine and 8-oxo-7,8-dihydro-2′-deoxyguanosine. It is not necessary for all positions in a given compound to be uniformly modified. Conversely, more than one modification may be incorporated in a single oligonucleotide or even in a single nucleotide thereof. The oligonucleotides may be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-Me-C), 5-hydroxymethyl cytosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil (e.g., 5-bromouracil and 5-iodouracil), cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl, 8-oxo and other 8-substituted adenines and guanines, 5-halo (e.g., 5-bromo and 5-iodo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, one or more nucleotides of the oligonucleotide are linked by non-standard linkages or backbones (e.g., modified internucleoside linkages or modified backbones). In some embodiments, a modified internucleoside linkage is a non-phosphate-containing covalent internucleoside linkage. Modified internucleoside linkages or backbones include, but are not limited to, 5′-phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, the oligonucleotide comprises at least 2, at least 3, at least 4, at least 5, at least 6, at least 7, at least 8, at least 9, at least 10, at least 11, at least 12, at least 13, at least 14, or at least 15 phosphorothioate linkages. In some embodiments, the oligonucleotide comprises at least 2, at least 3, at least 4, at least 5, at least 6, at least 7, at least 8, at least 9, at least 10, at least 11, at least 12, at least 13, at least 14, or at least 15 phosphorodithioate linkages. In some embodiments, the oligonucleotide comprises 1, 2, 3, 4, 5, 6, 7, 8, 9, 10, 11, 12, 13, 14, or 15 phosphorothioate linkages. In some embodiments, the oligonucleotide comprises 1, 2, 3, 4, 5, 6, 7, 8, 9, or 10 phosphorodithioate linkages. In some embodiments, the phosphorothioate internucleoside linkages or phosphorodithioate internucleoside linkages are between the nucleotides at positions 1-3, 2-4, 3-5, 4-6, 4-5, 6-8, 7-9, 8-10, 9-11, 10-12, 11-13, 12-14, 13-15, 14-16, 15-17, 16-18, 17-19, 18-20 or 19-21 from the 5′ end of the oligonucleotide. In some embodiments, the oligonucleotide comprises one or more modified nucleotides and one or more modified internucleoside linkages.

In some embodiments, the oligonucleotide comprises a terminal cap. In some embodiments, the terminal cap is at the 3′ end of the oligonucleotide. In some embodiments, the terminal cap is at the 5′ end of the oligonucleotide. In some embodiments, the terminal cap is at the 5′ end and 3′ end of the oligonucleotide. The term “terminal cap” can also be referred to as “cap,” and has meaning generally accepted in the art. For example, the term refers to a moiety, which can be a chemically modified nucleotide or non-nucleotide that can be incorporated at one or more termini of one or more nucleic acid molecules of the invention. These terminal modifications can protect the nucleic acid molecule from exonuclease degradation, and can help in delivery and/or localization within a cell. In non-limiting examples, the cap includes, but is not limited to a polymer; a ligand; locked nucleic acid (LNA); glyceryl; an abasic ribose residue; inverted deoxy abasic residue; an inverted nucleotide; 4′,5′-methylene nucleotide; 1-(beta-D-erythrofuranosyl) nucleotide; 5′-mercapto moieties; 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide; 3′-3′-inverted nucleotide moiety; 3′ -3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; 5′-5′-inverted nucleotide moiety; 5′-5′-inverted abasic moiety; 5′-phosphoramidate; 5′-phosphorothioate; 1,4-butanediol phosphate; 5′-amino; bridging and/or nonbridging 5′-phosphoramidate; phosphorothioate and/or phosphorodithioate; or bridging or non-bridging methylphosphonate moiety. In some embodiments, the oligonucleotide comprises one or more terminal cap molecules. In some embodiments, [N] is a 3′ terminal cap. In some embodiments, the 3′ terminal cap is O-(3-hydroxypropyl)phosphorothioate.

In some embodiments, the oligonucleotide is about 10-30, about 10-15, about 15-20, about 20-25, about 25-30, about 15-25 nucleotides in length. In some embodiments, the oligonucleotide is about 18, 19, 20, 21, 22, 23, 24 or 25 nucleotides in length.

In another aspect, the oligonucleotide of the oligonucleotide-antibody conjugate is:

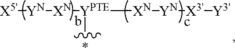

wherein
b and c are each independently an integer from 1 to 25; with the proviso that the sum of b and c is at least 5;

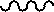

* indicates the point of attachment of the immunomodulating oligonucleotide P to the rest of the conjugate;

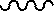

X^{5′} is a 5′ terminal nucleoside having the structure

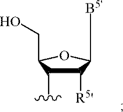

X^{3′} is a 3′ terminal nucleoside having the structure

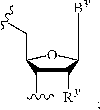

Y^{PTE} is an internucleoside phosphotriester having the structure

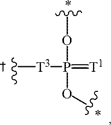

wherein * indicates the points of attachment to the rest of the oligonucleotide and † indicates the point of attachment to the rest of the conjugate;
Y^{3′} is a terminal phosphotriester having the structure

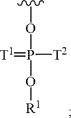

each X^{N} is independently a nucleoside having the structure

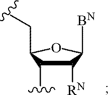

each Y^{N} is independently an internucleoside linker having the structure

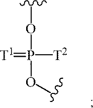

wherein each B^{N} is independently a modified or unmodified nucleobase;
each R^{N} is independently —H or —O—C₁₋₄-alkyl, wherein the C₁₋₄-alkyl of the —O—C₁₋₄-alkyl is further optionally substituted by —O—C₁-C₄-alkyl;
B^{5′} and B^{3′} are independently a modified or unmodified nucleobase;
R^{5′} and R^{3′} are independently —H or —O—C₁-C₄-alkyl, wherein the C₁₋₄-alkyl of the —O—C₁₋₄-alkyl is further optionally substituted by —O—C₁-C₄-alkyl;
each T₁ is independently O or S;
each T₂ is independently O⁻ or S⁻; and
T₃ is a group comprising an oligoethylene glycol moiety; and
R¹ is C₁₋₄-alkylene-hydroxy.

In certain embodiments, the oligonucleotide comprises a nucleotide with a modified nucleobase.

In certain embodiments, b is an integer ranging from about 1 to about 15. In certain embodiments, b is an integer of about 1, about 2, about 3, about 4, about 5, about 6, about 7, about 8, about 9, about 10, about 11, about 12, about 13, about 14, or about 15. In certain embodiments, b is an integer of about 3, about 4, about 11, or about 14. In certain embodiments, b is an integer of about 3. In certain embodiments, b is an integer of about 4. In certain embodiments, b is an integer of about 11. In certain embodiments, b is an integer of about 14.

In certain embodiments, c is an integer ranging from about 0 to about 10. In certain embodiments, c is an integer of about 0, about 1, about 2, about 3, about 4, about 5, about 6, about 7, about 8, about 9, or about 10. In certain embodiments, c is an integer of about 0 or about 8. In certain embodiments, c is an integer of about 0. In certain embodiments, c is an integer of about 8.

In certain embodiments, b is an integer of about 3 and c is an integer of about 8. In certain embodiments, b is an integer of about 4 and c is an integer of about 8. In certain embodiments, b is an integer of about 11 and c is an integer of about 0. In certain embodiments, b is an integer of about 14 and c is an integer of about 0.

In certain embodiments, b and c together in total are ranging from about 5 to about 20. In certain embodiments, b and c together in total are ranging from about 5 to about 15. In certain embodiments, b and c together in total are about 5, about 6, about 7, about 8, about 9, about 10, about 11, about 12, about 13, about 14, or about 15. In certain embodiments, b and c together in total are about 8, about 9, about 10, about 11, about 12, about 13, or about 14. In certain embodiments, b and c together in total are about 11. In certain embodiments, b and c together in total are about 12. In certain embodiments, b and c together in total are about 14.

In certain embodiments, each X^{N} is independently a 2′-deoxyribonucleoside or a 2′-modified ribonucleoside. In certain embodiments, each X^{N} is independently 2′-deoxyadenosine (A), 2′-deoxyguanosine (G), 2′-deoxycytidine (C), a 5-halo-2′-deoxycytidine, 2′-deoxythymidine (T), 2′-deoxyuridine (U), a 5-halo-2′-deoxyuridine, a 2′-fluororibonucleoside, a 2′-methoxyribonucleoside, or a 2′-(2-methoxyethoxy)ribonucleoside. In certain embodiments, each X^{N} is independently a 2′-deoxyribonucleoside. In certain embodiments, each X^{N} is independently 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, each X^{N} is independently 2′-deoxyadenosine, 2′-deoxyguanosine, 2′-deoxycytidine, 2′-deoxythymidine, 5-bromo-2′-deoxyuridine, or 5-iodo-2′-deoxyuridine.

In certain embodiments, X^{3′} is a 2′-deoxyribonucleoside or a 2′-modified ribonucleoside. In certain embodiments, X^{3′} is a 2′-deoxyribonucleoside. In certain embodiments, X^{3′} is 2′-deoxyadenosine, 2′-deoxyguanosine, 2′-deoxycytidine, a 5-halo-2′-deoxycytidine, 2′-deoxythymidine, 2′-deoxyuridine, a 5-halo-2′-deoxyuridine, a 2′-fluororibonucleoside, a 2′-methoxyribonucleoside, or a 2′-(2-methoxyethoxy)ribonucleoside. In certain embodiments, X^{3′} is 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, X^{3′} is 2′-deoxythymidine. In certain embodiments, X^{3′} is a 2′-deoxyribonucleoside with a substituted pyrimidine base. In certain embodiments, X^{3′} is a 2′-deoxyribonucleoside with a 5-substituted pyrimidine base. In certain embodiments, X^{3′} is 2′-deoxythymidine, a 5-halo-2′-deoxycytidine, or a 5-halo-2′-deoxyuridine. In certain embodiments, X^{3′} is 2′-deoxythymidine, 5-bromo-2′-deoxycytidine, 5-iodo-2′-deoxycytidine, 5-bromo-2′-deoxyuridine, or 5-iodo-2′-deoxyuridine. In certain embodiments, X^{3′} is 2′-deoxythymidine, 5-bromo-2′-deoxyuridine, or 5-iodo-2′-deoxyuridine. In certain embodiments, X^{3′} is a terminal nucleoside comprising a 3′ capping group. In certain embodiments, the 3′ capping group is a terminal phosphoester. In certain embodiments, the 3′ capping group is 3-hydroxyl-propylphosphoryl (i.e., —P(O₂)—OCH₂CH₂CH₂OH).

In certain embodiments, X^{5′} is a 2′-deoxyribonucleoside or a 2′-modified ribonucleoside. In certain embodiments, X^{5′} is a 2′-deoxyribonucleoside. In certain embodiments, X^{5′} is 2′-deoxyadenosine, 2′-deoxyguanosine, 2′-deoxycytidine, a 5-halo-2′-deoxycytidine, 2′-deoxythymidine, 2′-deoxyuridine, a 5-halo-2′-deoxyuridine, a 2′-fluororibonucleoside, a 2′-methoxyribonucleoside, or a 2′-(2-methoxyethoxy)ribonucleoside. In certain embodiments, X^{5′} is 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, X^{5′} is a 2′-deoxyribonucleoside with a substituted pyrimidine base. In certain embodiments, X^{5′} is a 2′-deoxyribonucleoside with a 5-substituted pyrimidine base. In certain embodiments, X^{5′} is 2′-deoxythymidine, a 5-halo-2′-deoxycytidine, or a 5-halo-2′-deoxyuridine. In certain embodiments, X^{5′} is a 5-halo-2′-deoxycytidine. In certain embodiments, X^{5′} is a 5-halo-2′-deoxyuridine. In certain embodiments, X^{5′} is 2′-deoxythymidine, 5-bromo-2′-deoxycytidine, 5-iodo-2′-deoxycytidine, 5-bromo-2′-deoxyuridine, or 5-iodo-2′-deoxyuridine. In certain embodiments, X^{5′} is 2′-deoxythymidine, 5-bromo-2′-deoxyuridine, or 5-iodo-2′-deoxyuridine. In certain embodiments, X^{5′} is 5-bromo-2′-deoxyuridine. In certain embodiments, X^{5′} is 5-iodo-2′-deoxyuridine. In certain embodiments, X^{5′} has a 3′-phosphorothioate group. In certain embodiments, X^{5′} has a 3′-phosphorothioate group with a chirality of Rp. In certain embodiments, X^{5′} has a 3′-phosphorothioate group with a chirality of Sp.

In certain embodiments, Y^{PTE} is an internucleoside phosphothiotriester.

In some embodiments, Y^{PTE} is

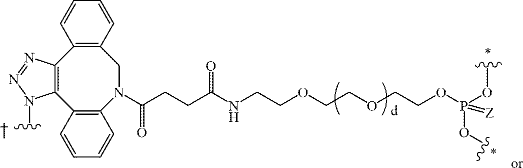

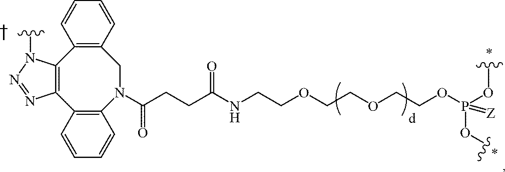

wherein Z is O or S; d is an integer from 0 to 95; the two

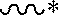 [SMILES: *C]

on the right side of the structure indicate the points of attachment to the adjacent nucleosides X^{N} in the oligonucleotide P, and the one † on the left side of the structure indicates the point of attachment to the rest of the conjugate. In certain embodiments, Z is O. In certain embodiments, Z is S. In certain embodiments, d is an integer ranging from about 0 to about 10. In certain embodiments, d is an integer ranging from about 0 to about 5. In certain embodiments, d is an integer of about 0, about 1, about 2, about 3, about 4, or about 5. In certain embodiments, d is an integer of about 0, about 1, or about 3.

In some embodiments, Y^{PTE} is

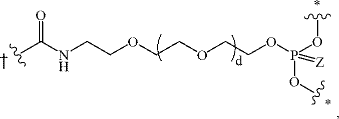

wherein Z is O or S; d is an integer from 0 to 95; the two

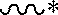 [SMILES: *C]

on the right side of the structure indicate the points of attachment to the adjacent nucleosides X^{N} in the oligonucleotide P, and the † on the left side of the structure indicates the point of attachment to the rest of the conjugate. In certain embodiments, Z is O. In certain embodiments, Z is S. In certain embodiments, d is an integer ranging from about 0 to about 10. In certain embodiments, d is an integer ranging from about 0 to about 5. In certain embodiments, d is an integer of about 0, about 1, about 2, about 3, about 4, or about 5. In certain embodiments, d is an integer of about 0, about 1, or about 3.

In certain embodiments, the oligonucleotide comprises one additional internucleoside phosphotriester. In one embodiment, the additional internucleoside phosphotriester is a C₁₋₆ alkylphosphotriester. In another embodiment, the additional internucleoside phosphotriester is ethylphosphotriester.

In certain embodiments, the oligonucleotide comprises one 5-halo-2′-deoxyuridine. In one embodiment, the 5-halo-2′-deoxyuridine is 5-fluoro-2′-deoxyuridine, 5-bromo-2′-deoxyuridine, or 5-iodo-2′-deoxyuridine. In another embodiment, the 5-halo-2′-deoxyuridine is 5-bromo-2′-deoxyuridine or 5-iodo-2′-deoxyuridine. In yet another embodiment, the 5-halo-2′-deoxyuridine is 5-fluoro-2′-deoxyuridine. In yet another embodiment, the 5-halo-2′-deoxyuridine is 5-bromo-2′-deoxyuridine. In still another embodiment, the 5-halo-2′-deoxyuridine is 5-iodo-2′-deoxyuridine.

In certain embodiments, the oligonucleotide comprises three or more 2′-deoxycytidines. In certain embodiments, the oligonucleotide comprises three 2′-deoxycytidines.

In certain embodiments, the oligonucleotide comprises four or more 2′-deoxyguanosines. In certain embodiments, the oligonucleotide comprises four 2′-deoxyguanosines.

In certain embodiments, the oligonucleotide comprises three 2′-deoxycytidines and four 2′-deoxyguanosines. In certain embodiments, the oligonucleotide comprises one, two, or three CG dinucleotides. In certain embodiments, the oligonucleotide comprises three CG dinucleotides.

In certain embodiments, the oligonucleotide comprises three or more 2′-deoxythymidines. In certain embodiments, the oligonucleotide comprises three, four, five, six, seven, or eight 2′-deoxythymidines. In certain embodiments, the oligonucleotide comprises three, four, five, or eight 2′-deoxythymidines.

In certain embodiments, the oligonucleotide does not comprise a 2′-deoxyadenosine. In certain embodiments, the oligonucleotide comprises one or two 2′-deoxyadenosines.

In certain embodiments, the oligonucleotide has a length ranging from about 5 to about 20 or from about 6 to about 15. In certain embodiments, the oligonucleotide has a length of about 6, about 7, about 8, about 9, about 10, about 11, about 12, about 13, about 14, or about 15. In certain embodiments, the oligonucleotide has a length of about 10, about 11, about 12, about 13, about 14, or about 15.

In certain embodiments, the oligonucleotide comprises one or more internucleoside phosphorothioates. In certain embodiments, all the internucleoside phosphoesters in the oligonucleotide are internucleoside phosphorothioates. In certain embodiments, the oligonucleotide comprises one or more chiral internucleoside phosphorothioates.

In certain embodiments, the oligonucleotides comprising a sequence of N₁N₂CGN₃CG(T)ₓGN₄CGN₅T, or a stereoisomer, a mixture of two or more diastereomers, a tautomer, or a mixture of two or more tautomers thereof; or a pharmaceutically acceptable salt, solvate, or hydrate thereof are as described in, for example, WO2018/189382 A1.

In one embodiment, the oligonucleotide comprises a sequence of N¹N²CGN³CG(T)ₓGN⁴CGN⁵T, or a stereoisomer, a mixture of two or more diastereomers, a tautomer, or a mixture of two or more tautomers thereof; or a pharmaceutically acceptable salt, solvate, or hydrate thereof; wherein:
x is an integer ranging from about 1 to about 4;
N¹ is absent or 2′-deoxythymidine;
N² is a 2′-deoxyribonucleotide with a modified nucleobase;
N³ is 2′-deoxyadenosine or 2′-deoxythymidine, each optionally comprising a 3′-phosphotriester;
N⁴ is 2′-deoxyadenosine or 2′-deoxythymidine;
N⁵ is 2′-deoxythymidine optionally comprising a 3′-phosphotriester; and
C is 2′-deoxycytidine and G is 2′-deoxyguanosine.

In certain embodiments, in N¹N²CGN³CG(T)ₓGN⁴CGN⁵T, x is an integer of about 1, about 2, about 3, or about 4. In certain embodiments, in N¹N²CGN³CG(T)ₓGN⁴CGN⁵T, x is an integer of about 1. In certain embodiments, in N¹N²CGN³CG(T)ₓGN⁴CGN⁵T, x is an integer of about 4.

In certain embodiments, in N¹N²CGN³CG(T)ₓGN⁴CGN⁵T, N¹ is absent. In certain embodiments, in N¹N²CGN³CG(T)ₓGN⁴CGN⁵T, N¹ is 2′-deoxythymidine.

In certain embodiments, in N¹N²CGN³CG(T)ₓGN⁴CGN⁵T, N² is a 2′-deoxyribonucleotide with a substituted pyrimidine base. In certain embodiments, in N¹N²CGN³CG(T)ₓGN⁴CGN⁵T, N² is a 2′-deoxyribonucleotide with a 5-substituted pyrimidine base. In certain embodiments, in N¹N²CGN³CG(T)ₓGN⁴CGN⁵T, N² is a 5-halo-2′ -deoxycytidine or a 5-halo-2′-deoxyuridine. In certain embodiments, in N′N 2CGN³ CG(T)ₓGN⁴CGN⁵T, N² is 5-bromo-2′-deoxyuridine or 5-iodo-2′-deoxyuridine.

In certain embodiments, in N¹N²CGN³CG(T)ₓGN⁴CGN⁵T, N³ is 2′-deoxyadenosine comprising a 3′-phosphotriester. In certain embodiments, in N¹N²CGN³CG(T)ₓGN⁴CGN⁵T, N³ is 2′-deoxythymidine. In certain embodiments, in N¹N²CGN³CG(T)ₓGN⁴CGN⁵T, N³ is 2′-deoxythymidine comprising a 3′-phosphotriester.

In certain embodiments, in N¹N²CGN³CG(T)ₓGN⁴CGN⁵T, N⁴ is 2′-deoxyadenosine. In certain embodiments, in N¹N²CGN³CG(T)ₓGN⁴CGN⁵T, N⁴ is 2′-deoxythymidine.

In certain embodiments, in N¹N²CGN³CG(T)ₓGN⁴CGN⁵T, N⁵ is 2′-deoxythymidine. In certain embodiments, in N¹N²CGN³CG(T)ₓGN⁴CGN⁵T, N⁵ is 2′-deoxythymidine comprising a 3′-phosphotriester.

In certain embodiments, the oligonucleotide of N¹N²CGN³CG(T)ₓGN⁴CGN⁵T comprises one or more internucleoside phosphorothioates or phosphorotdithioates. In certain embodiments, the oligonucleotide of N¹N²CGN³CG(T)ₓGN⁴CGN⁵T comprises at least one chiral internucleoside phosphorothioate or phosphorotdithioates. In certain embodiments, the oligonucleotide of N¹N²CGN³CG(T)ₓGN⁴CGN⁵T comprises at least one chiral phosphorotdithioates. In certain embodiments, the oligonucleotide of N¹N²CGN³CG(T)ₓGN⁴CGN⁵T is an oligonucleotide sequence as described in, for example, WO2018/189382 A1.

In certain embodiments, the oligonucleotide provided herein is an immunostimulating polynucleotide. In certain embodiments, the oligonucleotide provided herein functions as a PAMS. In certain embodiments, the oligonucleotide provided herein activates innate immune response or stimulates the adaptive immune response by triggering TLR9 signaling. In certain embodiments, the oligonucleotide provided herein is a TLR9 agonist.

In certain embodiments, the oligonucleotide provided herein is a CpG oligonucleotide, comprising a modification including 5-halouridine or 5-alkynyluridine, or a truncated version thereof (e.g., those comprising a total of about 6 to about 16 nucleosides). In certain embodiments, the truncated oligonucleotide provided herein comprises a truncated oligonucleotide sequence, from which one or more 3′-terminal nucleotides are eliminated or one or more of the intra-sequence nucleotides excised.

In certain embodiments, the oligonucleotide provided herein comprises at least one immunostimulating sequence (ISS). In certain embodiments, the oligonucleotide provided herein comprises about 1, about 2, about 3, or about 4 ISS. The ISS in immunostimulating polynucleotides is dependent on the targeted organism. The common feature of the ISS used in the oligonucleotide provided herein is the cytidine-p-guanosine sequence, in which p is an internucleoside phosphodiester (e.g., phosphate or phosphorothioate) or an internucleoside phosphotriester. In certain embodiments, cytidine and guanosine in the ISS each independently comprises 2′-deoxyribose. In certain embodiments, the oligonucleotide provided herein comprises about 1, about 2, or about 3 human ISSs. In certain embodiments, the human ISS is CG or NCG, where N is uridine, cytidine, or thymidine, or a modified uridine or cytidine; and G is guanosine or a modified guanosine. In certain embodiments, the modified uridine or cytidine is a 5-halouridine (e.g., 5-iodouridine or 5-bromouridine), a 5-alkynyluridine (e.g., 5-ethynyluridine or 5-propynyluridine), 5-heteroaryluridine, or 5-halocytidine. In certain embodiments, the modified guanosine is 7-deazaguanosine. In certain embodiments, the human ISS is NCG, in one embodiment, N is 5-halouridine. In certain embodiments, the human ISS is UCG, in one embodiment, U is 5-alkynyluridine, and in another embodiment, U is 5-ethynyluridine. In certain embodiments, the oligonucleotide provided herein targeting humans comprises an ISS within four contiguous nucleotides that include a 5′-terminal nucleotide. In certain embodiments, the oligonucleotide provided herein targeting humans comprises a 5′-terminal ISS. In certain embodiments, the oligonucleotide provided herein comprises a murine ISS. In certain embodiments, the murine ISS is a hexameric nucleotide sequence: Pu-Pu-CG-Py-Py, where each Pu is independently a purine nucleotide, and each Py is independently a pyrimidine nucleotide.

In certain embodiments, the 5′-flanking nucleotides relative to CpG in the oligonucleotide provided herein does not contain 2′-alkoxyriboses. In certain embodiments, the 5′-flanking nucleotides relative to CpG in the oligonucleotide provided herein comprises only 2′-deoxyriboses as sugars.

In certain embodiments, the oligonucleotide provided herein has (1) a high content of phosphorothioates or phosphorodithioates (e.g., at least 50%, at least 60%, at least 70%, or at least 80% of nucleosides may be linked by phosphorothioates or phosphorodithioates); (2) absence of poly-G tails; (3) nucleosides in the oligonucleotide comprises 2′-deoxyriboses or 2′-modified riboses (e.g., 2′-halo (e.g., 2′-fluoro, 2′-bromo, or 2′-iodo) or optionally substituted 2′-alkoxy (e.g., 2′-methoxy)); and/or (4) the inclusion of 5′-terminal ISS that is NCG, in which N is uridine, cytidine, or thymidine, or a modified uridine or cytidine, and G is guanosine or a modified guanosine.

In certain embodiments, the oligonucleotide provided herein suppresses the adaptive immune response by reducing activation of TLR9 signaling (e.g., through TLR9 antagonism). In certain embodiments, the immunosuppressive polynucleotide provided herein comprises at least two 2′-alkoxynucleotides that are 5′-flanking relative to CpG as described by the formula of N¹-N²-CG, where N¹ and N² are each independently a nucleotide containing 2′-alkoxyribose (e.g., 2′-methoxyribose).

In some embodiments, the oligonucleotide comprises one or more of unmodified sequences differing by 0, 1, 2 or 3 nucleobases from the sequences shown in Table 1. In some embodiments,, the oligonucleotide comprises one or more of modified sequences differing by 0, 1, 2 or 3 nucleobases from the sequences shown in Table 2.

TABLE 1

Unmodified Oligonucleotides

| SEQ ID NO. | Unmodified Oligonucleotide Sequence (5′→3′) |
|---|---|
| 1 | tucgtcgtgacgtt |
| 2 | ucgtcgtgtcgtt |

TABLE 2

Modified Oligonucleotides *

| SEQ ID NO. | Modified Oligonucleotide Sequence (5′→3′) |
|---|---|
| 3 | uscsgstscsgstsgstscsgstst-c3 |
| 4 | uscsgstscsgstsgstscsgstst-c3 |
| 5 | tsuscsgstscsgstsgsascsgstst-c3 |

^{∗}Modifications
u: 5-Bromo-2′-deoxyuridine
u: 5-iodo-2′deoxyuridine
Bold letter: 2′-OMOE nucleotide
Italic letter: phosphotriester linker-PEG₂₄-NH₂ 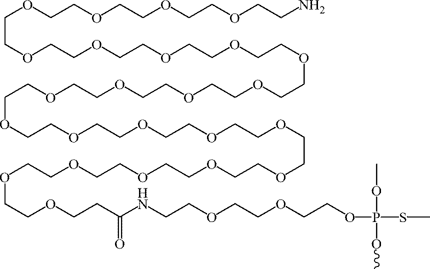
Lower case: 2′-deoxy nucleotide
s: phosphorothioate linkage

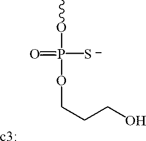

In some embodiments, the oligonucleotide is functionalized with a chemical tag for attachment to the linking moiety. In some embodiments, the chemical tag is attached to an inter-nucleoside linkage of the oligonucleotide. In some embodiments, the chemical tag is attached to a 5′ inter-nucleoside linkage. In some embodiments, the chemical tag is attached to a 3′ inter-nucleoside linkage. In some embodiments, the inter-nucleoside linkage is a phosphorothioate linkage. In some embodiments, the inter-nucleoside linkage is a phosphorodithioate linkage. In some embodiments, the chemical tag is closer to the 5′ end than the 3′ end of the oligonucleotide. In some embodiments, the chemical tag is attached to a nucleobase.

### Linking Moieties

In another aspect, the oligonucleotide is conjugated to the polypeptide via a linking moiety. The length, rigidity and chemical composition of the linking moiety impact the conjugation reaction rates and the stability of the resulting conjugates. In some embodiments, the linking moiety comprises polyethylene glycol (PEG). In some embodiments, the PEG contains about 10-50 ethylene glycol units. In some embodiments, the linking moiety further comprises an aromatic moiety. In some embodiments, the linking moiety is an aliphatic chain.

For Formula (A) or (A1), the linking moiety is represented by

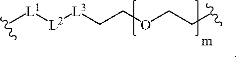

In some embodiments, L¹ is absent. In some embodiments, L¹ is unsubstituted alkyl. In some embodiments, L¹ is independently an unsubstituted C₁₋₆ alkyl. In some embodiments, each L¹ is methyl or ethyl. In some embodiments, L¹ is independently a substituted alkyl. In some embodiments, L¹ is independently a substituted C₁₋₆ alkyl. In some embodiments, L¹ is C₁₋₆ alkyl substituted with one or more substituents selected from the group consisting of alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, alkyl, alkenyl, alkynyl, heterocyclyl, aralkyl, aminosulfonyl, sulfonylamino, sulfonyl and oxo. In some embodiments, L¹ is independently a C₁₋₆ alkyl substituted with one or more aryl. In some embodiments, L¹ is independently a C₁₋₆ alkyl substituted with one aryl. In some embodiments, L¹ is independently a C₁₋₆ alkyl substituted with phenyl.

In some embodiments, L² is independently a 6-10 membered aryl. In some embodiments, each L² is phenyl. In some embodiments, each L² is independently a 6-10 membered heteroaryl. In some embodiments, each L² is pyridinyl. In other embodiments, L² is -NH-(6-10 membered arylene)-, -NH-(6-10 membered heteroarylene)-, -(6-10 membered arylene)-NH-, -(6-10 membered heteroarylene)-NH-, -O-(6-10 membered arylene)-, -O-(6-10membered heteroarylene)-, -(6-10 membered arylene)-O-, or -(6-10 membered heteroarylene)-O-.

In some embodiments, L³ is absent. In some embodiments, L³ is a linker moiety. In some embodiments, the linker moiety is an unsubstituted or substituted alkyl. In some embodiments, the linker moiety is independently an unsubstituted C₁₋₆ alkyl. In some embodiments, the linker moiety is methyl or ethyl. In some embodiments, the linker moiety is independently a substituted alkyl. In some embodiments, the linker moiety is independently a substituted C₁₋₆ alkyl. In other emodiments, the linker moiety is C₁₋₆ alkylene-O-. In some embodiments, the C₁₋₆ alkyl of the linker moiety substituted with one or more substituents selected from the group consisting of alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, alkyl, alkenyl, alkynyl, heterocyclyl, aralkyl, aminosulfonyl, sulfonylamino, sulfonyl and oxo. In some embodiments, the C₁₋₆ alkyl of the linker moiety is substituted with one or more aryl. In some embodiments, the linker moiety is independently a C₁₋₆ alkyl substituted with one aryl. In some embodiments, the linker moiety is independently a C₁₋₆ alkyl substituted with phenyl. In some embodiments, the linker moiety is an amino acid residue. In some embodiments, the amino acid is selected from the group consisting of glycine, alanine, glutamic acid and proline. In some embodiments, the linker is methyl. In some embodiments, the linker moiety is —R⁵C(O)R⁶NHR⁷—, wherein R⁵ and R⁷ are independently absent or unsubstituted or substituted alkyl and R⁶ is an amino acid residue. In some embodiments, the amino acid is selected from the group consisting of glycine, alanine, glutamic acid and proline. In some embodiments, the linker moiety is —R³C(O)NHR⁴— or R³NHC(O)R⁴—, wherein R³ and R⁴ are independently absent or unsubstituted or substituted alkyl. In some embodiments, R³ is methylene and R⁴ is —(CH₂)₄—. In some embodiments, R³ is methylene and R⁴ is absent. When more than one oligonucleotide (i.e., p = 2), the two L¹ can be different or same, the two L² can be different or same and the two L³ can be different or same.

In some embodiments, m is about 0-100, about 0-95, about 0-50, about 0-30, about 0-25, about 1-95, about 1-50, about 50-100, about 3-10, about 10-15, about 15-20, about 20-25, about 25-30, about 5-16, about 15-30, about 15-25 or about 20-30. In some embodiments, m is 20, 21, 22, 23, 24 or 25.

In some embodiments, -L¹-L²-L³- of Formula (A) or (A1) is selected from the group consisting of:

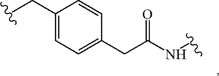

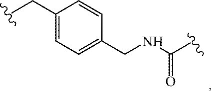

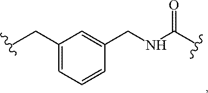

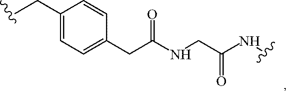

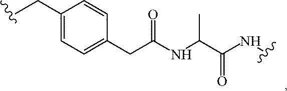

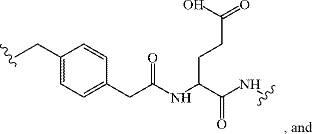

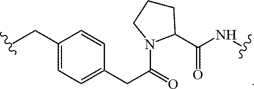

For Formula (B) or (B1), the linking moiety is represented by

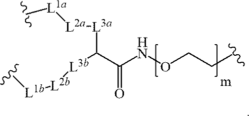

In some embodiments, one or both of L^{1a} and L^{1b} are methyl or ethyl. In some embodiments, one or both of L^{1a} and L^{1b} are substituted by an unsubstituted or substituted aryl. In some embodiments, one or both of L^{1a} and L^{1b} are substituted by phenyl. In some embodiments, one or both of L^{2a} and L^{2b} are absent. In some embodiments, one or both of L^{2a} and L^{2b} are a 6-10 membered aryl. In some embodiments, one or both of L^{2a} and L^{2b} is phenyl. In some embodiments, one or both of L^{2a} and L^{2b} is independently a 6-10 membered heteroaryl. In some embodiments, one or both of L^{2a} and L^{2b} is pyridinyl. In some embodiments, one or both of L^{3a} and L^{3b} are linker moieties. In some embodiments, the linker moiety is an unsubstituted or substituted alkyl. In some embodiments, the linker is methyl. In some embodiments, the linker moiety is —R⁵C(O)R⁶NHR⁷—, wherein R⁵, and R⁷ are independently absent or unsubstituted or substituted alkyl and R⁶ is an amino acid residue. In some embodiments, the amino acid is selected from the group consisting of glycine, alanine, glutamic acid and proline. In some embodiments, the linker moiety is —R³C(O)NHR⁴—, wherein R³ and R⁴ are independently absent or unsubstituted or substituted alkyl. In some embodiments, R³ is methylene and R⁴ is —(CH₂)₄—. In some embodiments, R³ is methylene and R⁴ is absent. In certain embodiments of Formula (B) or (B1), the linking moiety is selected from the group consisting of:

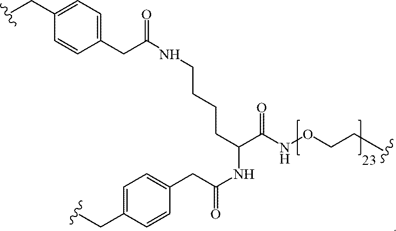

In some embodiments, m is about 3-10, about 10-15, about 15-20, about 20-25, about 25-30, about 5-16, about 15-30, about 15-25 or about 20-30. In some embodiments, m is 20, 21, 22, 23, 24 or 25.

### Protein

In some embodiments, the oligonucleotide is conjugated to a biomolecule (such as a polypeptide, protein, antibody, or antigen-binding fragment thereof) via one or more glutamine residues. In certain embodiments, the oligonucleotide is conjugated to a protein via one or more glutamine residues. In some embodiments, each glutamine residue independently is part of the sequence of the protein or is part of a Q-tag peptide sequence attached to the protein. In some embodiments, the Q-tag is comprises a glutamine residue which is linked to the rest of the conjugate. In some embodiments, the Q-tag is LLQGG or GGGLLQGG. In some embodiments, the protein is a protein fragment, a peptide or a Fc-fusion protein. In some embodiments, the protein is an antibody. In some embodiments, the protein is an antibody, e.g., a monoclonal antibody. In some embodiments, the antibody is an antibody fragment selected from the group consisting of Fab, F(ab′)2, Fab′-SH, Fv, scFv, single domain, single heavy chain, and single light chain antibody fragments. In some embodiments, the antibody is a human, humanized, or chimeric antibody. In some embodiments, the antibody comprises an Fc region. In some embodiments, the antibody comprises a human Fc region, e.g., a human IgG1, human IgG2, human IgG3, or human IgG4 antibody. In some embodiments, the antibody binds to human CD22. In some embodiments, the antibody is an anti-CD22 antibody. In some embodiments, the antibody is a human or humanized anti-CD22 antibody. In some embodiments, the antibody is an anti-CD22 antibody (e.g., RFB4, EPRA, 10F4, m971). In some embodiments, the antibody comprises a light chain variable domain (VL) and a heavy chain variable domain (VH). In some embodiments, the glutamine residue is within the C-terminus of the heavy chain of the antibody or the Q-tag peptide sequence comprising the glutamine residue is attached at the C-terminus of the heavy chain of the antibody. In some embodiments, the glutamine residue or the Q-tag peptide sequence comprising the glutamine residue is within the Fc domain of the antibody. In some embodiments of Formula (B) or (B1), the glutamine residues Q and Q′ are on the antibody at different points or the Q-tag peptide sequences comprising the glutamine residues Q and Q′ are attached to the antibody at different points. In some embodiments of Formula (B) or (B1), the glutamine residues Q and Q′ are on the C terminal of the heavy chain of the antibody or within the Fc domain of the antibody. In other embodiments, the Q-tag peptide sequences comprising Q and Q′ are independently attached to the C terminal of the heavy chain of the antibody or within the Fc domain of the antibody. In some embodiments, the glutamine residue Q or Q′ is within the light chain of the antibody. In some embodiments, the Q-tag peptide sequence comprising Q or Q′ is attached to the light chain of the antibody. In some embodiments, the glutamine residue Q or Q′ is naturally occurring. For example, mutation of N297 to N297A exposes Q295 of the antibody, where the conjugation could occur. In some embodiments of Formula (B) or (B1), the Q-tag peptide sequences comprising Q and Q′ are the same.

In some embodiments, the Q-tag comprises one or more sequences shown in Table 3.

TABLE 3

Q-tag Peptide Sequences

| SEQ ID NO. | Peptide Sequences |
|---|---|
| 6 | LLQGG |
| 7 | LSLSPGLLQGG-OH |
| 8 | WPAQGPT |
| 9 | WPQGPT |
| 10 | WAPQGPT |
| 11 | WAQGPT |
| 12 | TPGQAPW |
| 13 | PNPQLPF |
| 14 | LSQSKVLG |
| 15 | WGGQLL |
| 16 | WALQRPHYSYPD |
| 17 | WALQRPYTLTES |
| 18 | WALQGPYTLTES |

In some embodiments, the conjugate provided herein is to a target specific cell and tissue in a body for targeted delivery of a conjugated payload polynucleotide. In certain embodiments, the cell targeted by the conjugate provided herein is a natural killer cell. In certain embodiments, the cell targeted by the conjugate provided herein is myeloid cell. In certain embodiments, the cell targeted by the conjugate provided herein is B cell or T cell. In certain embodiments, the cell targeted by the conjugate provided herein is a neutrophil. In certain embodiments, the cell targeted by the conjugate provided herein is a monocyte. In certain embodiments, the cell targeted by the conjugate provided herein is a macrophage. In certain embodiments, the cell targeted by the conjugate provided herein is a dendritic cell (DC). In certain embodiments, the cell targeted by the conjugate provided herein is a mast cell. In certain embodiments, the cell targeted by the conjugate provided herein is a tumor-associated macrophage (TAM). In certain embodiments, the cell targeted by the conjugate provided herein is a myeloid-derived suppressor cell (MDSC).

In some embodiments, an antibody or conjugate of the present disclosure can delivered as a naked protein-drug conjugate, or as a protein-drug conjugate formulated with a carrier and delivered, e.g., as encapsulated or as part of a nanocarrier, nanoparticle, liposome, polymer vesicle, or viral envelope. In some embodiments, an antibody or conjugate of the present disclosure can delivered intracellularly, e.g., by conjugation to a protein-transduction domain or mimic. In some embodiments, an antibody or conjugate of the present disclosure can delivered by electroporation or microinjection.

In some embodiments, a conjugate of the present disclosure targets more than one population or type of cell, e.g., from those described supra. In some embodiments, a conjugate of the present disclosure targets both B-cells and monocytes. In some embodiments, a conjugate of the present disclosure targets both B-cells, monocytes and/or DCs. In some embodiments, a conjugate of the present disclosure targets both NKs and DCs.

In certain embodiments, the antigen-binding moiety in the conjugate provided herein is an antibody or an antigen-binding fragment thereof (e.g., F(ab)₂ or Fab) or an engineered derivative thereof (e.g., Fcab or a fusion protein (e.g., scFv)). In certain embodiments, the antigen-binding moiety in the conjugate provided herein is a human or chimeric (e.g., humanized) antibody.

In some embodiments, the antibodies or conjugates target one or more type(s) of normal cell selected from T cells, B cells, natural killer cells, neutrophils, mast cells, macrophages, antigen-presenting cells (APC), basophils, and eosinophils. In some embodiments, the antibodies or conjugates target a normal APC. In some embodiments, the antibodies or conjugates target one or more type(s) of normal APC selected from B cells, monocytes, dendritic cells, Langerhans cells, keratinocytes, endothelial cells, astrocytes, fibroblasts, and oligodendrocytes. In some embodiments, the antibodies or conjugates target a normal B cell. In some embodiments, the antibodies or conjugates target a normal dendritic cell. In some embodiments, the antibodies or conjugates target a normal macrophage. In some embodiments the antibodies or conjugates targeting one or more type(s) of normal cells do not target an abnormal cell, such as a cancer cell.

In some embodiments, an antibody or conjugate of the present disclosure can comprise a multispecific (e.g., bispecific) antibody. For example, in some embodiments, an antibody of the present disclosure is a bispecific antibody comprising 2 antigen-binding domains that bind different targets expressed on B cells. In some embodiments, an antibody of the present disclosure is a bispecific antibody comprising an antigen-binding site that binds a target expressed on a B cell and an antigen-binding site that binds a target expressed on another cell (e.g., a monocyte).

In certain embodiments, the antibody binds to an antigen expressed by a B cell. Exemplary antigens expressed by B cells that can be targeted by the conjugates provided herein include, but are not limited to, B220/CD45R, B7-1/CD80, B7-2/CD86, BCMA/TNFRSF17, BLIMP1/PRDM1, C1q R1/CD93, CD117/c-kit, CD11b/Integrin alpha M, CD19, CD1c/BDCA-1, CD1d, CD20, CD21, CD23/Fc epsilon RII, CD24, CD25/IL-2 R alpha, CD27/TNFRSF7, CD34, CD37, CD38, CD40/TNFRSF5, CD43, CD5, CD69, CD72, CD83, CXCR4, CXCR5, DEP-1/CD148, EMMPRIN/CD147, FCRL3/FcRH3, Flt-3/Flk-2, HLA-DR, IgM, IL-10, IL-12 R beta 2, IL-12/IL-35 p35, IL-21, IL-21 R, IL-27 R alpha/WSX-1/TCCR, IL-27/IL-35 EBI3 Subunit, IL-3 R alpha/CD123, IL-4 R alpha, IL-7 R alpha/CD127, IRF4, MHC class II (I-A/I-E), Neprilysin/CD10, Pax5/BSAP, Sca-1/Ly6, Siglec-2/CD22, STAT1, STAT3, Syndecan-1/CD138, TACI/TNFRSF13B, TGF-beta, TIM-1/KIM-1/HAVCR, TLR4. In particular embodiments, B cell specific antigens are selected from CD1, CD2, CD5, CD9, CD11, CD17, CD18, CD19, CD20, CD21/CD35, CD22, CD23, CD24, CD25, CD27, CD30, CD38, CD40, CD45R/B220, CD69, CD70, CD78, CD79a (Igα), CD79b (Igβ), CD80, CD86, CD93 (C1Rqp), CD137/4-1BB, CD138, CD252/OX40L, CD267, CD268/BAFF-R, CD279/PD1, CD319, PDL-2, Pax-5, IgD, IgM, Notch 2, and TLR4.

In certain embodiments, the antibody binds to an antigen expressed by a dendritic cell. Exemplary antigens expressed by a dendritic cell that can be targeted by the conjugates provided herein include, but are not limited to, B220/CD45R, BATF3, BST-2/Tetherin, CD11b/Integrin alpha M, CD11c, CD14, CD163, CD19, CD1c/BDCA-1, CD1d1, CD20, CD3, CD4, CD8, CLEC9a, CX3CR1, DC-SIGN/CD209, DEC-205/CD205, DLEC/CLEC4C/BDCA-2, E-Cadherin, EpCAM/TROP1, F4/80, Fc epsilon RI alpha, Fc gamma RI/CD64, Fc gamma RIA/CD64, Fc gamma RIB/CD64, Fc gamma RIII (CD16), Fc gamma RIIIA/CD16a, Fc gamma RIIIB/CD16b, FLT3, GFI-1, HLA-DR, IFN-alpha, IFN-beta, IFN-gamma, IGSF4A/SynCAM1, Ikaros, IL-1 beta/IL-1F2, IL-10, IL-12, IL-2, IL-23, IL-3 R alpha/CD123, IL-6, iNOS, Integrin alpha E/CD103, IRF4, IRF8, Langerin/CD207, Ly-6G (Gr-1), Ly-6G/Ly-6C (Gr-1), MHC class II (I-A/I-E), MMR/CD206, NCAM-1/CD56, Neuropilin-1, NFIL3/E4BP4, Nitric Oxide, PU.1/Spi-1, SIRP alpha/CD172a, Spi-B, Thrombomodulin/BDCA-3, TLR7, TLR9, TNF-alpha, TREM2, and XCR1. In particular embodiments, dendritic cell specific antigens are selected from CD1a, CD1b/c, CD4, CD8, CD11b, CD11c, CD40, CD45R/B220, CD49d, CD80, CD83, CD85a, CD85f, CD85g/ILT7, CD85i, CD85j, CD86, CD123, CD197/CCR7, CD205, CD206, CD207, CD208, CD209, CD273/B7-DC/PD-L2, CD303/BDCA-2, CD304/neuropilin-1, DC marker/33D1, F4/80, MHC class I, fascin, HLA-DR and Siglec H. In particular embodiments, dendritic cell specific antigens are plasmacytoid dendritic cell antigens selected from CD1a, CD1b, CD1c, CD4, CD8, CD11b, CD11c, CD40, CD45R/B220, CD49d, CD80, CD83, CD85g/ILT7, CD86, CD123, CD197 (CCR7), CD273 (B7-DC, PD-L2), CD303 (BDCA-2), CD304 (Neuropilin-1), DC Marker (33D1), F4/80, HLA-DR, MHC Class II, and Siglec H.

In certain embodiments, the antibody binds to an antigen expressed by a macrophage. Exemplary antigens expressed by a macrophage that can be targeted by the conjugates provided herein include, but are not limited to, Activin A, AIF-1/Iba1, Arginase 1/ARG1, B7-1/CD80, B7-2/CD86, Calcitonin R, CCL1/I-309/TCA-3, CCL11/Eotaxin, CCL14/HCC-1/HCC-3, CCL15/MIP-1 delta, CCL16/HCC-4, CCL17/TARC, CCL18/PARC, CCL19/MIP-3 beta, CCL2/JE/MCP-1, CCL20/MIP-3 alpha, CCL22/MDC, CCL23/Ck beta 8-1, CCL23/MPIF-1, CCL24/Eotaxin-2/MPIF-2, CCL26/Eotaxin-3, CCL3/CCL4, CCL3/MIP-1 alpha, CCL4/MIP-1 beta, CCL5/RANTES, CCL8/MCP-2, CCR2, CCR5, CD11b/Integrin alpha M, CD11c, CD15/Lewis X, CD163, CD200 R1, CD200R1L, CD36/SR-B3, CD43, CD45, CD68/SR-D1, CLEC10A/CD301, COX-2, CX3CL1/Fractalkine, CX3CR1, CXCL1/GRO alpha/KC/CINC-1, CXCL10/IP-10/CRG-2, CXCL11/I-TAC, CXCL13/BLC/BCA-1, CXCL16, CXCL2/GRO beta/MIP-2/CINC-3, CXCL3/GRO gamma/CINC-2/DCIP-1, CXCL5/ENA-70, CXCL5/ENA-74, CXCL5/ENA-78, CXCL9/MIG, CXCR1/IL-8 RA, CXCR2/IL-8 RB, DC-SIGN/CD209, DEC-205/CD205, Dectin-1/CLEC7A, Dectin-2/CLEC6A, EMR1, F4/80, Fc epsilon RI alpha, Fc gamma RI/CD64, Fc gamma RIA/CD64, Fc gamma RIB/CD64, Fc gamma RII/CD32, Fc gamma RIII (CD16), FIZZ1/RELM alpha, Galectin-3, GATA-6, G-CSF, GITR Ligand/TNFSF18, GM-CSF, HLA-DR, ID2, IFN-gamma, IFN-gamma R1/CD119, IL-1 beta/IL-1F2, IL-1 RII, IL-10, IL-15, IL-17/IL-17A, IL-18/IL-1F4, IL-1ra/IL-1F3, IL-23, IL-4 R alpha, IL-6, IL-8/CXCL8, iNOS, Integrin alpha L/CD1 1a, IRF4, IRF5, LAMP-2/CD107b, Langerin/CD207, LILRB4/CD85k/ILT3, L-Selectin/CD62L, LXR alpha/NR1H3, Ly-6G (Gr-1), Ly-6G/Ly-6C (Gr-1), MARCO, M-CSF R/CD115, Mer, MERTK, MFG-E8, MHC class II (I-A/I-E), MMR/CD206, NFATC1, NGFI-B alpha/Nur77/NR4A1, PPAR delta/NR1C2, PPAR gamma/NR1C3, RANK/TNFRSF11A, RUNX3/CBFA3, Siglec-1/CD169, Siglec-3/CD33, Siglec-F, SIGNR1/CD209b, SIRP alpha/CD172a, SLAM/CD150, SOCS-3, Sphingosine Kinase 1/SPHK1, Sphingosine Kinase 2/SPHK2, SR-AI/MSR, SR-BI, STAT1, STAT6, TGF-beta, TIM-4, TLR1, TLR2, TLR4, TLR8, TNF-alpha, TRACP/PAP/ACP5, TREM1, VCAM-1/CD106, VEGF, and YM1/Chitinase 3-like 3. In particular embodiments, macrophage specific antigens are selected from CD11a, CD11b, CD11c, CD14, CD15 (SSEA-1), CD16/32, CD33, CD64, CD68, CD80, CD85k (ILT3), CD86, CD105 (Endoglin), CD107b, CD115, CD163, CD195 (CCR5), CD282 (TLR2), CD284 (TLR4), F4/80, GITRL, HLA-DR, Mac-2 (Galectin-3), MHC Class II.

In certain embodiments, the antibody binds to an antigen expressed by an NK cell. Exemplary antigens expressed by a NK cell and can be targeted by the conjugated provided herein include, but are not limited to, CD11b, CD11c, CD16/32, CD49b, CD56 (NCAM), CD57, CD69, CD94, CD122, CD158 (Kir), CD161 (NK-1.1), CD180, CD244 (2B4), CD314 (NKG2D), CD319 (CRACC), CD328 (Siglec-7), CD335 (NKp46), Ly49, Ly108, Vα24-Jα18 TCR (iNKT), granulysin, granzyme, perforin, SIRP-α, LAIR1, SIGLEC-3 (CD33), SIGLEC-7, SIGLEC-9, LIR1 (ILT2, LILRB1), NKR-P1A (KLRB1), CD94-NKG2A, KLRG1, KIR2DL5A, KIR2DL5B, KIR2DL1, KIR2DL2, KIR2DL3, KIR2DS2, KIR2DS3, KIR2DS4, KIR2DS5, KIR3DS1, KIR2DS1, CD94-NKG2C/E, NKG2D, CD160 (BY55), CD16 (FcγRIIIA), NKp46 (NCR1), NKp30 (NCR3), NKp44 (NCR2), DNAM1(CD226), CRTAM, CD2, CD7, CD11a, CD18, CD25, CD27, CD28, NTB-A (SLAMF6), PSGL1, CD96 (Tactile), CD100 (SEMA4D), NKp80 (KLRF1, CLEC5C), SLAMF7 (CRACC, CS1, CD319), and CD244 (2B4, SLAMF4).

In certain embodiments, the antibody binds to an antigen expressed by a myeloid cell. Exemplary antigens expressed by a myeloid cell and can be targeted by the conjugated provided herein include, but are not limited to, siglec-3, siglec 7, siglec 9, siglec 10, siglec 15, CD200, CD200R, LILRB1, LILRB2, LILRB3, LILRB4, LILRB5, M-CSF, CSF-1R, GM-CSF R, IL4 R, arginase, IDO, TDO, MPO, EP2, COX-2, CCR2, CCR-7, CXCR1, CX3CR1, CXCR2, CXCR3, CXCR4, CXCR7, c-Kit, CD244, L-selectin/CD62L, CD11b, CD11c, CD68, CD163, CD204, DEC205, IL-1R, CD31, SIRPα, SIRPβ, PD-L1, CEACAM-8/CD66b, CD103, BDCA-1, BDCA2. BDCA-4, CD123, and ILT-7.

In certain embodiments, the antibody binds to an antigen expressed by an MDSC. Exemplary antigens expressed by an MDSC and can be targeted by the conjugated provided herein include, but are not limited to, siglec-3, Siglec 7, siglec 9, siglec 10, siglec 15, CD200, CD200R, LILRB1, LILRB2, LILRB3, LILRB4, LILRB5, M-CSF, CSF-1R, GM-CSF R, IL4 R, arginase, IDO, TDO, MPO, EP2, COX-2, CCR2, CCR-7, CXCR1, CX3CR1, CXCR2, CXCR3, CXCR4, CXCR7, c-Kit, CD244, L-selectin/CD62L, CD11b, CD11c, CD68, CD163, CD204, DEC205, IL-1R, CD31, SIRPα, SIRPβ, PD-L1, CEACAM-8/CD66b, CD103, BDCA-1, BDCA2. BDCA-4, CD123, and ILT-7.

In certain embodiments, the antibody binds to an antigen expressed by a TAM. Exemplary antigens expressed by a TAM and can be targeted by the conjugated provided herein include, but are not limited to, siglec-3, Siglec 7, siglec 9, siglec 10, siglec 15, CD200, CD200R, LILRB1, LILRB2, LILRB3, LILRB4, LILRB5, M-CSF, CSF-1R, GM-CSF R, IL4 R, arginase, IDO, TDO, MPO, EP2, COX-2, CCR2, CCR-7, CXCR1, CX3CR1, CXCR2, CXCR3, CXCR4, CXCR7, c-Kit, CD244, L-selectin/CD62L, CD11b, CD11c, CD68, CD163, CD204, DEC205, IL-1R, CD31, SIRPα, SIRPβ, PD-L1, CEACAM-8/CD66b, CD103, BDCA-1, BDCA2. BDCA-4, CD123, and ILT-7.

In certain embodiments, the antibody binds to an antigen specific to a NK cell. In certain embodiments, an NK cell is targeted by an anti-CD56 antibody. In certain embodiments, the antibody is an anti-CD56 antibody. In certain embodiments, the antibody is a monoclonal anti-CD56 antibody. In certain embodiments, the antibody is a murine anti-CD56 antibody. In certain embodiments, the murine anti-CD56 antibody is clone 5.1H11 (BioLegend, Cat No: 362502). In certain embodiments, the murine anti-CD56 antibody is clone MEM-188 (BioLegend, 304601). In certain embodiments, the murine anti-CD56 antibody is clone QA17A16 (BioLegend, Cat No: 392402). In certain embodiments, the antibody is a humanized anti-CD56 antibody. In certain embodiments, the antibody is a human anti-CD56 antibody. In certain embodiments, the antibody is a humanized anti-CD56 antibody

B cells can be targeted by anti-CD38, anti-CD79b, anti-CD30, anti-CD22, or anti-CD20, anti-CD19 antibodies or antigen-binding fragments thereof or engineered derivatives thereof. Plasmacytoid dendritic cells (pDCs) can be targeted by anti-DEC205, anti-CD304 (BDCA4), anti-CD303 (BDCA2), anti-CD40, anti-CD74, or anti-CD123 antibodies or antigen-binding fragments thereof or engineered derivatives thereof. Macrophages can be targeted by anti-CD163, anti-CD40, anti-CD74, anti-CD206, or anti-CD123 antibodies or antigen-binding fragments thereof or engineered derivatives thereof. In some embodiments, a conjugate of the present disclosure comprises an immunomodulating oligonucleotide as described herein conjugated to a polypeptide, carbohydrate, or other compound that associates with or binds a target antigen described herein (e.g., CD22, a B cell antigen, a macrophage antigen, and so forth).

Non-limiting examples of anti-CD38 antibodies are daratumumab, SAR650984, MOR202, or any one of antibodies Ab79, Ab19, Ab43, Ab72, and Ab110 disclosed in WO 2012/092616, the disclosure of these antibodies is incorporated herein by reference. A non-limiting example of an anti-CD79b antibody is huMA79b v28 disclosed in WO 2014/011521. A non-limiting example of an anti-CD22 antibody is 10F4 disclosed in US 2014/0127197. A non-limiting example of an anti-CD20 antibody is rituximab. A non-limiting example of an anti-DEC205 antibody is provided in US 2010/0098704, the antibodies of which are incorporated herein by reference. Non-limiting examples of anti-CD40 antibodies are lucatumumab and dacetuzumab. A non-limiting example of an anti-CD304 antibody is vesencumab.

In some embodiments, the antibody is selected from the group consisting of an anti-CD20 antibody, anti-CD22 antibody, anti-CD30 antibody, anti CD37 antibody, anti-CD38 antibody, anti-CD40 antibody, anti-CD74 antibody, anti-CD79b antibody, anti-CD205 antibody, anti-CD274 antibody, anti-CD303 antibody, anti-CD304 antibody, anti-CD19 antibody, anti-CD1 antibody, anti-CD2 antibody, anti-CD3 antibody, anti-CD5 antibody, anti-CD6 antibody, anti-CD9 antibody, anti-CD11 antibody, anti-CD18 antibody, anti-CD21 antibody, anti-CD23 antibody, anti-CD24 antibody, anti-CD25 antibody, anti-CD26 antibody, anti-CD44 antibody, anti-CD45R antibody, anti-CD49 antibody, anti-CD66 (Carcinoembrionic antigen, CEA) antibody, anti-CD93 antibody, anti-CD52 antibody, anti-CD56 antibody, anti-CD123 antibody, anti-CD138 antibody, anti-CD163 antibody, anti-SLAMF7 antibody, anti-CD180 antibody, anti-DEC205 antibody, and anti-CD206 antibody. In some embodiments, the antibody is an anti-CD20 antibody. In some embodiments, the antibody is an anti-CD22 antibody.

In some embodiments, the CpG-Ab immunoconjugate specifically binds to a tumor-associated antigen of the cancer being treated by the present method. Examples of tumor-associated antigens (TAAs) that can be targeted by the CpG-Ab immunoconjugate of the present disclosure include, but are not limited to, sequences comprising all or part of the sequences of AMHR2, NTSE, FLT1, nectin 4, 5T4 MT-1/MMP-14, DKK1, myostatin, sema4D, Trop-2, HER3, HER2, HER2/neu, HER1, VWF, IGF-1, GRP78, CXCR4, cMET, vitmentin, VEGFR2, VEGFR1, VEGF, VEGF-A, TYRP1 (glycoprotein 75), TWEAK receptor, tumor antigen CTAA16.88, TRAIL-R2, TRAIL-R1, TNF-alpha, TGF-beta, TGF beta 2, TGF beta 1, TFPI, tenascin C, TEM1, TAG-72, STEAP1, sphingosine-1-phosphate, SOST, SLAMF7, BCL-2, selectin P, SDC1, sclerostin, RTN4, RON, Rhesus factor, RHD, respiratory syncytial virus, RANKL, rabies virus glycoprotein, PDGF-R beta, phosphatidylserine, phosphate-sodium co-transporter, PDGF-R alpha, PDCD1, PD-1, PD-L1, PCSK9, oxLDL, OX-40, NRP1, Notch receptor 4, Notch receptor 3, Notch receptor 2, Notch receptor 1, NOGO-A, NGF, neural apoptosis-regulated proteinase 1, NCA-90 (granulocyte antigen), NARP-1, N-glycolylneuraminic acid, myostatin, myelin-associated glycoprotein, mucin CanAg, MSLN, MS4A1, MIF, MCP-1, LTA, LOXL2, lipoteichoic acid, LINGO-1, LFA-1 (CD11a), Lewis-Y antigen, L-selectin (CD62L), KIR, KIR ligand, ITGB2 (CD18), ITGA2, interferon receptor, interferon gamma-induced protein, integrin αvβ3, integrin αIIβ3, integrin α7β7, integrin α5β1, integrin α4β7, integrin α4, insulin-like growth factor I receptor, Influenza A hemagglutinin, ILGF2, IL9, IL6, IL4, IL3 IRA, IL23, ILI 7A, IL-6 receptor, IL-6, IL-S, IL-4, IL-23, IL-22, IL- I, IL- I 7A, IL-I 7, IL-13, IL- I2, IL- I, IL 20, IGHE, IGF-I, IGF- I receptor, IgE Fc region, IFN-gamma, IFN-alpha, ICAM-1 (CD54), human TNF, human scatter factor receptor kinase, Hsp90, HNGF, HLA-DR, HIV-1, histone complex, HHGFR, HGF, hepatitis B surface antigen, GUCY2C, GPNMB, GMCSF receptor alpha-chain, glypican 3, GD3 ganglioside, GD2, ganglioside GD2, Frizzled receptor, folate receptor 1, folate hydrolase, fibronectin extra domain-B, fibrin II, beta chain, FAP, F protein of respiratory syncytial virus, ERBB3, episialin, EpCAM, endotoxin, EGFR, EGFL7, E. coli shiga toxin type-2, E. coli shiga toxin type- I, DRS, DPP4, DLL4, dabigatran, cytomegalovirus glycoprotein B, CTLA-4, CSF2, CSF1R, clumping factor A, CLDN6, CLDN18.1, CLDN18.2, ch4DS, CFD, CEA-related antigen, CEA, CD80, CD79B, CD74, CD73, CD70, CD6, CD56, CD52, CD51, CD5, CD44 v6, CD41, CD40 ligand, CD40, CD47, CD4, CD39, CD38, CD37, CD33, CD30 (TNFRSF8), CD123, CD138, CD3 epsilon, CD3, CD28, CD274, CD27, CD2S (a chain of IL-2 receptor), CD23 (IgE receptor), CD221, CD22, CD200, CD20, CD2, CD19, CD137, CD142, CD154, CD152, CD15, CD147 (basigin), CD140a, CD125, CD11, CD-18, CCR5, CCR4, CCL11 (eotaxin-I), cardiac myosin, carbonic anhydrase 9 (CA-IX), Canis lupus familiaris IL31, CA-125, C5, C242 antigen, C-X-C chemokine receptor type 4, beta-amyloid, BAFF, B7-H3, B-lymphoma cell, AOC3 (VAP-I ), anthrax toxin, protective antigen, angiopoietin 3, angiopoietin 2, alpha-fetoprotein, AGS-22M6, adenocarcinoma antigen, ACVR2B, activin receptor-like kinase I, 5T4, 5AC, 4- IBB, 1-40-beta-amyloid, EGFR, EGFRvIII, gp100 or Pmel17, CEA, MART-1/Melan-A, MAGE-A1, MAGE-A2, MAGE-A3, MAGE-A4, MUC-1, GPNMB, HMW-MAA, TIM1, ROR1, CD19, gp100, Dipeptidyl peptidase IV (DPPIV), adenosine deaminase-binding protein (ADAbp), cyclophilin b, Colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family (e.g. MUC1, MUC16, etc; see e.g. U.S. Pat. No. 6,054,438; WO98/04727; or WO98/37095), p21ras, RCAS1, alpha-fetoprotein, E-cadherin, alpha-catenin, beta-catenin and gamma-catenin, p120ctn, PRAME, NY-ESO-1, cdc27, adenomatous polyposis coli protein (APC), fodrin, Connexin 37, Ig-idiotype, p15, gp75, GM2 and GD2 gangliosides, Smad family of tumor antigens brain glycogen phosphorylase, SSX-1, SSX-2 (HOM-MEL-40), SSX-1, SSX-4, SSX-5, SCP-1 and CT-7, and c-erbB-2 and viral antigens such as the HPV-16 and HPV-18 E6 and E7 antigens and the EBV-encoded nuclear antigen (EBNA)-1, βhCG, WT1, TRP-2, NY-BR-1, NY-CO-58, MN (gp250), Telomerase, and germ cell derived tumor antigens. Tumor-associated antigens also include the blood group antigens, for example, Lea, Leb, LeX, LeY, H-2, B-1, B-2 antigens. Tumor-associated antigen can be identified using methods known in the art, such as disclosed in Zhang et al. Supra.

Particularly, in some embodiments, the CpG-Ab immunoconjugate specifically binds to a tumor-associated antigen selected from CD19, CD20, CD22, CD25, CD30, CD33, CD38, CD40, CD44, CD45R (B220), CD49, CD52, CD56, CD70, CD74, CD79a, CD79b, CD93, CD123, CD138, CD163, CD205, CD206, CD274, CD303, and CD304, folate receptor alpha, folate receptor beta, mesothelin, PSMA, Her-2, EGFR, CLDN18.2, 5T4, CD47, nectin 4, transferrin receptor, integrin, cripto, EphA2, AGS-5, AGS-16, CanAg, EpCAM, IL4 receptor, IL2 receptor, Lewis Y, GPNMB and Trop2.

In certain embodiments, the anti-CD22 antibody is an antibody comprising a VH and VL as shown below in Table 4.

TABLE 4

| Name | Domain | SEQ ID NO. | Sequence |
|---|---|---|---|
| mCD22Q | VH | 19 | QVQLQQPGAEIVRPGTSVKLSCKASGYTFTDY WMNWVKQRPGQGLEWFGAIDPSDSYTRYNQE FKGKATLTVDTSSTTAYMQLSSLTSEDSAVYFC ARSDYTYSFYFDYWGLGTTLTVSS |
| mCD22Q | VL | 20 | DIVMTQAAFSNPVTLGTSASISCRSSKSLLHSNG ITYLYWYLQKPGQSPQLLIYQMSNLASGVPDRF SSSGSGTDFTLRISRVEAEDVGVYYCAQNLELP WTFGGGTKLEIK |
| 10F4 | VH | 21 | EVQLVESGGGLVQPGGSLRLSCAASGYEFSRS WMNWVRQAPGKGLEWVGRIYPGDGDTNYSG KFKGRFTISADTSKNTAYLQMNSLRAEDTAVY YCARDGSSWDWYFDVWGQGTLVTVSS |
| 10F4 | VL | 22 | DIQMTQSPSSLSASVGDRVTITCRSSQSIVHSVG NTFLEWYQQKPGKAPKLLIYKVSNRFSGVPSRF SGSGSGTDFTLTISSLQPEDFATYYCFQGSQFPY TFGQGTKVEIK |

### III. Methods of Conjugation

Provided herein are methods for preparing a conjugate comprising a protein (e.g.,an antibody or antigen-binding fragment thereof) and one or more immunomodulating oligonucleotides linked via one or more glutamine residues as shown in the structure of Formula (A), (A1), (A2), (B), or (B1). In some embodiments, the methods comprise combining an antibody comprising at least one exposed glutamine residue (wherein the glutamine residue may be part of the sequence of the antibody or part of a Q-tag peptide sequence attached to the antibody) and an oligonucleotide under conditions sufficient to induce conjugation, i.e., amidation reaction between the CpG and glutamine residue. In other embodiments, the methods comprise reacting an antibody comprising at least one exposed glutamine residue (wherein the glutamine residue may be part of the sequence of the antibody or part of a Q-tag peptide sequence attached to the antibody) and an oligonucleotide under chemical conditions sufficient to induce conjugation. In still other embodiments, the methods comprise reacting an antibody comprising at least one exposed glutamine residue (wherein the glutamine residue may be part of the sequence of the antibody or part of a Q-tag peptide sequence attached to the antibody) and an oligonucleotide under enzymatic conditions, e.g., with transglutaminase, sufficient to induce conjugation.

### Transglutaminase-Mediated Conjugation Reaction Conditions

Provided herein are methods for preparing a conjugate of Formula (A1):

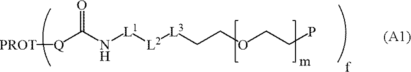

or a stereoisomer, a mixture of two or more diastereomers, a tautomer, or a mixture of two or more tautomers thereof; or a pharmaceutically acceptable salt, solvate, or hydrate thereof; comprising reacting (1) a compound of Formula (I):

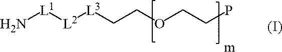

or a stereoisomer, a mixture of two or more diastereomers, a tautomer, or a mixture of two or more tautomers thereof; or a pharmaceutically acceptable salt, solvate, or hydrate thereof, and (2) a protein comprising one or more glutamine residues in the presence of a transglutaminase, wherein:
Q is a glutamine residue, wherein each glutamine residue independently is part of the sequence of PROT or is part of a Q-tag peptide sequence attached to PROT;
PROT is a protein connected to the rest of the conjugate via one or more glutamine residues Q;
each L¹ is independently unsubstituted or substituted alkyl,
each L² is unsubstituted or substituted aryl, or unsubstituted or substituted heteroaryl,
each L³ is independently absent or a linker moiety,
f is an integer selected from the group consisting of 1-20,
m is an integer selected from the group consisting of 0-50, and
P is an immunomodulating oligonucleotide.

Also provided herein is a method of preparing a conjugate of Formula (B1):

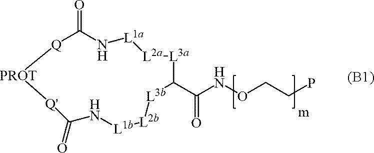

or a stereoisomer, a mixture of two or more diastereomers, a tautomer, or a mixture of two or more tautomers thereof; or a pharmaceutically acceptable salt, solvate, or hydrate thereof;comprising reacting (1) a compound of Formula (II):

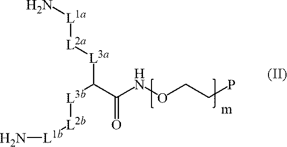

or a stereoisomer, a mixture of two or more diastereomers, a tautomer, or a mixture of two or more tautomers thereof; or a pharmaceutically acceptable salt, solvate, or hydrate thereof; and (2) a protein comprising two or more glutamine residues in the presence of a transglutaminase, wherein:
Q and Q′ are each a glutamine residue, wherein each glutamine residue independently is part of the sequence of PROT or is part of a Q-tag peptide sequence attached to PROT;
PROT is a protein connected to the rest of the conjugate via Q and Q′;
L^{1a} and L^{1b} are independently unsubstituted or substituted alkyl,
L^{2a} and L^{2b} are independently absent, unsubstituted or substituted alkyl, unsubstituted or substituted aryl, or unsubstituted or substituted heteroaryl,
L^{3a} and L^{3b} are independently absent or a linker moiety,
m is an integer selected from the group consisting of 0-50, and
P is an immunomodulating oligonucleotide.

Any conjugates of Formula (A), (A1), (A2), (B), or (B1) as described herein can be prepared using methods described herein.

In some embodiments, the conjugate comprises one or more, two or more, three or more, four or more, five or more, six or more, seven or more, eight or more, nine or more, ten or more, or twenty or more glutamine residues Q. In some embodiments, the conjugate comprises one, two, three, four, five, six, seven, eight, nine, ten, or twenty glutamine residues Q. In some embodiments, the conjugate has 2 glutamine residues Q. In some embodiments, the conjugate comprises one or more, two or more, three or more, four or more, five or more, six or more, seven or more, eight or more, nine or more, ten or more, or twenty or more Q-tag peptides. In some embodiments, the conjugate comprises one, two, three, four, five, six, seven, eight, nine, ten, or twenty Q-tag peptides. In some embodiments, the conjugate has 2 Q-tag peptides. In some embodiments, the conjugate comprises one or more, two or more, three or more, four or more, five or more, six or more, seven or more, eight or more, nine or more, ten or more, or twenty or more immunomodulating oligonucleotides. In some embodiments, the conjugate comprises one, two, three, four, five, six, seven, eight, nine, ten, or twenty immunomodulating oligonucleotides. In some embodiments, the conjugate has one immunomodulating oligonucleotide.

In another aspect, the method comprises reacting a compound of Formula (I) or Formula (II) and a protein, such as an antibody, comprising one or more glutamine residues in the presence of a transglutaminase. In some embodiments, the final concentration of the compound of Formula (I) or Formula (II) is in the range of about 1-100 µM. In some embodiments, the final concentration of the glutamine- (or Q-tag) comprising antibody is in the range of about 1-500 µM. In some embodiments, the final concentration of transglutaminase is in the range of about 1-500 µM. In some embodiments, the final concentration of transglutaminase is in the range of about 1-50 µM, about 50-100 µM, about 100-150 µM, about 150-200 µM, about 200-250 µM, about 250-300 µM, about 300-400 µM, about 400-500 µM, about 100-125 µM, about 125-150 µM, about 150-175 µM, about 175-200 µM, about 200-225 µM, about 225-250 µM, about 250-275 µM, about 275-300 µM, about 300-325 µM or about 325-350 µM.

In some embodiments, the ratio of the protein and the compound of Formula (I) or Formula (II) is in the range of about 1:1-250:1, about 1:1-10:1, about 1:1-5:1, about 5:1-10:1, about 10:1-20:1, about 20:1-30:1, about 30:1-40:1, about 40:1-50:1, about 50:1-75:1, about 75:1-100:1, about 100:1-150:1, about 150:1-200:1, about 200:1-250:1, about 1:1-25:1, about 25:1-50:1, about 50:1-75:1, about 75:1-100:1 or about 100:1-250:1 by molarity. In some embodiments, the ratio of the compound of Formula (I) and the protein is about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1 or about 20:1 by molarity. In some embodiments, the ratio of the compound of Formula (II) and the antibody is about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1 or about 20:1 by molarity.

In some embodiments, the molar ratio of the protein and transglutaminase (protein-to-transglutaminase) is in the range of about 1:1-500:1, about 1:1-5:1, about 5:1-20:1 about 5:1-10:1, about 10:1-20:1, about 10:1-18:1, about 20:1-30:1, about 30:1-40:1, about 40:1-50:1, about 50:1-75:1, about 75:1-100:1, about 100:1-150:1, about 150:1-200:1, about 200:1-250:1, about 1:1-25:1, about 25:1-50:1, about 50:1-75:1, about 75:1-100:1, about 100:1-150:1, about 150:1-200:1, about 200:1-250:1, about 250:1-300:1, about 300:1-400:1 or about 400:1-500:1. In some embodiments, the molar ratio of the peptide and transglutaminase (peptide-to-transglutaminase) is about 15:1, about 16:1, about 17:1, about 18:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1 or about 50:1.

In some embodiments, the ratio of Q-tag: CpG: transglutaminase is about 1:1.3:10. In some embodiments, the ratio of Q-tag: CpG: transglutaminase is about 1:1.5:10. In some embodiments, the ratio of Q-tag: CpG: transglutaminase is about 1:1.3:15.

In some embodiments, the reaction is incubated at greater than 15° C., greater than 20° C., greater than 25° C., greater than 30° C., greater than 35° C., greater than 40° C., greater than 45° C., or greater than 50° C. In some embodiments, the reaction is incubated at about room temperature. In some embodiments, the reaction is incubated for at least 10 minute, 20 minutes, 30 minutes, 45 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours or 60 hours.

In some embodiments, the method described herein produces the compound of Formula (A) at greater than about 5%, greater than about 10%, greater than about 15%, greater than about 20%, greater than about 25%, greater than about 30%, greater than about 35%, greater than about 40%, greater than about 45%, greater than about 50%, greater than about 60%, greater than about 70%, greater than about 80%, greater than about 90%, greater than about 95%, greater than about 97% or greater than about 99% as compared to the peptide.

In some embodiments, the pH of the reaction is in the range of about 4-10. In some embodiments, the pH of the reaction is in the range of about 4-6, about 6-8 or about 8-10. In some embodiments, the pH of the reaction is in the range of about 7-8.

In another aspect, reactions useful for attaching a linking moiety to an oligonucleotide are known in the art, including, but not limited to Hüisgen cycloaddition (metal-catalyzed or metal-free) between an azido and an alkyne-based conjugating group (e.g., optionally substituted C₆-₁₆ heterocyclylene containing an endocyclic carbon-carbon triple bond or optionally substituted C₈-₁₆ cycloalkynyl) to form a triazole moiety; the Diels-Alder reaction between a dienophile and a diene/hetero-diene; bond formation via pericyclic reactions such as the ene reaction; amide or thioamide bond formation; sulfonamide bond formation (e.g., with azido compounds); alcohol or phenol alkylation (e.g., Williamson alkylation), condensation reactions to form oxime, hydrazone, or semicarbazide group; conjugate addition reactions by nucleophiles (e.g., amines and thiols); disulfide bond formation; and nucleophilic substitution (e.g., by an amine, thiol, or hydroxyl nucleophile) at a carbonyl (e.g., at an activated carboxylic acid ester, such as pentafluorophenyl (PFP) ester or tetrafluorophenyl (TFP) ester) or at an electrophilic arene (e.g., SNAr at an oligofluorinated arene, a fluorobenzonitrile group, or fluoronitrobenzene group).

In certain embodiments, the attachment reaction is a dipolar cycloaddition, and the conjugation moiety includes azido, optionally substituted C₆-₁₆ heterocyclylene containing an endocyclic carbon-carbon triple bond, or optionally substituted C₈-₁₆ cycloalkynyl. The complementary reactive group and the conjugating group are selected for their mutual complementarity. For example, an azide is used in one of the conjugating group and the complementary reactive group, while an alkyne is used in the other of the conjugating group and the complementary reactive group.

### Attachment of Linking Moiety to the Oligonucleotide

A linking moiety can be attached to an oligonucleotide by forming a bond between a attaching group in the oligonucleotide and a complementary reactive group bonded to the linking moiety. In certain embodiments, the linking moiety, is modified to include a complementary reactive group. Methods of introducing such complementary reactive groups into a linking moiety is known in the art.

In certain embodiments, the complementary reactive group is optionally substituted C₂-₁₂ alkynyl, optionally substituted N-protected amino, azido, N-maleimido, S-protected thiol,

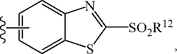

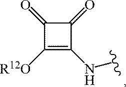

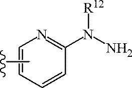

or a N-protectedmoiety thereof,

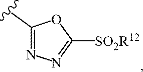

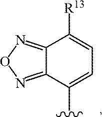

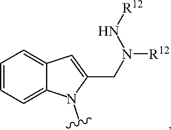

optionally substituted C₆-₁₆ heterocyclyl containing an endocyclic carbon-carbon triple bond (e.g.,

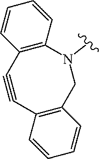

), 1,2,4,5-tetrazine group (e.g.,

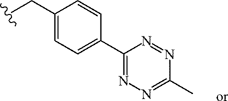

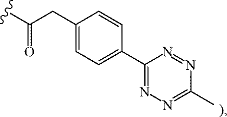

optionally substituted C₈-₁₆ cycloalkynyl (e.g.,

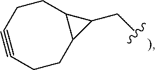

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO; wherein:
R^{N1} is H, N-protecting group, or optionally substituted C₁-₆ alkyl;
each R¹² is independently H, optionally substituted C₁-₆ alkyl, or O-protecting group (e.g., a carboxyl protecting group); and
R¹³ is halogen (e.g., F).

In certain embodiments, the complementary reactive group is protected until the conjugation reaction. For example, a complementary reactive group that is protected can include —COOR^{PGO} or —NHR^{PGN}, where R^{PGO} is an O-protecting group (e.g., a carboxyl protecting group), and R^{PGN} is an N-protecting group.

### IV. Immunomodulating Polynucleotides

Also provided herein is an immunomodulating polynucleotide comprising a nucleotide sequence selected from the group consisting of SEQ ID NOS: 1-5.

### General Polynucleotide Synthesis

### General Scheme

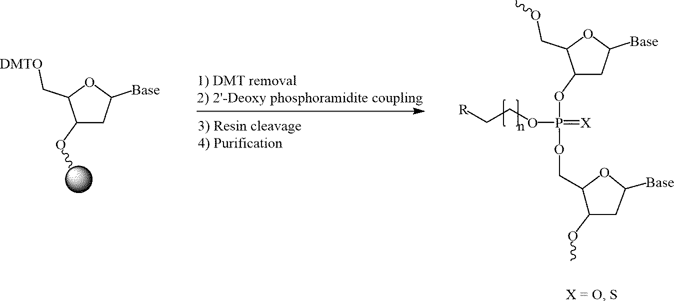

### Experimental Details:

Automated polynucleotide synthesis (1 µmol scale) was carried out on MerMade 6 or 12 with the following reagents and solvents:
Oxidizer — 0.02 M I₂ in THF/pyridine/H₂O (60 s oxidation per cycle),
Sulfurizing Reagent II — dithiazole derivative/pyridine/acetonitrile (0.05 M, in 6:4 pyridine:acetonitrile) (60 s per cycle)
Deblock — 3% trichloroacetic acid (2x 40 s deblocks per cycle),
Cap Mix A — THF/2,6-lutidine/Ac₂O (60 s capping per cycle), and
Cap Mix B — 16% methyl imidazole in THF (60 s capping per cycle)

Exceptions to standard polynucleotide synthesis conditions were as follows:
CPG supports with a non-nucleosidic linker called Uny-linker was used.
All 2′-deoxyribose-phosphoramidites were resuspended to 100 mM in 100% anhydrous acetonitrile prior to synthesis, except some of the modified 2′-deoxy-phosphoramidites were dissolved to 100 mM in THF/acetonitrile mixture (1:4) depend on the solubility of the starting material.
Phosphoramidite activation was performed with a 2.5-fold molar excess of 5-benzylthio-1H-tetrazole (BTT). Activated 2′-deoxyribose-phosphoramidites were coupled for 2× 1 minute coupling per insertion and modified phosphoramidites were coupled for 2× 3 minute coupling per insertion.
Sulfurization of the backbone was performed with 0.05 M Sulfurizing Reagent II in pyridine/acetonitrile (6:4) for 1 min.

### Polynucleotide Deprotection & Purification Protocol:

Following automated polynucleotide synthesis, solid support and base protecting groups (such as A-Bz, C-Ac, G-iBu, etc.) and methyl esters of phosphotriesters were cleaved and de-protected in 1 mL of AMA (1:1 ratio of 36% aq. ammonia and 40% methylamine in methanol) for 2 h or more at room temperature followed by centrifugal evaporation.

Crude polynucleotide pellets were resuspended in 100 µL of 50% acetonitrile, briefly heated to 65° C. and vortexed thoroughly.

For polynucleotide purification, 100 µL crude polynucleotides were injected onto RP-HPLC with the following buffers/gradient:
Buffer A = 50 mM TEAA in Water;
Buffer B = 90% Acetontrile; and
Flow Rate = 1 mL/min;
Gradient:
   0 - 2 min (100% Buffer A / 0% Buffer B),
   2 - 42 min (0% to 60% Buffer B), and
   42 - 55 min (60% to 100% Buffer B).

### DBCO Conjugation and Purification Protocol:

DBCO NHS ester was conjugated to the crude 2′-deoxy DMT-polynucleotide as described here. The crude polynucleotide pellet was suspended into 45 µL DMSO, briefly heated to 65° C. and vortexed thoroughly. 5 µL of DIPEA was added followed by DBCO-NHS ester (30 eq), which was pre-dissolved in DMSO (1 M). The reaction was allowed to stand for 10 minutes or until product formation was confirmed by MALDI. Total 80 µL of crude polynucleotide samples were injected onto RP-HPLC with the following buffers/gradient:
Buffer A = 50 mM TEAA in Water
Buffer B = 90% Acetonitrile
Flow Rate = 1 mL/min
Gradient:
   o 0 - 2 min (90% Buffer A / 10% Buffer B)
   o 2 - 42 min (0% to 60% Buffer B)
   o 42 - 55 min (60% to 100% Buffer B).

Across the dominant RP-HPLC peaks, 0.5 mL fractions were collected and analyzed by MALDI-TOF mass spectrometry to confirm presence of desired mass. Mass-selected, purified fractions were frozen and lyophilized. Once dry, fractions were resuspended, combined with corresponding fractions, frozen and lyophilized.

DMT Cleavage: lyophilized pellets were suspended in 20 µL of 50% acetonitrile and added 80 µL of acetic acid, samples were kept standing at room temperature for 1 h, frozen and lyophilized. The dried samples were re-dissolved in 20% acetonitrile and desalted through NAP 10 (Sephadex™-G25 DNA Grade) columns. Collected, pure fractions were frozen and lyophilized for final product.

### Methods for Attaching Oligonucleotides to Linking Moiety

### Cu-catalyzed Click Reaction

### Copper-THPTA Complex Preparation

A 5 mM aqueous solution of copper sulfate pentahydrate (CuSO₄-5H₂O) and a 10 mM aqueous solution of tris(3-hydroxypropyltriazolylmethyl)amine (THPTA) were mixed 1:1 (v/v) (1:2 molar ratio) and allowed to stand at room temperature for 1 hour. This complex can be used to catalyze Huisgen cycloaddition, e.g., as shown in the general conjugation schemes below.

### General Procedure (100 nM Scale):

To a solution of 710 µL of water and 100 µL tert-butanol (10% of final volume) in a 1.7 mL Eppendorf tube was added 60 µL of the copper-THPTA complex followed by 50 µL of a 2 mM solution of the oligo, 60 µL of a 20 mM aqueous sodium ascorbate solution and 20 µL of a 10 mM solution of targeting moiety-azide. After thorough mixing the solution was allowed to stand at room temperature for 1 hour. Completion of the reaction was confirmed by gel analysis. The reaction mixture is added to a screw cap vial containing 5-10 fold molar excess of SiliaMetS® TAAcONa (resin bound EDTA sodium salt). The mixture is stirred for 1 hour. This mixture is then eluted through an illustra™Nap™-10 column Sephadex™. The resulting solution is then frozen and lyophilized overnight.

### Attachment Through Amide Linkage:

Conjugation through amidation may be performed under the amidation reaction conditions known in the art. See, e.g., Aaronson et al., Bioconjugate Chem. 22:1723-1728, 2011.

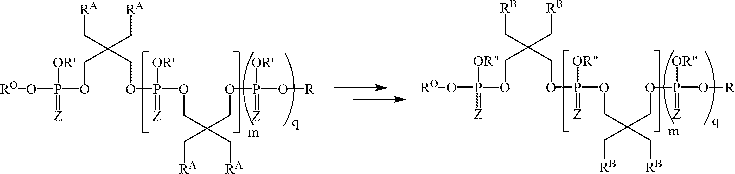

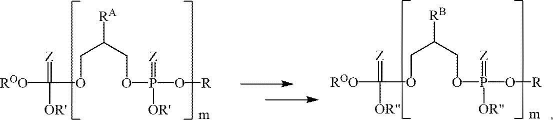

where
each q is 0 or 1;
each m is an integer from 0 to 5;
Z is O or S;
R^{O} is a bond to a nucleoside in a polynucleotide;
R is a bond to H, a nucleoside in a polynucleotide, to solid support, or to a capping group (e.g., —(CH₂)₃—OH);
each R′ is independently H, —Q¹—Q^{A1}, a bioreversible group, or a non-bioreversible group;
each R″ is independently H, —Q¹—Q^{A}—Q¹—T, a bioreversible group, or a non-bioreversible group;
each R^{A} is independently H or —OR^{C}, where R^{c} is —Q¹—Q^{A1}, a bioreversible group, or a non-bioreversible group;
each R^{B} is independently H or —OR^{D}, where R^{D} is —Q¹—Q^{A_}Q^{2_}T, a bioreversible group, or a non-bioreversible group; where
   each Q¹ is independently a divalent, trivalent, tetravalent, or pentavalent group, in which one valency is bonded to Q^{A} or Q^{A1}, the second valency is open, and each of the remaining valencies, when present, is independently bonded to an auxiliary moiety;
   each Q² is independently a divalent, trivalent, tetravalent, or pentavalent group, in which one valency is bonded to Q^{A}, the second valency is bonded to T, and each of the remaining valencies, when present, is independently bonded to an auxiliary moiety;
   Q^{A} is optionally substituted C₂-₁₂ heteroalkylene containing —C(O)—N(H)— or —N(H)—C(O)—;
   Q^{A1} is —NHR^{N1} or —COOR¹², where R^{N1} is H, N-protecting group, or optionally substituted C₁-₆ alkyl, and R¹² is H, optionally substituted C₁-₆ alkyl, or O— protecting group; and
   T is a linking moiety,
   provided that the starting materials contain at least one —Q¹—Q^{A1}, and products contain —Q¹—Q^{A}—Q²—^{T}.

Solution phase attachment:

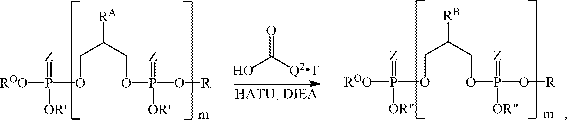

where
m is an integer from 0 to 5;
Z is O or S;
R^{O} is a bond to a nucleoside in a polynucleotide;
R is a bond to H, a nucleoside in a polynucleotide, or to a capping group;
each R′ is independently H, —Q¹—NH₂, a bioreversible group, or a non-bioreversible group;
each R″ is independently H, —Q¹—NH—CO—Q²—T, a bioreversible group, or a non-bioreversible group;
each R^{A} is independently H or —OR^{c}, where R^{c} is —Q¹—NH₂, a bioreversible group, or a non-bioreversible group;
each R^{B} is independently H or —OR^{D}, where R^{D} is —Q¹—NH—CO—Q²—T, a bioreversible group, or a non-bioreversible group; where
   each Q¹ is independently a divalent, trivalent, tetravalent, or pentavalent group, in which one valency is bonded to —NH—CO— or —NH₂, the second valency is open, and each of the remaining valencies, when present, is independently bonded to an auxiliary moiety;
   each Q² is independently a divalent, trivalent, tetravalent, or pentavalent group, in which one valency is bonded to —NH—CO—, the second valency is a bond to T, and each of the remaining valencies, when present, is independently bonded to an auxiliary moiety; and
   T is a linking moiety,
   provided that the starting material contains —Q¹—NH₂, and the product contains —Q¹—NH—CO—Q²—T.

On-support attachment:

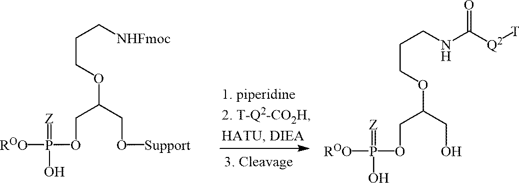

where
Z is O or S;
R^{O} is a bond to a nucleoside in a polynucleotide;
each Q² is independently a divalent, trivalent, tetravalent, or pentavalent group, in which one valency is bonded to —NH—CO—, the second valency is a bond to T, and each of the remaining valencies, when present, is independently bonded to an auxiliary moiety; and
T is a linking moiety.

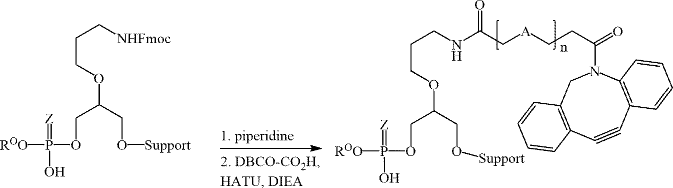

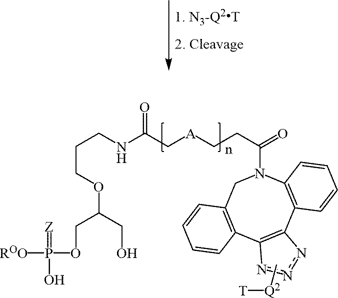

where
n is an integer from 1 to 8;
A is O or —CH₂—;
Z is O or S;
R^{o} is a bond to a nucleoside in a polynucleotide;
each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and
T is a linking moiety.

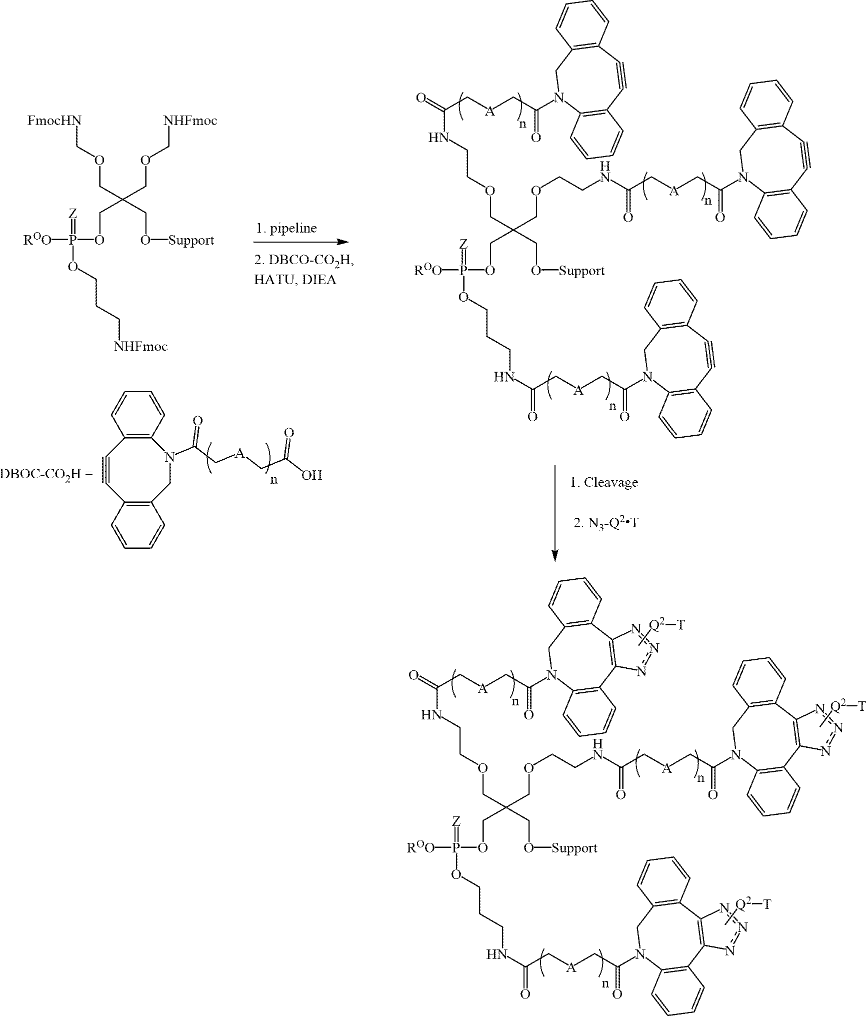

where
n is an integer from 1 to 8;
A is O or —CH₂—;
Z is O or S;
R^{O} is a bond to a nucleoside in a polynucleotide;
each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and
T is a linking moiety.

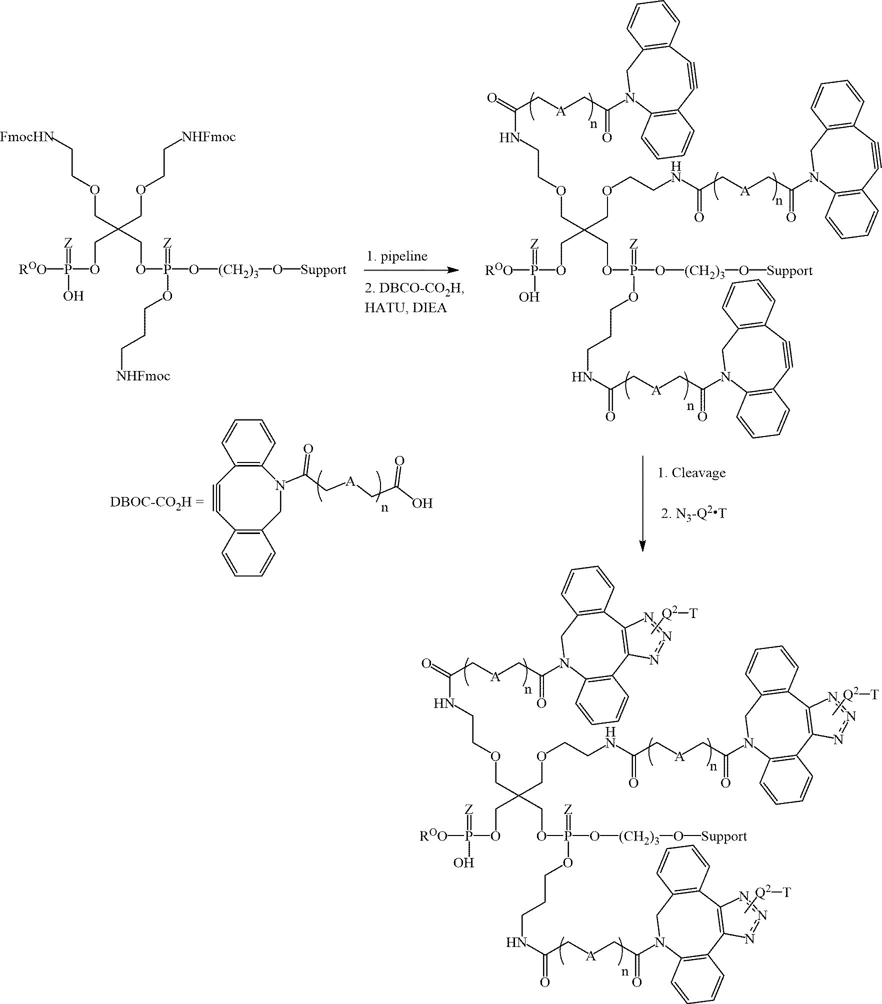

where
n is an integer from 1 to 8;
A is O or —CH₂—;
Z is O or S;
R^{O} is a bond to a nucleoside in a polynucleotide;
each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and
each T is independently a linking moiety.

### Representative Example of Fmoc Deprotection of a Phosphotriester:

A polynucleotide including a phosphotriester with Fmoc-protected amine was subjected to deprotection conditions resulting in Fmoc deprotection without observable conversion of the phosphotriester into a phosphodiester.

### Tccatgacgttcctgacgtt

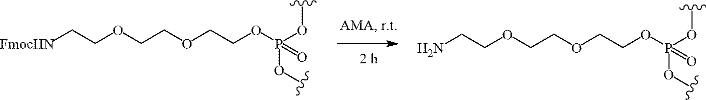

### DBCO-NHS Conjugation to TCCATGACGTTCCTGACGTT - Representative Example:

DBCO-NHS conjugation to the amino group in the phosphotriester was complete in 10 min at room temperature, as evidenced by mass spectrometric analysis.

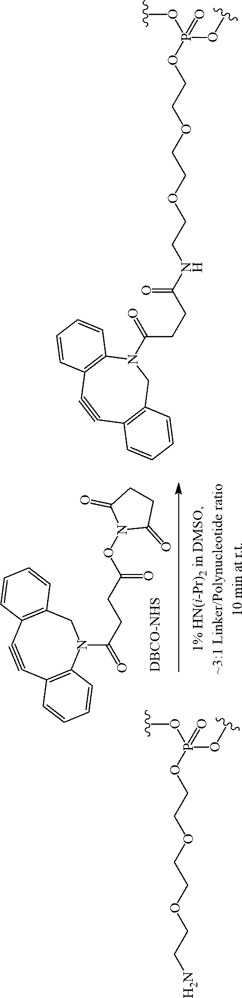

RP-HPLC purification of TCCATGACGTTCCTGACGTT containing a DBCO conjugating group was performed using the following conditions:
Buffer A = 50 mM TEAA in Water;
Buffer B = 90% Acetontrile; and
Flow Rate = 1 mL/min;
Gradient:
   0 - 2 min (100% Buffer A / 0% Buffer B),
   2 - 22 min (0% to 100% Buffer B), and
   22 - 25 min (100% Buffer B).

A similar procedure may be used to prepare a polynucleotide using, e.g., 2′-modified nucleoside phosphoramidites, such as those described herein. Such a procedure is provided in International Patent application PCT/US2015/034749; the disclosure of the disulfide phosphotriester oligonucleotide synthesis in PCT/US2015/034749 is hereby incorporated by reference.

### V. Intermediate Compounds

Also provided herein are intermediates compounds, such as the compounds of Formula (I), Formula (II), and Formula (III), that can be used to prepare a conjugate of Formula (A), (A1), (A2), (B) or (B1) via methods described herein.

In one aspect, provided herein are compounds of Formula (I):

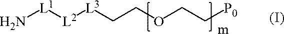

or a stereoisomer, a mixture of two or more diastereomers, a tautomer, or a mixture of two or more tautomers thereof; or a pharmaceutically acceptable salt, solvate, or hydrate thereof, wherein L¹, L², L³, m and P₀ are as defined herein. In some embodiments, provided herein are compounds of Formula (I-a):

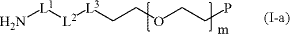

or a stereoisomer, a mixture of two or more diastereomers, a tautomer, or a mixture of two or more tautomers thereof; or a pharmaceutically acceptable salt, solvate, or hydrate thereof. wherein:
each L¹ is independently unsubstituted or substituted alkyl,
each L² is unsubstituted or substituted aryl, or unsubstituted or substituted heteroaryl,
each L³ is independently absent or a linker moiety,
m is an integer selected from the group consisting of 0-50, and
P is an immunomodulating oligonucleotide.

In another aspect, provided herein re compounds of Formula (II):

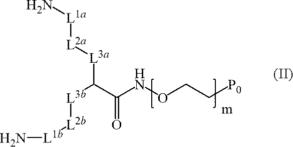

or a stereoisomer, a mixture of two or more diastereomers, a tautomer, or a mixture of two or more tautomers thereof; or a pharmaceutically acceptable salt, solvate, or hydrate thereof, wherein L^{1a}, L^{2a}, L^{3a}, L^{1b}, L^{2b}, L^{3b}, m and P₀ are as defined herein. In some embodiments, provided herein are compounds of formula (II-a):

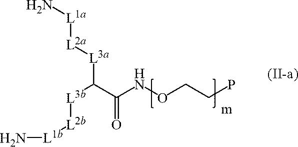

or a stereoisomer, a mixture of two or more diastereomers, a tautomer, or a mixture of two or more tautomers thereof; or a pharmaceutically acceptable salt, solvate, or hydrate thereof; wherein:
L^{1a} and L^{1b} are independently unsubstituted or substituted alkyl,
L^{2a} and L^{2b} are independently absent, unsubstituted or substituted alkyl, unsubstituted or substituted aryl, or unsubstituted or substituted heteroaryl,
L^{3a} and L^{3b} are independently absent or a linker moiety,
m is an integer selected from the group consisting of 0-50, and
P is an immunomodulating oligonucleotide.

In yet another aspect, provided herein are compounds of Formula (III):

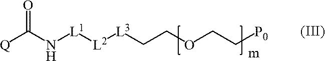

or a stereoisomer, a mixture of two or more diastereomers, a tautomer, or a mixture of two or more tautomers thereof; or a pharmaceutically acceptable salt, solvate, or hydrate thereof,, wherein L¹, L², L³, m and P₀ are as defined herein. In some embodiments, provided herein are compounds of Formula (III-a):

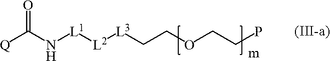

or a stereoisomer, a mixture of two or more diastereomers, a tautomer, or a mixture of two or more tautomers thereof; or a pharmaceutically acceptable salt, solvate, or hydrate thereof; wherein:
Q is a glutamine residue, wherein the glutamine residue is part of a Q-tag peptide sequence;
L¹ is unsubstituted or substituted alkyl,
L² is unsubstituted or substituted aryl, or unsubstituted or substituted heteroaryl,
L³ is absent or a linker moiety,
m is an integer selected from the group consisting of 0-50, and
P is an immunomodulating oligonucleotide.

In some embodiments, P is

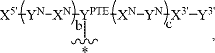

wherein
b and c are each independently an integer from 1 to 25; with the proviso that the sum of b and c is at least 5;
〰 * indicates the point of attachment of the immunomodulating oligonucleotide P to the rest of the conjugate;
X^{5′} is a 5′ terminal nucleoside having the structure

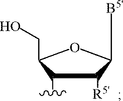

X^{3′} is a 3′ terminal nucleoside having the structure

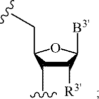

Y^{PTE} is an internucleoside phosphotriester having the structure

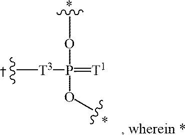

indicates the points of attachment to the rest of the oligonucleotide and † indicates the point of attachment to the rest of the conjugate;
Y^{3′} is a terminal phosphotriester having the structure

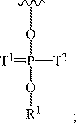

each X^{N} is independently a nucleoside having the structure

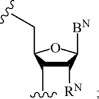

each Y^{N} is independently an internucleoside linker having the structure

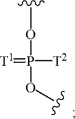

wherein each B^{N} is independently a modified or unmodified nucleobase;
each R^{N} is independently —H or —O—C₁-₄-alkyl, wherein the C₁-₄-alkyl of the —O—C₁—₄-alkyl is further optionally substituted by —O—C₁-C₄-alkyl;
B^{5′}and B^{3′} are independently a modified or unmodified nucleobase;
R^{5′} and R^{3′} are independently —H or —O—C₁-C₄-alkyl, wherein the C₁-₄-alkyl of the —O—C₁-₄-alkyl is further optionally substituted by —O—C₁-C₄-alkyl;
each T₁ is independently O or S;
each T₂ is independently O⁻ or S⁻; and
T₃ is a group comprising an oligoethylene glycol moiety; and
R¹ is C₁-₄-alkylene-hydroxy.

In some embodiments, (i) P comprises at least one modified nucleoside X^{N}; (ii) P has at least one modified internucleoside linker Y^{N}, wherein at least one of T¹ or T² is S; or (iii) both (i) and (ii). In some embodiments, P has at least one phosphorodithioate or phosphorothioate internucleoside linker. In certain embodiments, P comprises 0, 1, 2 or 3 phosphorodithioate internucleoside linkers. In some embodiments, P comprises one or more CpG sites. In some embodiments, the modified nucleotide is selected from the group consisting of 2′-O-alkyl nucleotide, 2′-O-alkoxyalkyl nucleotide, 2′ -deoxy nucleotide and ribonucleotide. In some embodiments, the modified nucleotide is selected from the group consisting of 5-bromo-2′-O-methyluridine, 5-bromo-2′-O-methyl-deoxyuridine, 5-bromo-2′-deoxyuridine, 2′-O-methylthymidine, 2′-O-methylcytidine, 2′-O-(2-methoxyethyl)thymidine and 8-oxo-7,8-dihydro-2′-deoxyguanosine. In yet other embodiments, Y³’ or the Y^{N} at the 3′ position of X^{5′} comprises an unsubstituted or substituted phosphorothioate. In some embodiments, P comprises an oligonucleotide sequence selected from the group consisting of SEQ ID NOS: 1-5. In some embodiments, m is 15-30. In some embodiments, e is 1 or 2. In some embodiments, Y^{PTE} is:

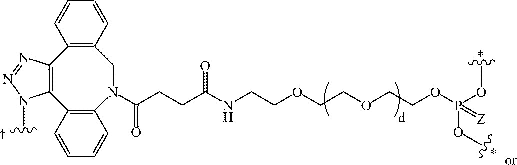

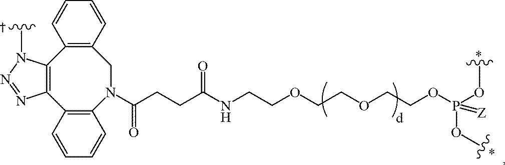

wherein Z is O or S; d is an integer from 0 to 95; the two 〰 * on the right side of the structure indicate the points of attachment to the adjacent nucleosides X^{N} in the oligonucleotide P, and the one † on the left side of the structure indicates the point of attachment to the rest of the conjugate. In some embodiments, Y^{PTE} is:

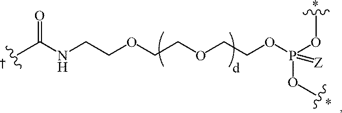

wherein Z is O or S; d is an integer from 0 to 95; the two 〰 * on the right side of the structure indicate the points of attachment to the adjacent nucleosides X^{N} in the oligonucleotide P, and the † on the left side of the structure indicates the point of attachment to the rest of the conjugate . In some embodiments, Z is S.

In some embodiments, L¹ is methyl or ethyl. In some embodiments, L¹ is substituted by an unsubstituted or substituted aryl. In some embodiments, L¹ is substituted by phenyl. In some embodiments, L² is a 6-10 membered aryl. In some embodiments, L² is phenyl. In some embodiments, L² is a 6-10 membered heteroaryl. In some embodiments, L² is pyridinyl. In some embodiments, L³ is a linker moiety. In some embodiments, the linker moiety is an unsubstituted or substituted alkyl. In some embodiments, the linker is methyl. In some embodiments, the linker moiety is —R¹C(O)R²NHR³—, wherein R¹ and R³ are independently absent or unsubstituted or substituted alkyl and R² is an amino acid residue. In some embodiments, the amino acid is selected from the group consisting of glycine, alanine, glutamic acid and proline. In some embodiments, the linker moiety is —R⁴C(O)NHR⁵— or —R⁴NHC(O)R⁵—, wherein R⁴ and R⁵ are independently absent or unsubstituted or substituted alkyl. In some embodiments, R⁴ is methylene and R⁵ is —(CH₂)₄—. In some embodiments, R⁴ is methylene and R⁵ is absent.

In some embodiments, Q is a glutamine residue, wherein the glutamine residue is part of a Q-tag peptide sequence. In some embodiments, the Q-tag comprises a peptide selected from the group consisting of SEQ ID NOS: 6-18.

### VI. Pharmaceutical Compositions

The compounds and conjugates of the present invention, such as the conjugates of Formula (A), (A1), (A2), (B), and (B1), or a pharmaceutically acceptable salt of any of the foregoing, or any subgroup thereof may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their ease in administration, tablets and capsules represent the most advantageous oral dosage unit forms, in which case solid pharmaceutical carriers are employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. The compounds of the present invention may also be administered via oral inhalation or insufflation in the form of a solution, a suspension or a dry powder using any art-known delivery system.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

Administration can be, but is not limited to, intravenous, intraarterial, subcutaneous, intraperitoneal, subdermal (e.g., via an implanted device), and intraparenchymal administration. In some embodiments, the pharmaceutical compositions described herein are administered by subcutaneous injection.

The pharmaceutical compositions including a conjugate described herein can be delivered to a cell, group of cells, tumor, tissue, or subject using delivery technologies known in the art. In general, any suitable method recognized in the art for delivering a nucleic acid-protein conjugate (in vitro or in vivo) can be adapted for use with a herein described compositions. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In certain embodiments, the compositions are administered by subcutaneous or intravenous infusion or injection.

Accordingly, in some embodiments, the herein described pharmaceutical compositions may comprise one or more pharmaceutically acceptable excipients. In some embodiments, the pharmaceutical compositions described herein can be formulated for administration to a subject.

As used herein, a pharmaceutical composition or medicament includes a pharmacologically effective amount of at least one of the described therapeutic compounds or conjugates and one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, NJ) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The compound or conjugate can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.).

Generally, an effective amount of an active compound will be in the range of from about 0.1 to about 100 mg/kg of body weight/day, e.g., from about 1.0 to about 50 mg/kg of body weight/day. In some embodiments, an effective amount of an active compound will be in the range of from about 0.25 to about 5 mg/kg of body weight per dose. In some embodiments, an effective amount of an active compound will be in the range of 25-400 mg per 1-18 weeks or 1-6 months. In some embodiments, an effective amount of an active compound will be in the range of 50-125 mg per 4 weeks or per one month. In some embodiments, an effective amount of an active ingredient will be in the range of from about 0.5 to about 3 mg/kg of body weight per dose. In some embodiments, an effective amount of an active ingredient will be in the range of from about 25-400 mg per dose. In some embodiments, an effective amount of an active ingredient will be in the range of from about 50-125 mg per dose. The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level in order to rapidly achieve the desired blood-level or tissue level, or the initial dosage can be smaller than the optimum.

For treatment of disease or for formation of a medicament or composition for treatment of a disease, the pharmaceutical compositions described herein including a compound or conjugate can be combined with an excipient or with a second therapeutic agent or treatment including, but not limited to: a second or other conjugates, a small molecule drug, an antibody, an antibody fragment, and/or a vaccine.

The described compounds or conjugates, when added to pharmaceutically acceptable excipients or adjuvants, can be packaged into kits, containers, packs, or dispensers. The pharmaceutical compositions described herein may be packaged in pre-filled syringes or vials.

### VI. Kits

Also provided herein is a kit comprising a conjugate of Formula (A), (A1), (A2), (B), or (B1) as described above.

In another aspect, the kit further comprises a package insert including, without limitation, appropriate instructions for preparation and administration of the formulation, side effects of the formulation, and any other relevant information. The instructions may be in any suitable format, including, but not limited to, printed matter, videotape, computer readable disk, optical disc or directions to internet-based instructions.

In another aspect, kits for treating an individual who suffers from or is susceptible to the conditions described herein are provided, comprising a first container comprising a dosage amount of a composition or formulation as disclosed herein, and a package insert for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the individual.

In another aspect, kits may also be provided that contain sufficient dosages of the compositions described herein (including pharmaceutical compositions thereof) to provide effective treatment for an individual for an extended period, such as 1-3 days, 1-5 days, a week, 2 weeks, 3, weeks, 4 weeks, 6 weeks, 8 weeks, 1 cycle, 2 cycles, 3 cycles, 4 cycles, 5 cycles, 6 cycles, 7 cycles, 8 cycles or more.

In some embodiments, the kits may also include multiple doses and may be packaged in quantities sufficient for storage and use in pharmacies, for example, hospital pharmacies and compounding pharmacies. In certain embodiments the kits may include a dosage amount of at least one composition as disclosed herein.

In another aspect, kits may also be provided that contain a conjugate of Formula (A), (A1), (A2), (B), or (B1).

### VII. Methods of Treatments

Also provided herein are methods for treating a disease or disorder in a subject comprising administering an effective amount of a compound or conjugate described herein to the subject in need thereof. Also provided herein are uses of a compound or conjugate described herein in the preparation of a medicament for treating a patient in need of treatment with the oligonucleotide in the conjugate. Also provided are compounds or conjugates as described herein for treating a disease or disorder in a subject in need of the treatment with the oligonucleotide in the compounds or conjugates. Also provided are compounds or conjugates as described herein for treating a patient comprising administering an effective amount of the compound or conjugate to the patient. In some embodiments, the subject has or at the risk of developing cancer. In some embodiments, the disease or disorder is a viral infection. In some embodiments, the disease or disorder is an immunodeficiency, e.g., in which immune activation may be favorable. In some embodiments, the disease or disorder is an autoimmune and/or inflammatory disease or disorder, e.g., in which immune suppression and/or modulation may be favorable.

In some embodiments of the methods of treating cancer as described herein, the cancer being treated with the methods disclosed herein is a solid tumor. In some embodiments, the cancer being treated with the methods disclosed herein is a liquid tumor. In some embodiments, the cancer being treated with the methods disclosed herein is a solid tumor. In particular embodiments, the cancer being treated with the methods disclosed herein is breast cancer, colorectal cancer, lung cancer, head and neck cancer, melanoma, lymphoma or leukemia. In some embodiments, cancers include, but are not limited to, B cell cancer, e.g., multiple myeloma, Waldenström’s macroglobulinemia, the heavy chain diseases, such as, for example, alpha chain disease, gamma chain disease, and mu chain disease, benign monoclonal qammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, sominoma, embryonal carcinoma, Wilms’ tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin’s disease and non-Hodgkin’s disease), multiple myeloma, Waldenstrom’s macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated. In particular embodiments, the cancer being treated with the methods disclosed herein is selected from the list consisting of mantle cell cymphoma (MCL), diffuse large B-cell lymphoma (DLBCL), Burkitts lymphoma, multiple melanoma (MM), chronic lymphocytic leukemia (CLL), acute myeloid leukemia (AML), small lymphocytic lymphoma (SLL), hairy cell leukemia (HCL), lymphoplasmacytic lymphoma (LPL), skeletal muscle lymphoma (SML), splenic marginal zone lymphoma (SMZL), follicle center lymphoma (FCL), colorectal cancer, non-small cell lung cancer (NSCLC), head and neck cancer, breast cancer, pancreatic cancer, glioblastoma (GBM), prostate cancer, esophageal cancer, renal cell carcinoma, hepatic carcinoma, bladder cancer and gastric carcinoma.

In some embodiments, the cancer being treated with the methods disclosed herein is resistant to at least one immunotherapy. In some embodiments, the cancer being treated with the methods disclosed herein is resistant to at least one cancer therapy selected from the group consisting of chemotherapy, radiation, targeted therapy, vaccine therapy, and CAR-T therapy. In some embodiments, the method of treating cancer comprises co-administering to a subject having cancer (i) a therapeutically effective amount of the CpG-containing immunostimulating polynucleotide or the CpG-antibody immunoconjugate; and (ii) the immunotherapeutic agent which the cancer being treated has shown to resist or not to respond, when the cancer is treated with the immunotherapeutic agent alone.

In particular embodiments, the cancer being treated with the methods provided herein has been shown to not to respond to a treatment with an immune checkpoint modulator. In particular embodiments, the immune checkpoint modulator is an inhibitor of PD-1. In particular embodiments, the immune checkpoint modulator is an inhibitor of PD-L1. In some embodiments, the method of treating cancer comprises co-administering to a subject having cancer (i) a therapeutically effective amount of the CpG-containing immunostimulating polynucleotide or the CpG-Ab immunoconjugate; and (ii) a therapeutically effective amount of the inhibitor of PD-1. In some embodiments, the method of treating cancer comprises co-administering to a subject having cancer (i) a therapeutically effective amount of the CpG-containing immunostimulating polynucleotide or the CpG-Ab immunoconjugate; and (ii) a therapeutically effective amount of the inhibitor of PD-L1. In particular, in some embodiments, the inhibitor of PD-1 is an anti-PD-1 antibody or an antigen-binding fragment thereof. In some embodiments, the inhibitor of PD-L1 is an anti-PD-L1 antibody or an antigen-binding fragment thereof.

In certain aspects, provided herein are methods of preventing cancer in a subject susceptible of developing cancer, comprising administering to the subject a therapeutically effective amount of a TLR agonist as described herein. In some embodiments, the method comprising administering to the subject a therapeutically effective amount of a CpG-containing immunostimulating polynucleotide or a CpG-Ab immunoconjugate described herein. In particular embodiments, the CpG-Ab immunoconjugate targets a normal immune cell as described herein. In particular embodiments, the CpG-Ab immunoconjugate targets a TLR-expressing cell as described herein. In particular embodiments, the CpG-Ab immunoconjugate specifically binds to an antigen associated with a normal immune cell as described herein. In particular embodiments, the CpG-Ab immunoconjugate specifically binds to an antigen associated with a normal immune cell does not bind to a tumor-associated antigen of the cancer being prevented. In particular embodiments, the CpG-Ab immunoconjugate specifically binds to an antigen associated with a TLR-expressing cell as described herein. In particular embodiments, the CpG-Ab immunoconjugate specifically binds to an antigen associated with a TLR-expressing cell and does not bind to a tumor-associated antigen of the cancer being prevented. In particular embodiments, the CpG-Ab immunoconjugate specifically binds to a tumor-associated antigen of the cancer being prevented as described herein, e.g., an antigen expressed on a tumor cell surface. In particular embodiments, a tumor-associated antigen of the cancer being prevented is also associated with a normal immune cell or a TLR-expressing cell. In particular embodiments, the CpG-Ab immunoconjugate does not specifically bind to an antigen selected from CD19, CD20, CD22, STAT3, exportin 7, Her2, Src, EGFR, CD52, CXCR-4, and Muc-1.

In some embodiments, the methods of preventing cancer further comprises administering to a subject susceptible to developing cancer (i) a therapeutically effective amount of a CpG-Ab immunoconjugate and (ii) a tumor-associated antigen of the cancer being prevented. In some embodiments, the tumor-associated antigen is not conjugated to the CpG-Ab immunoconjugate. In particular embodiments, the tumor-associated antigen is formulated as a cancer vaccine. In particular embodiments, the CpG-Ab immunoconjugate is formulated as an adjuvant of the cancer vaccine.

In some embodiments, the cancer being prevented or treated using the methods provided herein is an episode of cancer recurrence in a subject who is in partial or complete remission of a prior cancer. In particular embodiments, the prior cancer is a liquid cancer and the recurrent cancer being prevented or treated is a liquid cancer. In particular embodiments, the prior cancer is a solid cancer and the recurrent cancer being prevented or treated is a solid cancer. In particular embodiments, the prior cancer is a liquid cancer and the recurrent cancer being prevented or treated is a solid cancer. In particular embodiments, the prior cancer is a solid cancer and the recurrent cancer being prevented or treated is a liquid cancer.

In some embodiments, the cancer being prevented or treated using the methods provided herein is first episode of cancer recurrence in the subject after the subject showed partial or complete remission. In some embodiments, the cancer being prevented or treated using the methods provided herein is second episode of cancer recurrence in the subject after the subject showed partial or complete remission. In some embodiments, the cancer being prevented or treated using the methods provided herein is third episode of cancer recurrence in the subject after the subject showed partial or complete remission. In some embodiments, the cancer being prevented or treated using the methods provided herein is an episode of cancer recurrence subsequent to the third episode of cancer recurrence in the subject after the subject showed partial or complete remission.

In certain aspects, provided herein are methods of inducing an adaptive immune response in a subject in need thereof, wherein method comprises administering to the subject a therapeutically effective amount of a TLR agonist as described herein. In particular embodiments, the method of inducing an adaptive immune response comprises administering to the subject in need thereof a therapeutically effective amount of a CpG-containing immunostimulating polynucleotide or a CpG-Ab immunoconjugate described herein. In particular embodiments, the CpG-Ab immunoconjugate targets a normal immune cell as described herein. In particular embodiments, the CpG-Ab immunoconjugate targets a TLR-expressing cell as described herein. In particular embodiments, the CpG-Ab immunoconjugate targets a diseased cell selected from a cancer cell or a pathogen infected cell. In particular embodiments, the CpG-Ab immunoconjugate specifically binds to an antigen associated with a normal immune cell as described herein. In particular embodiments, the CpG-Ab immunoconjugate specifically binds to an antigen associated with a normal immune cell does not bind to a disease antigen. In particular embodiments, the CpG-Ab immunoconjugate specifically binds to an antigen associated with a TLR-expressing cell as described herein. In particular embodiments, the CpG-Ab immunoconjugate specifically binds to an antigen associated with a TLR-expressing cell does not bind to a disease antigen. In particular embodiments, the CpG-Ab immunoconjugate specifically binds to a disease antigen as described herein. In particular embodiments, the diseased antigen is also associated with a normal immune cell or a TLR-expressing cell. In particular embodiments, the diseased antigen is a tumor-associated antigen or a pathogenic antigen. In particular embodiments, the CpG-Ab immunoconjugate does not specifically bind to an antigen selected from CD19, CD20, CD22, STAT3, exportin 7, Her2, Src, EGFR, CD52, CXCR-4, and Muc-1.

In some embodiments of the methods and uses described herein, the CpG-containing immunostimulating polynucleotide is administered to a subject in need thereof at a dosage that is sufficient for activating the TLR9-mediated signaling pathway in the subject. In some embodiments, the CpG-Ab immunoconjugate is administered to a subject in need thereof at a dosage that is sufficient for activating the TLR9 mediated signaling pathway in a cell population targeted by the CpG-Ab immunoconjugate. As described herein, in some embodiments, the cell population targeted by the CpG-Ab immunoconjugate expresses TLR9. In some embodiments, the cell population targeted by the CpG-Ab immunoconjugate can express the TLR9 on the cell surface of the targeted cell, on the endosomal membrane of the targeted cell, or both on the cell surface and on the endosomal membrane of the targeted cell.

Particularly, in some embodiments of the methods and uses described herein, the CpG-containing immunostimulating polynucleotide is administered to a subject in need thereof at a dosage that is effective for inducing one or more of effects selected from (a) specifically binding to a TLR9 receptor by the CpG-containing immunostimulating polynucleotide on a targeted cell; (b) efficient internalization of the CpG-Ab immunoconjugate or the CpG-containing immunostimulating polynucleotide portion thereof by a targeted cell; (c) activating one or more signaling pathways in the targeted cell; (d) inducing secretion of one or more inflammatory cytokines by the targeted cell; (e) suppressing secretion of one or more inflammatory cytokines by the targeted cell; (f) upregulating expression of one or more genes of the targeted cell; (g) suppressing expression of one or more genes of the targeted cell; (h) activating targeted normal immune cells, (i) inducing an immune response that results in the elimination of disease, e.g., cancer cells, including (j) apoptosis of a targeted cancer cell, and (k) inducing necrosis of targeted cancer cell.

Particularly, in some embodiments of the methods and uses described herein, wherein upon administration of the CpG-Ab immunoconjugate, the CpG-containing immunostimulating polynucleotide specifically binds to a TLR9 receptor of the targeted cell. Particularly, in some embodiments, binding of CpG-Ab immunoconjugate to an antigen associated with a targeted cell facilitates specific binding of the CpG-containing immunostimulating polynucleotide to a TLR9 receptor. In some embodiments, the target antigen of the CpG-Ab immunoconjugate is located near the TLR9 receptor. In particular embodiments, both the target antigen and the TLR9 receptor locate on the cell membrane of the targeted cell. In particular embodiments, both the target antigen and the TLR9 receptor locate on an intracellular membrane of the targeted cell. In particular embodiments, both the target antigen and the TLR9 receptor locate on the endosomal or phagosomal membrane of the targeted cell. In some embodiments, the target antigen locates on the cell membrane and facilitates internalization of the CpG-Ab immunoconjugate into the cytosol upon binding to the CpG-Ab immunoconjugate.

Particularly, in some embodiments of the methods and uses described herein, the method comprises administering to a subject in need thereof a therapeutically effective amount of a CpG-Ab immunoconjugate targeting a normal immune cell, wherein upon administration of the CpG-Ab immunoconjugate, one or more immunogenic signaling pathways in the targeted cell are activated. In particular embodiments, the activated signaling pathways are one or more selected from the nuclear factor (NF)- κB signaling pathway, the c-Jun N-terminal kinase (JNK) signaling pathway, the AP1 signaling pathway, the IRF3/7 pathway, and the p38 mitogen-activated protein kinase (MAPK) signaling pathway. The activation of a cellular signaling pathway can be detected using methods known in the art, such as but not limited to, detecting the presence of a molecular marker of which the expression is specifically induced upon activation of the signaling pathway of interest.

Particularly, in some embodiments of the methods and uses described herein, the method comprises administering to a subject in need thereof a therapeutically effective amount of a CpG-Ab immunoconjugate targeting a normal immune cell, wherein upon administration of the CpG-Ab immunoconjugate, secretion of one or more inflammatory cytokines is induced. In particular embodiments, the one or more inflammatory cytokines are selected from type I interferon (IFN), interleukin (IL)-6, IL10, IL-12, IL-18, and tumor necrosis factor (TNF).

Particularly, in some embodiments of the methods and uses described herein, the method comprises administering to a subject in need thereof a therapeutically effective amount of a CpG-Ab immunoconjugate targeting a normal immune cell, wherein upon administration of the CpG-Ab immunoconjugate, expression of one or more additional proteins are upregulated. In particular embodiments, the upregulated proteins are one or more selected from antigen presenting molecules (e.g., MHC class I and II), cytokine receptors (e.g., IL-6 receptors, IL-10 receptors, IL-12 receptors, TNF-α receptor, TNF-β receptor, IFN-α receptor, IFN-β receptor, IFN-γ), chemokine receptors (e.g., chemokine receptor 7), costimulatory molecules (e.g., CD3, CD28, CD27, CD30, CD40, CD69, CD80/B7-1, CD86/B7-2, CD134/OX-40, OX-40L, CD137/4-1BB, 4-1BBL, CD278/ICOS, B7-H3, B7h/B7RP-1, LIGHT etc.), HLA-DR and T cell maturation regulatory proteins (e.g., indoleamine 2,3-dioxygenase).

Particularly, in some embodiments of the methods and uses described herein, the method comprises administering to a subject in need thereof a therapeutically effective amount of a CpG-Ab immunoconjugate targeting a normal immune cell, wherein upon administration of the CpG-Ab immunoconjugate, proliferation, differentiation, maturation and/or survival of one or more populations of normal immune cells are increased. In particular embodiments, the one or more increased populations of normal immune cells are selected from CD4+ T cells, CD8+ T cells, natural killer cells, T helper cells, B cells, and myeloid cells (including mDCs and pDCs). in some embodiments of the methods and uses described herein, the method comprises administering to a subject in need thereof a therapeutically effective amount of a CpG-Ab immunoconjugate targeting a normal immune cell, wherein upon administration of the CpG-Ab immunoconjugate, proliferation, differentiation, maturation and/or survival of one or more populations of normal immune cells are reduced. In particular embodiments, the one or more reduced populations of normal immune cells is selected from B-reg cells, T-reg cells, and MDSCs.

In particular embodiments, upon administration of the CpG-Ab immunoconjugate, antigen presentation activities are increased in APCs in the subject. In some embodiments, the APC is selected from B cells, monocytes, dendritic cells, and Langerhans cells, keratinocytes, endothelial cells, astrocytes, fibroblasts, and oligodendrocytes. In particular embodiments, the APC is B cells. In particular embodiments, the APC is dendritic cells. In particular embodiments, the APC is macrophage. In some embodiments, the dendritic cell is pDC. In particular embodiments, the increased antigen presentation activities lead to more efficient presentation of a tumor-associated antigen by the activated APCs.

In particular embodiments, upon administration of the CpG-Ab immunoconjugate, antigen-specific CD4+ T cell mediated immunity against one or more tumor-associated antigen of the cancer being treated or prevented is increased. In particular embodiments, upon administration of the CpG-Ab immunoconjugate, tumor infiltration by CD4+ T cell is increased. In particular embodiments, upon administration of the CpG-Ab immunoconjugate, antigen-specific CD8+ T cell mediated immunity against one or more tumor-associated antigen of the cancer being treated or prevented is increased is increased. In particular embodiments, upon administration of the CpG-Ab immunoconjugate, tumor infiltration by CD8+ T cell is increased. In particular embodiments, upon administration of the CpG-Ab immunoconjugate, B cell secretion of immunoglobulin specifically against one or more tumor-associated antigen of the cancer being treated or prevented is increased is increased.

Particularly, in some embodiments of the methods and uses described herein, the method comprises administering to a subject in need thereof, a therapeutically effective amount of a CpG-Ab immunoconjugate targeting a diseased cell, wherein upon administration of the CpG-Ab immunoconjugate, one or more apoptotic signaling pathways are induced trigger apoptosis of the targeted diseased cell. In some embodiments, the diseased cell is a cancer cell.

In some embodiments of the methods and uses described herein, the CpG-Ab immunoconjugate is administered to a subject in need thereof in an amount that is not effective for activating the complement system in the subject. In some embodiments, the CpG-containing immunostimulating polynucleotide is administered to a subject in need thereof in an amount that is not effective to activate complement C1 in the subject. In some embodiments, the CpG-containing immunostimulating polynucleotide is administered to a subject in need thereof in an amount that is not effective to activate complement C3 in the subject. Complement activation can be detected using methods known in the art. In some embodiments, the CpG-Ab immunoconjugate is administered to a subject in need thereof in an amount that is not effective for the antibody portion of the CpG-Ab immunoconjugate to induce antibody-dependent cell-mediated cytotoxicity in the subject.

As described herein, therapeutic agents, conjugates or compositions comprising the CpG-containing polynucleotides can be used in combination with at least one additional therapeutic agent for preventing or treating cancer. In some embodiments, such combination therapy exhibits a synergistic therapeutic effect that is better than the separate effect of either therapeutic agent alone. In some embodiments, such combination therapies exhibit a synergistic therapeutic effect that is better than the sum of the separate effects of the therapeutic agents alone.

Accordingly, in certain aspects, provided herein are methods for preventing or treating cancer using the CpG-containing immunostimulating polynucleotide in combination with at least one additional cancer therapeutic agent. Such methods comprising administering to a subject in need thereof (i) a therapeutically effective amount of the CpG-containing immunostimulating polynucleotide, and (ii) a therapeutically effective amount of at least one additional cancer therapeutic agents. In particular embodiments, the CpG-containing immunostimulating polynucleotide is administered as a free-standing polynucleotide. In particular embodiments, the CpG-containing immunostimulating polynucleotide is administered as a CpG-Ab immunoconjugate. In particular embodiments, the CpG-containing immunostimulating polynucleotide and the additional therapeutic agents are formulated in the same composition. In other embodiments, CpG-containing immunostimulating polynucleotide and the additional therapeutic agents are formulated in the separate compositions.

In some embodiments, the at least one additional cancer therapeutic agent is selected from T cell agonists, immune checkpoint modulators, STING agonists, RIG-I agonists, other toll-like receptor agonists.

In some embodiments, the additional cancer therapeutic agent is a T cell costimulatory molecule. In some embodiments, the T cell costimulatory molecule is selected from OX40, CD2, CD27, CDS, ICAM-1, LFA-1/CD11a/CD18, ICOS/CD278, 4-1BB/CD137, GITR, CD30, CD40, BAFFR, HVEM, CD7, LIGHT, NKG2C, SLAMF7, NKp80, CD160, B7-H3, and CD83, or a ligand thereof. In some embodiments, a ligand of a costimulatory molecule is an antibody specifically binding to the costimulatory molecule. In particular embodiments, the additional cancer therapeutic agent is selected from an anti-OX40 antibody, an anti-OX40L antibody, an anti-ICOS antibody, an anti-CTLA4 antibody, an anti-CD40L antibody, an anti-CD28 antibody, an anti-LFA1 antibody, an anti-TIM1/TIM3 antibody, anti-LAG3 antibody, anti-Siglec-15 antibody, an anti-PD1 antibody, an anti-PDL1 antibody, an anti-CD27 antibody and an anti-4-1BB antibody.

In some embodiments, the additional cancer therapeutic agent is a tumor-associated antigen produced by the cancer that is being prevented or treated with the method. In some embodiments, the cancer being prevented or treated is leukemia, lymphoma, melanoma, colorectal, breast, prostate, renal, pancreatic, head and neck, skin, and brain cancer, lung cancer, and the tumor-associated antigen is selected from CD19, CD20, CD22, CD38, CD138, CD30, CD52, CD56, CD79, CD123, CD206, CD303, CD304, EGFR, folate receptor alpha, folate receptor beta, mesothelin, Her2, transferrin receptor, and PSMA. In some embodiments, the additional cancer therapeutic agent is an immune checkpoint modulator selected from inhibitors of PD-1, PD-L1, PD-L2, TIM-3, LAG-3, CEACAM-1, CEACAM-5, CLTA-4, VISTA, BTLA, TIGIT, LAIR1, CD47, SIRP-α, CD160, 2B4, CD172a, and TGFR. In particular embodiments, the additional cancer therapeutic agent is a PD-1 inhibitor. In particular embodiments, the additional cancer therapeutic agent is a PD-L1 inhibitor. In particular embodiments, the additional cancer therapeutic agent is a CD47 inhibitor. In some embodiments, the additional cancer therapeutic agent is an antibody specifically binding to the immune checkpoint modulator. In particular embodiments, the additional cancer therapeutic agent is an anti-PD-1 antibody or an antigen-binding fragment thereof. In particular embodiments, the additional cancer therapeutic agent is an anti-PD-L1 antibody or an antigen-binding fragment thereof. In particular embodiments, the additional cancer therapeutic agent is an anti-CD47 antibody or an antigen-binding fragment thereof. In particular embodiments, the additional cancer therapeutic agent is an anti-CD172a antibody or an antigen-binding fragment thereof, In particular embodiments, the additional cancer therapeutic agent is an anti-OX40 antibody or an antigen-binding fragment thereof, In particular embodiments, the additional cancer therapeutic agent is an anti-TIM3 antibody or an antigen-binding fragment thereof, In particular embodiments, the additional cancer therapeutic agent is an anti-LAG3 antibody or an antigen-binding fragment thereof. Anti-PD-1 and anti-PD-L1 antibodies and their uses are described in, for example, US20180030137, US9815898, US20170313776, US20170313774, US20170267762, WO2017019846, WO2018013017, US20180022809, US20180002423, WO2017220990, WO2017218435, WO2017215590, US9828434, and WO2017196867. Anti-CD47 antibodies and their uses are described in, for example US9663575, US9803016, US20170283498, US20170369572, WO2017215585, WO2017196793, and WO2017049251.

In some embodiments, the additional cancer therapeutic agent is a STING pathway agonist. STING (stimulator of interferon genes, also known as TMEM173, MITA, ERIS, and MPYS) is a transmembrane protein localized to the ER that undergoes a conformational change in response to direct binding of cyclic dinucleotides (CDNs), resulting in a downstream signaling cascade involving TBK1 activation, IRF-3 phosphorylation, and production of IFN-β and other cytokines. The STING pathway in tumor-resident host antigen presenting cells is involved in the induction of a spontaneous CD8+ T cell response against tumor-associated antigens. Activation of this pathway and the subsequent production of IFN-β also contributes to the anti-tumor effect. In some embodiments, the STING pathway agonist is ADU-S100. Additional STING agonists and their uses are described in, for example, US20180028553, US20170319680, US20170298139, US20060040887, US20080286296, US20120041057, US20140205653, WO2014179335, WO 2014179760, US20150056224, WO 2016096174, WO 2017011444, WO 2017027645, and WO 2017027646.

In some embodiments, the additional cancer therapeutic agent is a RIG-I pathway agonist. RIG-I (retinoic acid-inducible gene-I) is a member of pattern-recognition receptors that initiates a host’s innate immune system to defend against pathogenic microbes in early phases of infection. There are three members of the (RIG-I)-like receptors family: RIG-I, MDA5 (melanoma differentiation factor 5), and LGP2 (laboratory of genetics and physiology 2), which are expressed in most cell and tissue types. RIG-I functions as a cytoplasmic sensor for the recognition of a variety of RNA viruses and subsequent activation of downstream signaling to drive type I IFN production and antiviral gene expressions. Activated RIG-I recruits its downstream adaptor molecule MAVS (also known as IPS-1, CARDIF, and VISA) through CARD-CARD-mediated interactions. The oligomeric RIG-I CARD assembly and the polymeric formation of MAVS, together serve as a signaling platform for protein complexes that mediate the bifurcation of signaling into two branches. One branch recruits tumor necrosis factor receptor-associated factors (TRAF)-2/6 and the receptor-interacting protein 1 to subsequently activate the IKK complex, resulting in NF-κB activation. The other branch signals through TRAF3 and activates the TANK/IKKγ/IKK∈/TBK1 complex, leading to the phosphorylation and dimerization of interferon regulator factors (IRF)-3 and -7. Liu et al., Front Immunol. 2017, 7:662. Activation of this pathway contributes to the anti-tumor effect. In some embodiments, the RIG-I pathway agonist is RGT100. RIG-I agonists and their uses are described in, for example, US20170057978, US20170258897, US9381208, US9738680, US9650427, WO2017173427, and WO2017011622.

In some embodiments, the additional cancer therapeutic agent is a toll-like receptor agonist selected from TLR1 agonist, TLR2 agonist, TLR3 agonist, TLR4 agonist, TLR5 agonist, TLR6 agonist, TLR7 agonist, TLR8 agonist, and TLR10 agonist.

In further embodiments, in relation to a method of treating cancer, the CpG-containing immunostimulating polynucleotide is administered (either in the free-standing form or as a CpG-Ab immunoconjugate) in combination with one or more additional therapeutic agents or procedures, for example wherein the additional therapeutic agent or procedure is selected from the group consisting of chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule, a vaccine, a cellular immunotherapy, a cell-based therapy (e.g., CAR-T, TILs, TCR-T, CAR-NK, and CAR-macrophage therapies) and an oncolytic virus therapy.

### EXAMPLES

The following examples are offered to illustrate but not to limit the invention. One of skill in the art will recognize that the following procedures may be modified using methods known to one of ordinary skill in the art.

### Materials

Prototype peptides were made in house, but can be purchased at custom peptide suppliers (e.g., CPC Scientific). Oligonucleotides were made in-house or by LGC. Transglutaminase used in these examples were isolated from various bacterial Streptomyces strain (e.g., Ajinomoto). The Q-tag mAbs were produced at Sino Biologicals or internally.

### Production of Oligonucleotides

Oligonucleotides were generally prepared in accordance with the solid phase synthesis scheme shown below, beginning with an initial deprotection of the solid support for the oligonucleotide synthesis, followed by coupling of the solid support with to the first nucleotide, thiolation to give the phosphothioester and repeated deprotection and coupling to give the entire oligonucleotide sequence.

The general synthesis of oligonucleotides as provided herein is described below.

Deprotection: A dimethoxytrityl-1,3-propanediol glycolate protected controlled pore glass solid support (DMTO-C3-CPG, 1000 Å, Bulk Density 0.26-0.36 g/cc, Loading 30-40 µmol/g) was reacted with 3% dichloroacetic acid in toluene (v/v) at 25° C., to give the deprotected CPG support. UV absorption of an aliquot of the reaction mixture was measured to identify the reaction endpoint (wavelength 350 nm, target minimum absorbance 0.25 OD, using a fixed watch command setting) and to confirm removal of the dimethoxytrityl protecting group.

Activation/Coupling: The deprotected CPG support was coupled with the first nucleotide phosphoramidite precursor for the 3′-end, for the respective oligonucleotide to be synthesized, by adding and mixing the desired 3′ nucleotide (3 equiv.) for 5 minutes at 25° C. to the reactor containing the deprotected CPG support in the presence of an activator 5-Ethylthio-1H-tetrazole (0.5 M in ACN) at 60% of the nucleotide concentration.

Thiolation/Sulfurization: Following the coupling step, the linking phosphite triester moiety of the added nucleotide precursor was thiolated (or sulfurized) by adding Polyorg Sulfa (3-phenyl 1,2,4-dithiazoline-5-one), 0.15 M in dry ACN, to give the phosphothioester.

Capping: After sulfurization, the CPG support and linked nucleotide were treated with two capping compositions (Capping composition A: 20% N-methylimidazole in ACN; Capping B composition B: 20% Acetic Anhydride, 30% Pyridine, 50% ACN) to block unreacted nucleotide reactants.

Repeat Synthesis: The remaining nucleotides were added in sequence from the 3′ end to the 5′ end, employing the appropriate phosphoramidite precursors in solution, by repeating the steps of deprotection, activation/coupling, thiolation/sulfurization and capping as described above to obtain the desired oligonucleotide sequence in protected form. All phosphoramidite prescursors were mixed with the CPG support for 5 minutes during the coupling step, except for dT-Thiophosphoramidite, which was mixed for 15 minutes.

Selected phosphoramidite precursors used in the synthesis are shown below. The phosphoramidite precursors were prepared in solutions with the solvents and at the concentrations, respectively shown, to be used in the coupling steps.

| | | |
|---|---|---|
| Amidite | Structure | Concentration |
| DMT-dC(Ac) Amidite | 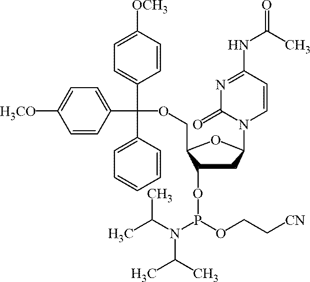 [SMILES: COc1ccc(C(OC[C@H]2O[C@@H](n3ccc(NC(C)=O)nc3=O)C[C@@H]2OP(OCCC#N)N(C(C)C)C(C)C)(c2ccccc2)c2ccc(OC)cc2)cc1] | 0.1 M in dry ACN: |
| DMT-dG(dmf) Amidite | 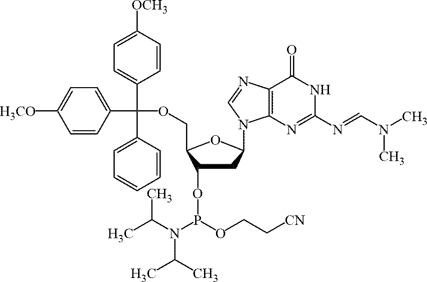 [SMILES: COc1ccc(C(OC[C@H]2O[C@@H](n3cnc4c(=O)[nH]c(/N=C/N(C)C)nc43)C[C@@H]2OP(OCCC#N)N(C(C)C)C(C)C)(c2ccccc2)c2ccc(OC)cc2)cc1] | 0.1 M in dry ACN: |
| DMT-dT phosphoramidite | 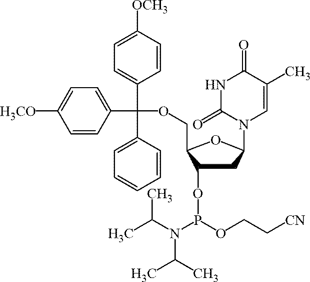 [SMILES: COc1ccc(C(OC[C@H]2O[C@@H](n3cc(C)c(=O)[nH]c3=O)C[C@@H]2OP(OCCC#N)N(C(C)C)C(C)C)(c2ccccc2)c2ccc(OC)cc2)cc1] | 0.1 M in dry ACN: |
| Fmoc-protected DMT- dT PEG2 NH2 Amidite | 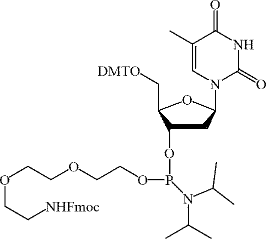 | 0.1 M in dry ACN |
| 5-Br-dU-CE Phosphoramidite | 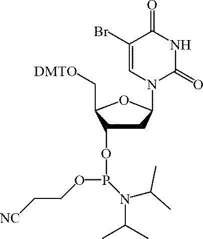 | 0.1 M in dry ACN |
| dT-Thiophsophoramidite | 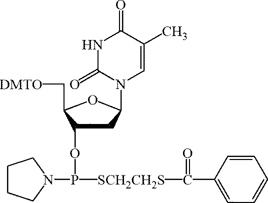 | 0.15 M in dry 10% (v/v) DCM/ACN |
| 2′-O-Methyl 5-Methyl Uridine CED phosphoramidite | 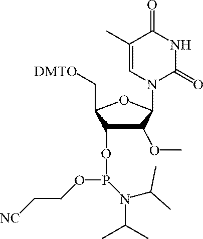 | 0.1 M in dry ACN |
| dG-Thiophosphoramidite | 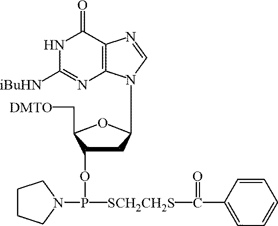 | 0.1 M in dry ACN |

### Production of Antibodies

Antibodies generated in-house are typically expressed in suspension culture of Expi293 system (ThermoFisher) according to the manufacturer’s manual. The expressed antibodies are purified via Protein A capture using MabSelectLX chromatography (GE), elution with 0.1 M citrate (pH 3.3) and dialyzed in final buffer composition of 1X PBS (Phosphate Buffered Saline, pH 7.4).

### Example 1 Transglutaminase-Mediated Conjugation

The transglutaminase-mediated conjugation was tested using different Q-tag sequences as shown in Table 3 and linkers as shown in Table 5.

### One-Step Conjugation Method Via mTG (Microbial Transglutaminase)

Q-tags were genetically linked to the C-terminus of the heavy chain of antibody (anti-CD22 or anti-CD38). To perform conjugation, the purified antibody (containing the engineered Q-tags at the C-terminus of heavy chain) were first buffer exchanged into 25 mM Tris, 150 mM NaCl pH 8. The Ab-Q-tag containing moiety and CpG were added in molar ratio of 1:1.3 and incubated overnight with a final concentration of 1% mTG (w/v) (Ajinomoto) at room temperature. Final concentration of antibody used for conjugation is generally ~ 20-25 uM. Mixture was loaded to a Q Sepharose HP (GE) equilibrated in 20% Buffer B (40 mM Tris, 2 M NaCl pH8) and 80% Buffer A (40 mM Tris, pH8). Column was washed with 5 column volumes of 20% Buffer B. Separation was achieved with using a linear gradient from 20%B to 60% B in 30 column volumes. DAR1 peak fractions (Q-tag conjugated with one CpG moiety) were pooled and concentrated followed by a gel filtration step using S200 (GE). Monomeric peak fractions were pooled and concentrated.

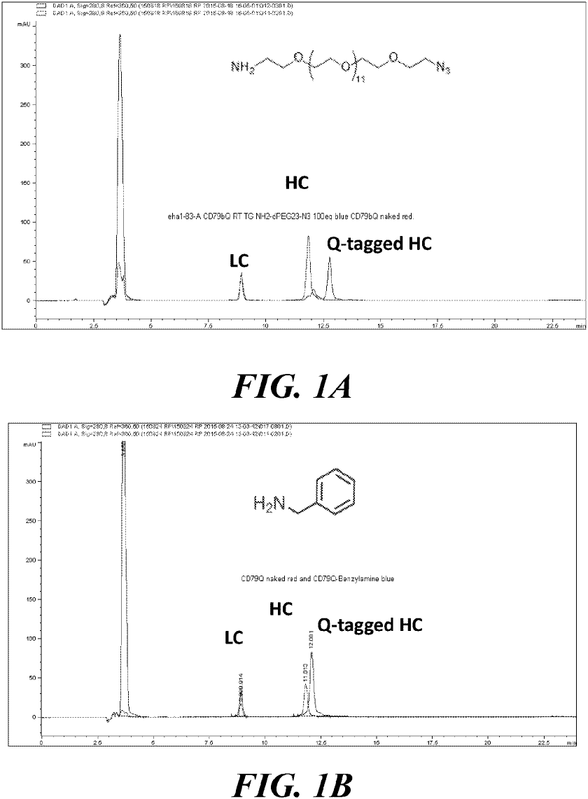

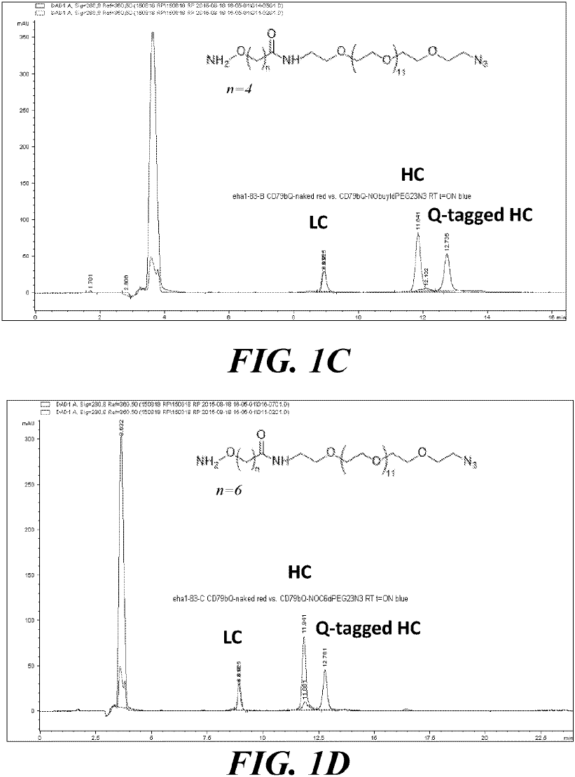

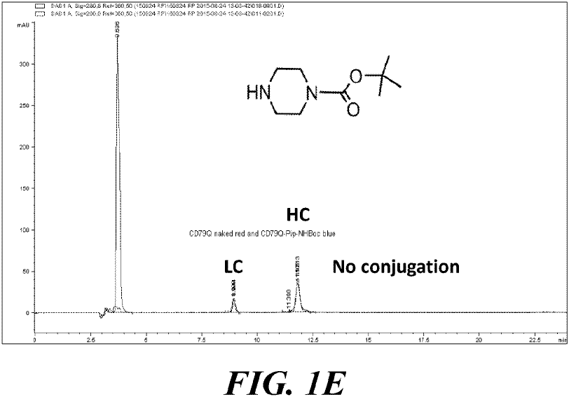

FIGS. 1A-1E show the chromatographs of the conjugation reactions with Q-tag sequence WALQRPHYSYPD and five linkers (L1-L5 as shown in Table 5Table 1). Transglutaminase-mediated reactions of the Q-tag and primary amines produced conjugates while transglutaminase-mediated reactions of the Q-tag and secondary amines did not show any detectable conjugates.

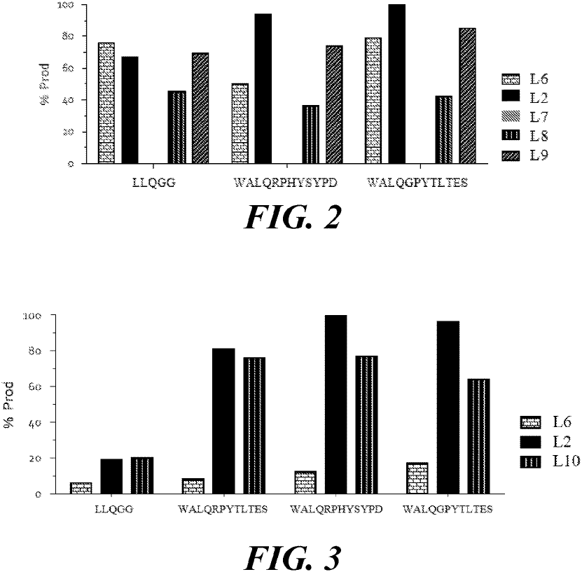

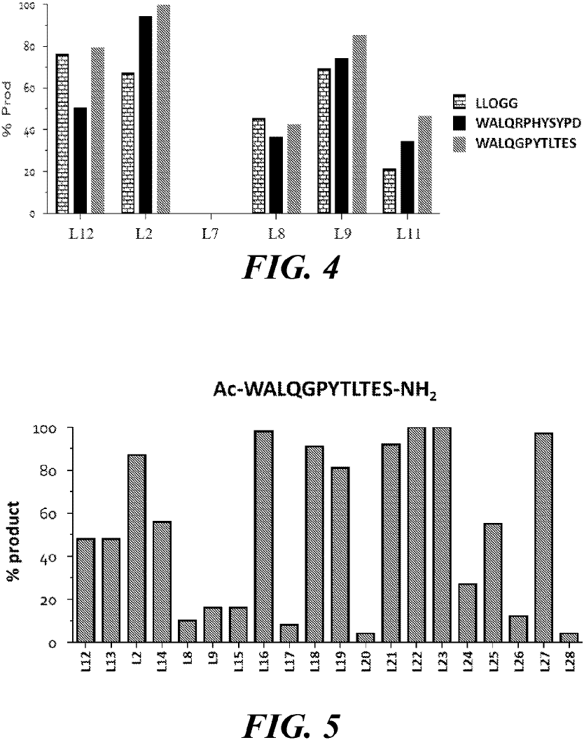

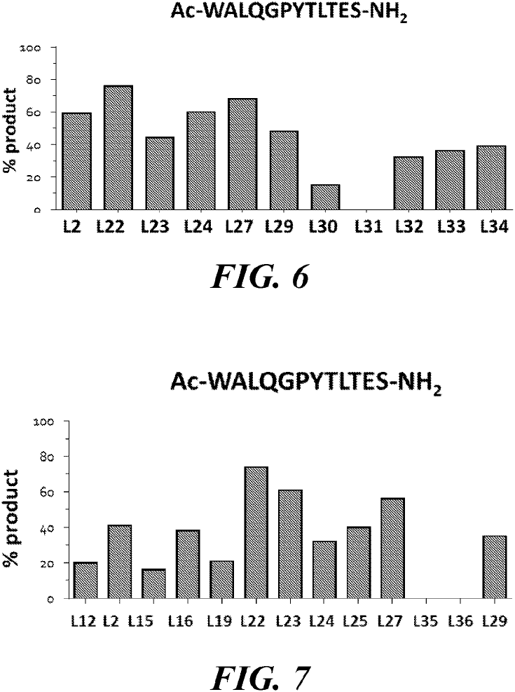

FIGS. 2-7 show the yields of the transglutaminase-mediated conjugation with various Q-tags and linkers as shown in Table 5.

TABLE 5

Linkers Used In Conjugation Reactions

| | |
|---|---|
| No. | Linker |
| L1 | 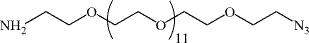 |
| L2 | 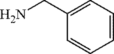 |
| L3 | 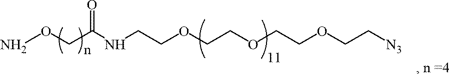 |
| L4 | 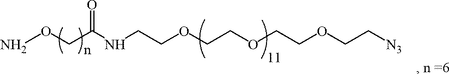 |
| L5 | 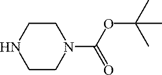 |
| L6 | 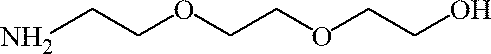 |
| L7 | 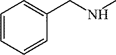 |
| L8 | 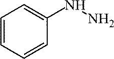 |
| L9 | 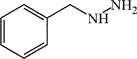 |
| L10 | 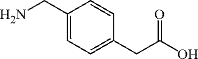 |
| L11 | 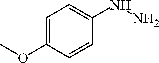 |
| L12 | 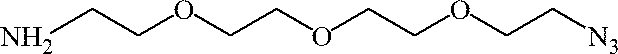 |
| L13 | 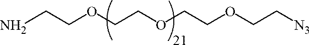 |
| L14 | 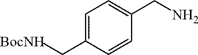 |
| L15 | 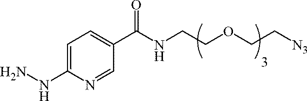 |
| L16 | 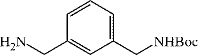 |
| L17 | 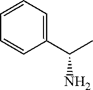 |
| L18 | 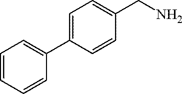 |
| L19 | 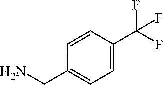 |
| L20 | 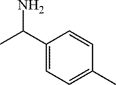 |
| L21 | 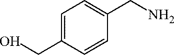 |
| L22 | 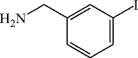 |
| L23 | 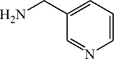 |
| L24 | 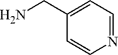 |
| L25 | 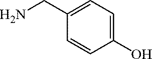 |
| L26 | 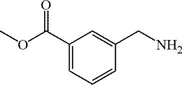 |
| L27 | 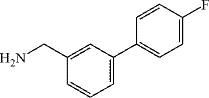 |
| L28 | 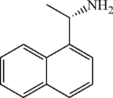 |
| L29 | 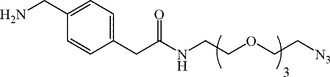 |
| L30 | 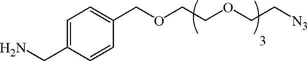 |
| L31 | 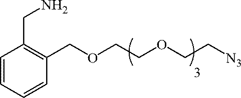 |
| L32 | 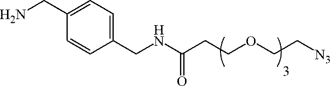 |
| L33 | 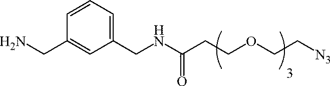 |
| L34 | 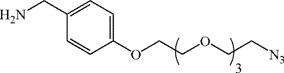 |
| L35 | 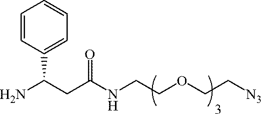 |
| L36 | 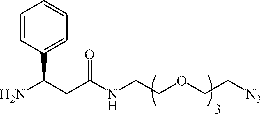 |
| L37 | 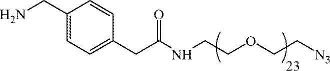 |
| L38 | 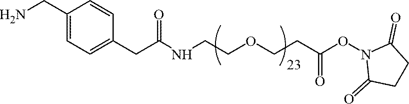 |
| L39 (L38+Y^{PTE} ) | 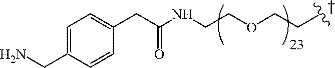 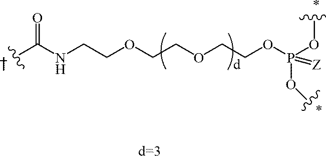 |
| L40 | 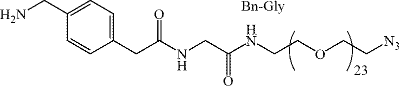 |
| L41 | 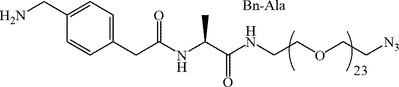 |
| L42 | 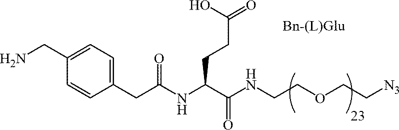 |
| L43 | 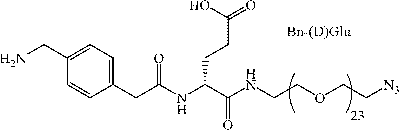 |
| L44 | 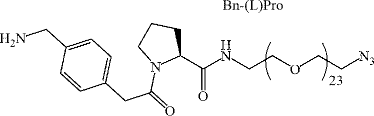 |
| L45 | 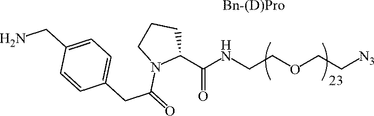 |
| L46 | 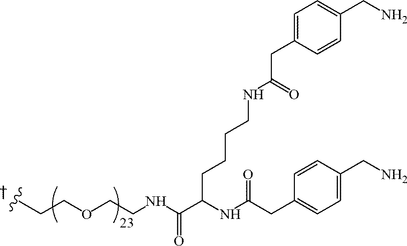 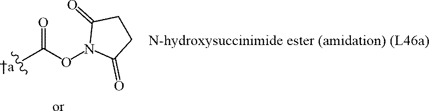 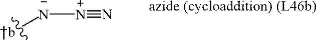 |
| L47 (L46a+ Y^{PTE}) | 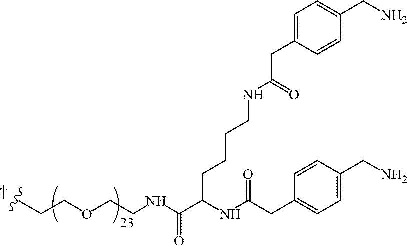 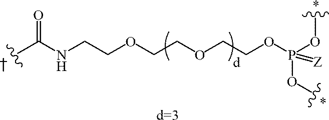 |
| L48 (L46b + Y^{PTE}) | 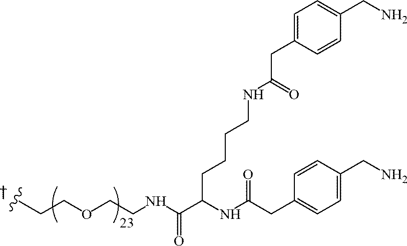 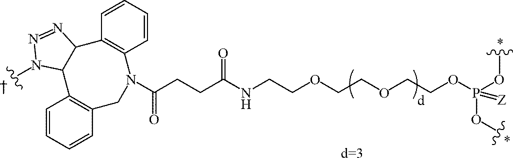 |
| L49 | 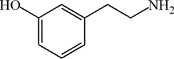 |
| L50 | 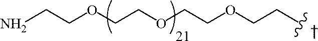 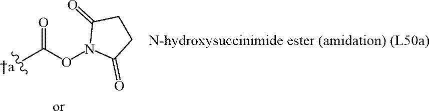 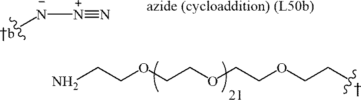 |
| L51 (L50a+Y^{P} TE) | 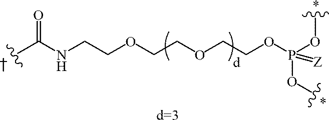 |

### Example 2 Stability of Transglutaminase-Mediated Conjugation

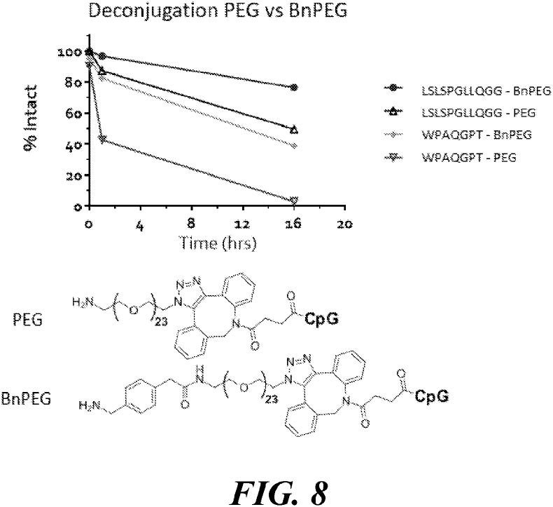

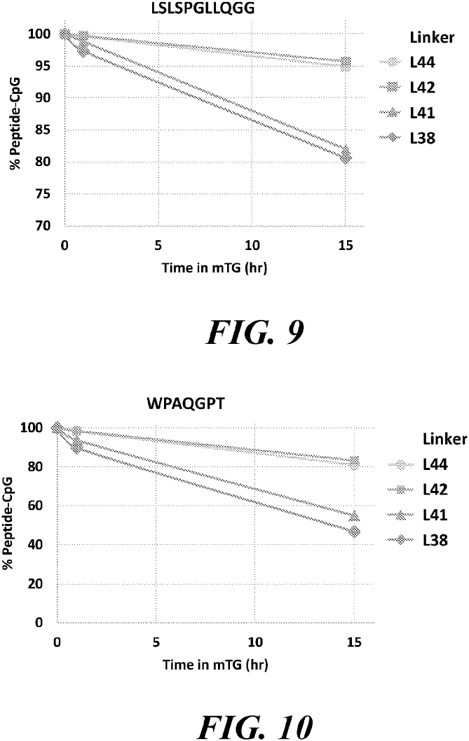

Deconjugations of the transglutaminase-mediated conjugates with different Q-tags (SEQ ID NO: 7 and 8) and linkers (L13, L37-L38, L41, L42 and L44) were monitored over 15-20 hours. Then the reaction mixture were separated using the following conditions: RP HPLC Parameters: Column: Xbridge C18, 4.6 × 150 mm, 5 µm; MPA: 50 mM TEAA in water; MPB: Acetonitrile; Gradient: 20-60% MPB in 10 min; Column temp: 60° C.; Flow: 1 mL/min; Detection: 260 nm. The observed deconjugations are shown in FIGS. 8-10.

FIGS. 8-10 shows that certain benzylamine linkers can reduce deconjugation and increase the stability of the conjugates.

### Example 3 Transglutaminase-mediated Conjugation of Multiple Q-tags on Antibody

Linkers containing one or more nucleophile sites were used to cross link an antibody containing more than one Q-tags. This can be used to modulate the numbers of payloads of each conjugate. In the following example, the design is to specifically control one payload configuration (DAR1, drug-antibody ratio of 1) (CpG, SEQ ID NO: 5). The antibody used in this example was a human anti-CD22 antibody comprising Q-tag sequence: LSLSPGLLQGG-OH (SEQ ID NO: 7) on the heavy chains. Nucleophile linkers L38, L44, L46 and L49 were used to conjugate with the antibody in the presence of transglutaminase.

The antibody was mixed with the linker in a ratio of 1:2 by weight. The transglutaminase was added at a concentration of 2 µM in PBS. The final concentration of the antibody is 25 µM. The reaction was kept in room temperature for at least 20 hours. Allocates of the reaction were quenched with 1:0.5 8 M formamide at 1, 4 and 20 hour. The reaction solution was analyzed using AEX HPLC with DNAPac PA200 column (4 × 250 mm) using solvent A (20 mM sodium phosphate pH 8, 20% ACN) and solvent B (20 mM sodium phosphate pH 8, 0.5 M NaClO₄, 20% ACN) with a gradient of 10% to 70% of solvent B in 10 minutes at 30° C.

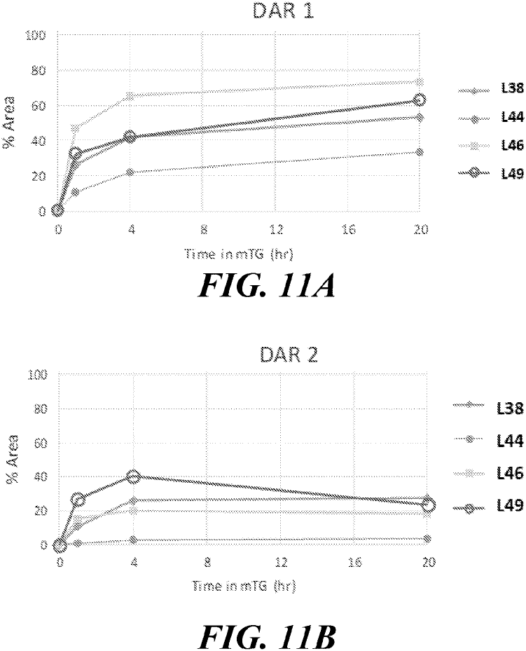

FIGS. 11A and 11B show the yields of conjugates having one linker attached to one antibody and two linkers attached to one antibody respectively. Bis-benzylamine (Y-)linker L46 cross-reacted with two Q-tags on the heavy chain preferentially to form the DAR1 conjugate, confirmed by HPLC analysis as shown in FIG. 11A. The relatively low DAR2 formation using linker L46 was shown in FIG. 11B.

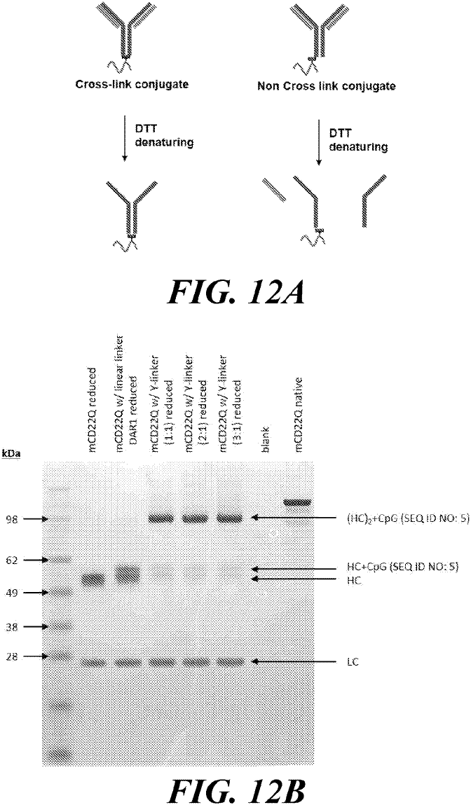

When a linker containing two nucleophile sites is used, the heavy chain of the antibody can be cross-linked. FIG. 12A shows potential antibody conjugation configurations in which the heavy chains are cross-linked or not cross-linked. In a parallel experiment to the above, mouse anti-CD22 antibody (SEQ ID NOs 19-20) was conjugated to oligonucleotide SEQ ID NO: 5, using either a linear linker (single nucleophilic site) or a branched (Y-)linker (two nucleophilic sites).

| Name | Domain | SEQ ID NO: | Sequence |
|---|---|---|---|
| mCD22Q | VH | 19 | QVQLQQPGAEIVRPGTSVKLSCKASGYTFTD YWMNWVKQRPGQGLEWFGAIDPSDSYTRY NQEFKGKATLTVDTSSTTAYMQLSSLTSEDS AVYFCARSDYTYSFYFDYWGLGTTLTVSS |
| mCD22Q | VL | 20 | DIVMTQAAFSNPVTLGTSASISCRSSKSLLHS NGITYLYWYLQKPGQSPQLLIYQMSNLASG VPDRFSSSGSGTDFTLRISRVEAEDVGVYYC AQNLELPWTFGGGTKLEIK |

Gel electrophoresis was conducted to confirm cross-linking of heavy chains and to determine the relative yield of the cross-linked conjugates and non-cross linked conjugates (FIG. 12B).

### Example 4 Transglutaminase-Mediated Conjugation

The transglutaminase-mediated conjugation was tested using different linkers and Q-tag sequences.

2 nmol of the Q-tag was added to 1 nmol of the linker in the presence of 0.04 nmol of transglutaminase in PBS. The final concentration of linker is 50 µM. Reactions were kept at room temperature and quenched with 8 M formamide at 1 hour. The reaction solution was analyzed using reverse-phase HPLC with Xbridge C18 column (4.6 × 150 mm) using solvent A (50 mM TEAA in water) and solvent B (Acetonitrile) with a gradient of 20% to 60% of solvent B in 10 minutes at 60° C. Alternatively, the reaction solution was analyzed using reverse-phase HPLC with Luna 3 µ C18 column (4.6 × 50 mm) using solvent A (0.1% TFA in water) and solvent B (0.1% TFA in Acetonitrile) with a gradient of 10% to 70% of solvent B in 10 minutes at 50° C.

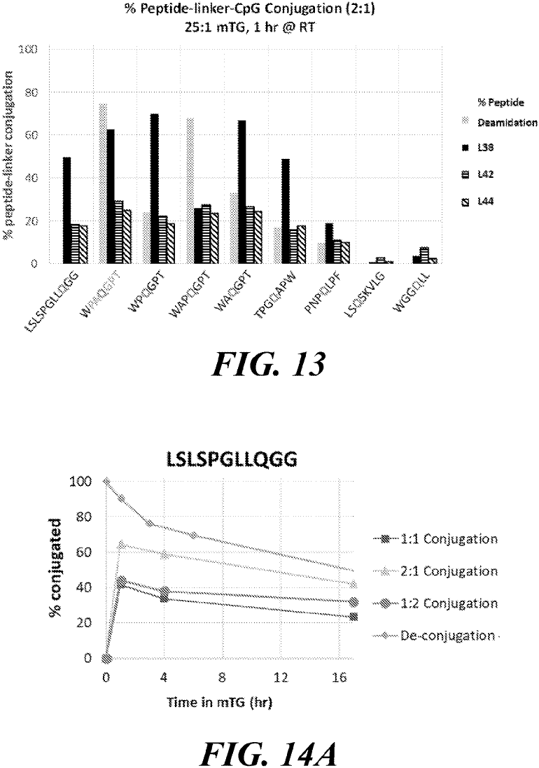

FIG. 13 shows the yields of the transglutaminase-mediated conjugation with various Q-tags (SEQ ID NO: 7-15) and linkers (L38, L42, and L44).

Further studies were carried out to evaluate the effect of variable concentration ratios of Q-tag to the linker-oligonucleotides for the transglutaminase conjugation reaction. The Q-tag LSLSPGLLQGG (SEQ ID NO: 7) and the linker-oligonucleotides were both provided to give a final concentration of either 50 µM or 100 µM, in the presence of 2 µM transglutaminase. The concentration ratios evaluated included 100 µM Q-tag to 50 µM linker-oligonucleotide (2:1 Q-tag:linker-CpG); 50 µM Q-tag to 50 µM linker-oligonucleotide (1:1 Q-tag:linker-CpG); and 50 µM Q-tag to 100 µM linker-oligonucleotide (1:2 Q-tag:linker-CpG).

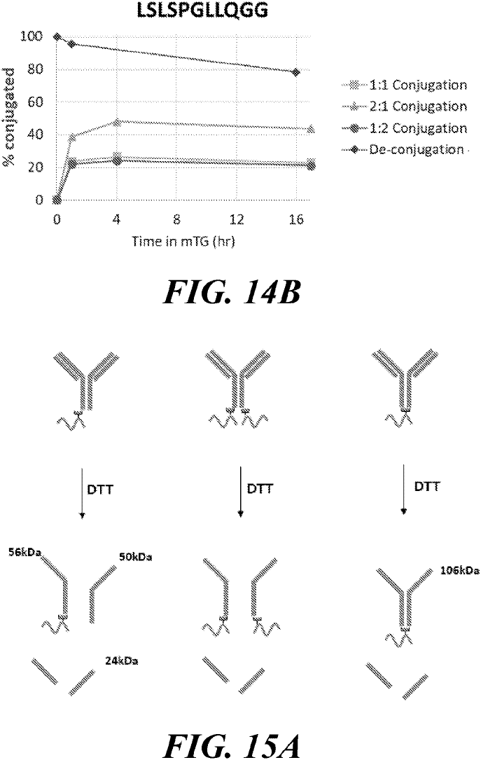

FIGS. 14A-14B show the deconjugation rates of two conjugates of linkers (L51 in FIG. 14A and L39 in FIG. 14B) with oligonucleotide sequence tsuscsgstscsgstsgsascsgstst-c3, (SEQ ID NO: 5) and Q-tag LSLSPGLLQGG (SEQ ID NO: 7) prepared at various concentration ratios of Q-tag to linker-CpG (2:1, 1:1 and 1:2).

### Example 5 Synthesis of Branched (Y-)Linker L48 and Transglutaminase-Mediated Conjugation of Multiple Q-Tags on Antibody

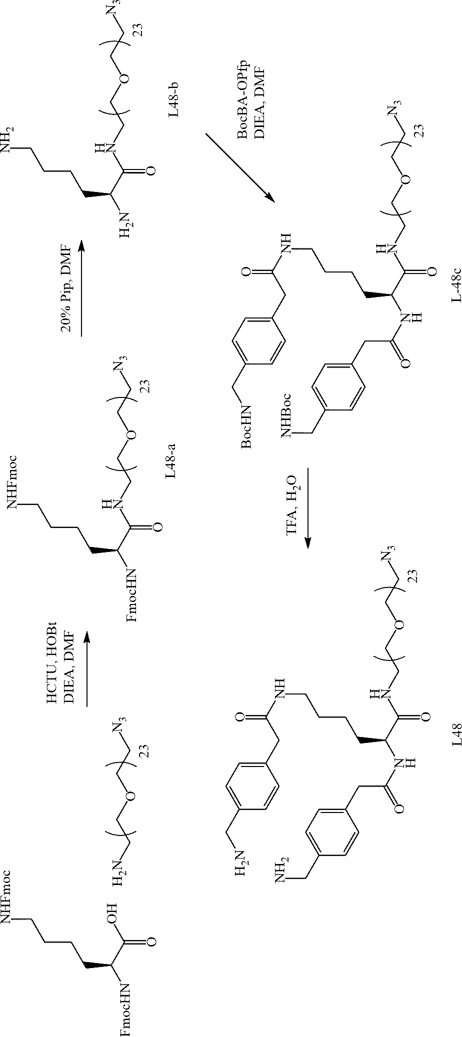

The synthetic scheme for the preparation of the branched (Y-)linker L48 is shown above. As shown in the scheme, Fmoc-protected L-lysine (400.81 mg, 678.55 µmol, 1 eq.) and H₂N-PEG₂₃-N₃ (820.54 mg, 746.40 µmol, 1.1 eq.) were mixed in DMF(5-10 mL) in the presence of DIEA (590.97 µL, 3.39 mmol, 5 eq.), HCTU (280.71 mg, 678.55 µmol, 1 eq.) and HOBt (91.69 mg, 678.55 µmol,1 eq.). The resulting PEGylated compound L48-a was isolated from the reaction mixture. The isolated intermediate L48-a was deprotected with a solution of 20% piperidine in DMF to yield the free amine L48-b. The intermediate L48-b (100 mg, 81.47 µmol, 1 eq.) was combined with Boc-protected, Pfp-activated ester (pentafluorophenyl) 2-(4-(aminomethyl)phenyl)acetic acid (77.31 mg, 179.23 µmol, 2.2 eq.) in DMF (5 mL) in the presence of DIEA (70.95 mL) to give compound L48-c. The resulting intermediate compound L-48c was deprotected with a solution of trifluoroacetic acid in water to give the final linker L48 compound. Silica gel chromatography was used for purification. TLC and mass spectrometry were used to monitor reaction progress and assess purity of the product(s) throughout.

The synthesized branched (Y-)linker L48 was evaluated for its ability to form cross-linked antibody conjugates under various experimental conditions. Stock solution of oligonucleotide sequence SEQ ID NO: 4 conjugated to branched (Y-)linker L48 were prepared at 500 µM in Tris-HCl buffer. A stock solution of anti-CD22 antibody (SEQ ID NO: 21-22) was prepared at concentration of 51.5 µM in Tris-HCl buffer.

| Name | Domain | SEQ ID NO: | Sequence |
|---|---|---|---|
| 10F4 | VH | 21 | EVQLVESGGGLVQPGGSLRLSCAASGYEFSR SWMNWVRQAPGKGLEWVGRIYPGDGDTNY SGKFKGRFTISADTSKNTAYLQMNSLRAEDT AVYYCARDGSSWDWYFDVWGQGTLVTVSS |
| 10F4 | VL | 22 | DIQMTQSPSSLSASVGDRVTITCRSSQSIVHS VGNTFLEWYQQKPGKAPKLLIYKVSNRFSG VPSRFSGSGSGTDFTLTISSLQPEDFATYYCF QGSQFPYTFGQGTKVEIK |

The stock solutions of the oligonucleotide-linker and antibody were combined at various volumes to give ratios of 1:1, 1:5 and 1:10 of the antibody to the oligonucleotide-linker (Ab:Y-CpG) by molarity. The transglutaminase was added to each mixture at a concentration of 2 µM in Tris-HCl to give a molar ratio of antibody-to-transglutaminase of 50:1. The reaction mixtures were stirred at 22° C. overnight. After stirring, the mixtures were analyzed by gel electrophoresis with (reduced) and without (non-reduced) prior dithiothreitol (DTT) treatment.

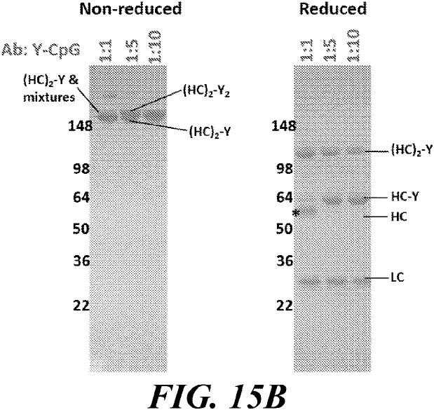

FIG. 15A shows the various possible configurations of antibody-linker-oligonucleotide conjugation products when linker L48 containing two nucleophile sites was used (FIG. 15A). Gel electrophoresis was conducted to confirm L48 cross-linking of heavy chains (HC) and to determine the relative yield of the cross-linked conjugates and non-cross linked conjugates (FIG. 15B).

In FIG. 15B, the 1:1 (Ab:L48-CpG) molar ratio yielded higher levels of cross-linked heavy chains with DAR1 [(HC)₂—Y DAR1] than the other two molar ratios evaluated. Reaction mixtures using molar ratios of 1:5 (Ab:L48-CpG) and 1:10 (Ab:L48-CpG) both gave mixtures of cross-linked heavy chains with DAR1 [(HC)₂—Y DAR1] and another conjugation product, (HC)₂—Y₂ DAR2.

1. A conjugate of Formula (A1): 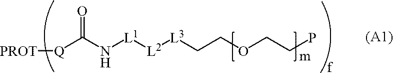
or a stereoisomer, a mixture of two or more diastereomers, a tautomer, or a mixture of two or more tautomers thereof; or a pharmaceutically acceptable salt, solvate, or hydrate thereof; wherein:
Q is a glutamine residue, wherein each glutamine residue independently is part of the sequence of PROT or is part of a Q-tag peptide sequence attached to PROT;
PROT is a protein connected to the rest of the conjugate via one or more glutamine residues Q;
each L¹ is independently unsubstituted or substituted alkyl,
each L² is unsubstituted or substituted aryl, or unsubstituted or substituted heteroaryl,
each L³ is independently absent or a linker moiety,
f is an integer selected from the group consisting of 1-20,
m is an integer selected from the group consisting of 0-50, and
P is an immunomodulating oligonucleotide.

2. The conjugate of claim 1, wherein the protein is an antibody.

3-9. (canceled)

10. The conjugate of claim 1, wherein f is 1 or 2.

11. The conjugate of claim 1, wherein P is 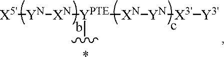
wherein
b and c are each independently an integer from 1 to 25; with the proviso that the sum of b and c is at least 5; 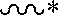 [SMILES: *C]
indicates the point of attachment of the immunomodulating oligonucleotide P to the rest of the conjugate;
X^{5′} is a 5′ terminal nucleoside having the structure 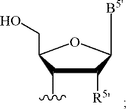
X^{3′} is a 3′ terminal nucleoside having the structure 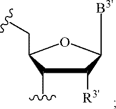
Y^{PTE} is an internucleoside phosphotriester having the structure 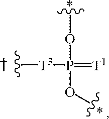
wherein * indicates the points of attachment to the rest of the oligonucleotide and † indicates the point of attachment to the rest of the conjugate;
Y^{3′} is a terminal phosphotriester having the structure 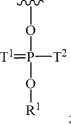
each X^{N} is independently a nucleoside having the structure 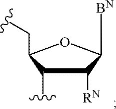
each Y^{N} is independently an internucleoside linker having the structure 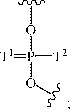
wherein each B
^{N} is independently a modified or unmodified nucleobase;
each R^{N} is independently —H or —O—C₁₋₄-alkyl, wherein the C₁₋₄-alkyl of the —O—C₁₋₄-alkyl is further optionally substituted by —O—C₁-C₄-alkyl;
B^{5′} and B³’ are independently a modified or unmodified nucleobase;
R⁵’ and R³’ are independently —H or —O—C₁-C₄-alkyl, wherein the C₁₋₄-alkyl of the —O—C₁₋₄-alkyl is further optionally substituted by —O—C₁-C₄-alkyl;
each T₁ is independently O or S;
each T₂ is independently O⁻ or S⁻; and
T₃ is a group comprising an oligoethylene glycol moiety; and
R¹ is C₁₋₄-alkylene-hydroxy.

12. The conjugate of claim 11, wherein (i) P comprises at least one modified nucleoside X^{N}; (ii) P has at least one modified internucleoside linker Y^{N}, wherein at least one of T¹ or T² is S; or (iii) both (i) and (ii).

13-14. (canceled)

15. The conjugate of claim 11, wherein P comprises one or more CpG sites.

16-19. (canceled)

20. The conjugate of claim 11, wherein Y^{PTE} is: 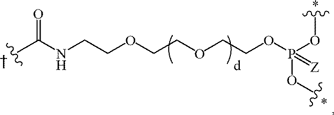 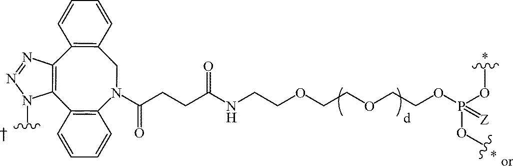 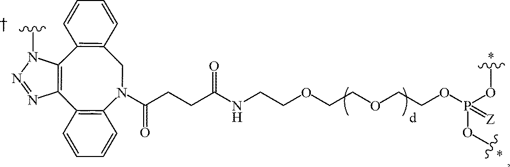
wherein Z is O or S; d is an integer from 0 to 95; the two 〰 * on the right side of the structure indicate the points of attachment to the adjacent nucleosides X
^{N} in the oligonucleotide P, and the one † on the left side of the structure indicates the point of attachment to the rest of the conjugate.

21-37. (canceled)

38. A conjugate of Formula (B1): 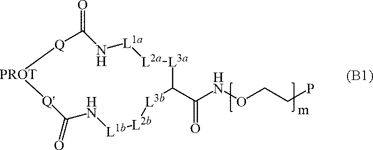
or a stereoisomer, a mixture of two or more diastereomers, a tautomer, or a mixture of two or more tautomers thereof; or a pharmaceutically acceptable salt, solvate, or hydrate thereof; wherein:
Q and Q′ are each a glutamine residue, wherein each glutamine residue independently is part of the sequence of PROT or is part of a Q-tag peptide sequence attached to PROT;
PROT is a protein connected to the rest of the conjugate via Q and Q′;
L^{1a} and L^{1b} are independently unsubstituted or substituted alkyl,
L^{2a} and L^{2b} are independently absent, unsubstituted or substituted alkyl, unsubstituted or substituted aryl, or unsubstituted or substituted heteroaryl;
L^{3a} and L^{3b} are independently absent or a linker moiety,
m is an integer selected from the group consisting of 0-50, and
P is an immunomodulating oligonucleotide.

39. The conjugate of claim 38, wherein the Q-tag peptide sequences comprising Q and Q′ comprise the same peptide sequence.

40. The conjugate of claim 38, wherein the protein is an antibody.

41-47. (canceled)

48. The conjugate of claim 38, wherein P is 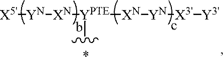
wherein
b and c are each independently an integer from 1 to 25; with the proviso that the sum of b and c is at least 5; 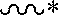 [SMILES: *C]
indicates the point of attachment of the immunomodulating oligonucleotide P to the rest of the conjugate;
X⁵′ is a 5′ terminal nucleoside having the structure 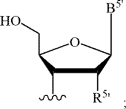
X³′ is a 3′ terminal nucleoside having the structure 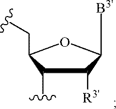
Y^{PTE} is an internucleoside phosphotriester having the structure 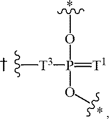
wherein * indicates the points of attachment to the rest of the oligonucleotide and † indicates the point of attachment to the rest of the conjugate;
Y^{3′} is a terminal phosphotriester having the structure 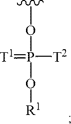
each X^{N} is independently a nucleoside having the structure 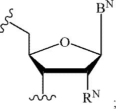
each Y^{N} is independently an internucleoside linker having the structure 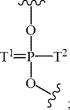
wherein each B
^{N} is independently a modified or unmodified nucleobase;
each R^{N} is independently —H or —O—C₁₋₄-alkyl, wherein the C₁₋₄-alkyl of the —O—C₁₋₄-alkyl is further optionally substituted by —O—C₁-C₄-alkyl;
B⁵′ and B³′ are independently a modified or unmodified nucleobase;
R⁵′ and R³′ are independently —H or —O—C₁-C₄-alkyl, wherein the C₁₋₄-alkyl of the —O—C₁₋₄-alkyl is further optionally substituted by —O—C₁-C₄-alkyl;
each T₁ is independently O or S;
each T₂ is independently O⁻ or S⁻; and
T₃ is a group comprising an oligoethylene glycol moiety; and
R¹ is C₁₋₄-alkylene-hydroxy.

49. The conjugate of claim 48, wherein (i) P comprises at least one modified nucleoside X^{N}; (ii) P has at least one modified internucleoside linker Y^{N}, wherein at least one of T¹ or T² is S; or (iii) both (i) and (ii).

50-51. (canceled)

52. The conjugate of claim 48, wherein P comprises one or more CpG sites.

53-56. (canceled)

57. The conjugate of claim 48, wherein Y^{PTE} is: 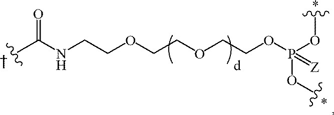 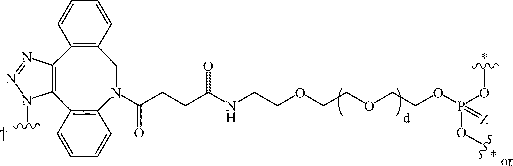 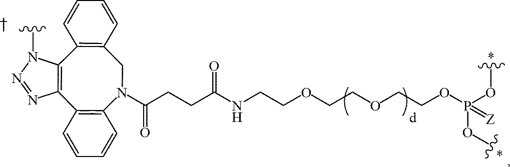
wherein Z is O or S; d is an integer from 0 to 95; the two 〰 * on the right side of the structure indicate the points of attachment to the adjacent nucleosides X
^{N} in the oligonucleotide P, and the one † on the left side of the structure indicates the point of attachment to the rest of the conjugate.

58-60. (canceled)

61. The conjugate of claim 38, wherein one or both of L^{1a} and L^{1b} are substituted by an unsubstituted or substituted aryl.

62. (canceled)

63. The conjugate of claim 38, wherein one or both of L^{2a} and L^{2b} are absent.

64. The conjugate of claim 38, wherein one or both of L^{2a} and L^{2b} are independently unsubstituted or substituted aryl or unsubstituted or substituted heteroaryl.

65. The conjugate of claim 38, wherein one or both of L^{3a} and L^{3b} are linker moieties.

66-73. (canceled)

74. A method of preparing of a conjugate of Formula (A1) according to claim 1: 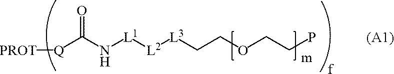
or a stereoisomer, a mixture of two or more diastereomers, a tautomer, or a mixture of two or more tautomers thereof; or a pharmaceutically acceptable salt, solvate, or hydrate thereof;
comprising reacting (1) a compound of Formula (I): 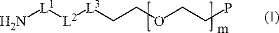
or a stereoisomer, a mixture of two or more diastereomers, a tautomer, or a mixture of two or more tautomers thereof; or a pharmaceutically acceptable salt, solvate, or hydrate thereof,
and (2) a protein comprising one or more glutamine residues in the presence of a transglutaminase, wherein:
Q is a glutamine residue, wherein each glutamine residue independently is part of the sequence of PROT or is part of a Q-tag peptide sequence attached to PROT;
PROT is a protein connected to the rest of the conjugate via one or more glutamine residues Q;
each L¹ is independently unsubstituted or substituted alkyl,
each L² is unsubstituted or substituted aryl, or unsubstituted or substituted heteroaryl,
each L³ is independently absent or a linker moiety,
f is an integer selected from the group consisting of 1-20,
m is an integer selected from the group consisting of 0-50, and
P is an immunomodulating oligonucleotide.

75. A method of preparing a conjugate of Formula (B1) according to claim 38: 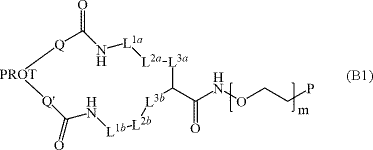
or a stereoisomer, a mixture of two or more diastereomers, a tautomer, or a mixture of two or more tautomers thereof; or a pharmaceutically acceptable salt, solvate, or hydrate thereof;
comprising reacting (1) a compound of Formula (II): 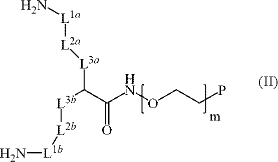
or a stereoisomer, a mixture of two or more diastereomers, a tautomer, or a mixture of two or more tautomers thereof; or a pharmaceutically acceptable salt, solvate, or hydrate thereof; and (2) a protein comprising two or more glutamine residues in the presence of a transglutaminase, wherein:
Q and Q′ are each a glutamine residue, wherein each glutamine residue independently is part of the sequence of PROT or is part of a Q-tag peptide sequence attached to PROT;
PROT is a protein connected to the rest of the conjugate via Q and Q′;
L^{1a} and L^{1b} are independently unsubstituted or substituted alkyl,
L^{2a} and L^{2b} are independently absent, unsubstituted or substituted alkyl, unsubstituted or substituted aryl or unsubstituted or substituted heteroaryl,
L^{3a} and L^{3b} are independently absent or a linker moiety,
m is an integer selected from the group consisting of 0-50, and
P is an immunomodulating oligonucleotide.

76-81. (canceled)

82. A compound of Formula (III): 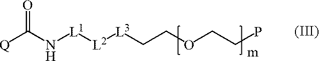
or a stereoisomer, a mixture of two or more diastereomers, a tautomer, or a mixture of two or more tautomers thereof; or a pharmaceutically acceptable salt, solvate, or hydrate thereof; wherein:
Q is a glutamine residue, wherein the glutamine residue is part of a Q-tag peptide sequence;
L¹ is unsubstituted or substituted alkyl,
L² unsubstituted or substituted aryl or unsubstituted or substituted heteroaryl,
L³ is absent or a linker moiety,
m is an integer selected from the group consisting of 0-50, and
P is an immunomodulating oligonucleotide.
